SEQUENCE LISTING (39 sequences)

SEQ ID NO: 1 - protein, 213 aa, synthetic construct
  REGION 1..213: note = source = /note="Description of Artificial Sequence: Syntheticpolypeptide"
SSALTQPSALSVTKGQTAKITCQGDLLPRHYAHWYQQKTGQAPKLIVYDDDIRPSGIPERFSGSNSGGVATLTIAGAQAEDEADYHCQSADSNDDAVFGGGTHLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 2 - protein, 455 aa, synthetic construct
  REGION 1..455: note = source = /note="Description of Artificial Sequence: Syntheticpolypeptide"
EVQVQESGPGLVKPSQTLSLTCTVSGGSITTSYYAWSWIRQPPGKGLEWMGDIANDGSTYYSPSLESRTSISRDTSKNQFSLQLSSVTPEDTAVYYCARLRSLYTDYDPHYYDYWGQGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 3 - protein, 213 aa, synthetic construct
  REGION 1..213: note = source = /note="Description of Artificial Sequence: Syntheticpolypeptide"
SYELTQPPALSVSPGQTARITCQGDLLPRHYAHWYQQKTGQAPKLVIYDDDIRPSGIPERFSGSSSGTMATLTISGAQAEDEADYHCQSADSNDDAVFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 4 - protein, 454 aa, synthetic construct
  REGION 1..454: note = source = /note="Description of Artificial Sequence: Syntheticpolypeptide"
QVQLQESGPGLVKPSQTLSLTCTVSGGSISTSYYAWSWIRQPPGKGLEWIGDIANEGSTYYSPSLESRVTISRDTSKNQFSLQLSSVTAADTAVYYCARLRSLYTDYDPHYYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 5 - protein, 125 aa, synthetic construct
  REGION 1..125: note = source = /note="Description of Artificial Sequence: Syntheticpolypeptide"
QVQLQESGPGLVKPSQTLSLTCTVSGGSISTSYYAWSWIRQPPGKGLEWIGDIANEGSTYYSPSLKSRVTISRDTSKNQFSLQLSSVTAADTAVYYCARLRSLYTDYDPHYYDYWGQGTLVTVSS

SEQ ID NO: 6 - protein, 107 aa, synthetic construct
  REGION 1..107: note = source = /note="Description of Artificial Sequence: Syntheticpolypeptide"
SYELTQPPALSVSPGQTARITCQGNLLPRHYAHWYQQKTGQAPKLVIYDDNIRPSGIPERFSGSSSGTTATLTISGAQAEDEADYHCQSASSNDDAVFGGGTQLTVL

SEQ ID NO: 7 - skipped

SEQ ID NO: 8 - protein, 107 aa, synthetic construct
  REGION 1..107: note = source = /note="Description of Artificial Sequence: Syntheticpolypeptide"
SYELTQPPALSVSPGQTARITCQGNLLPRHYAHWYQQKTGQAPKLVIYDDNIRPSGIPERFSGSSSGTTATLTISGAQAEDEADYHCQSADLNDDAVFGGGTQLTVL

SEQ ID NO: 9 - protein, 11 aa, synthetic construct
  REGION 1..11: note = source = /note="Description of Artificial Sequence: Syntheticpeptide"
QGDLLPRHYAH

SEQ ID NO: 10 - protein, 7 aa, synthetic construct
  REGION 1..7: note = source = /note="Description of Artificial Sequence: Syntheticpeptide"
DDDIRPS

SEQ ID NO: 11 - protein, 10 aa, synthetic construct
  REGION 1..10: note = source = /note="Description of Artificial Sequence: Syntheticpeptide"
QSADSNDDAV

SEQ ID NO: 12 - protein, 5 aa, synthetic construct
  REGION 1..5: note = source = /note="Description of Artificial Sequence: Syntheticpeptide"
YYAWS

SEQ ID NO: 13 - protein, 16 aa, synthetic construct
  REGION 1..16: note = source = /note="Description of Artificial Sequence: Syntheticpeptide"
DIANDGSTYYSPSLES

SEQ ID NO: 14 - protein, 15 aa, synthetic construct
  REGION 1..15: note = source = /note="Description of Artificial Sequence: Syntheticpeptide"
LRSLYTDYDPHYYDY

SEQ ID NO: 15 - protein, 16 aa, synthetic construct
  REGION 1..16: note = source = /note="Description of Artificial Sequence: Syntheticpeptide"
DIANEGSTYYSPSLKS

SEQ ID NO: 16 - protein, 11 aa, synthetic construct
  REGION 1..11: note = source = /note="Description of Artificial Sequence: Syntheticpeptide"
QGNLLPRHYAH

SEQ ID NO: 17 - protein, 7 aa, synthetic construct
  REGION 1..7: note = source = /note="Description of Artificial Sequence: Syntheticpeptide"
DDNIRPS

SEQ ID NO: 18 - protein, 10 aa, synthetic construct
  REGION 1..10: note = source = /note="Description of Artificial Sequence: Syntheticpeptide"
QSASSNDDAV

SEQ ID NO: 19 - protein, 10 aa, synthetic construct
  REGION 1..10: note = source = /note="Description of Artificial Sequence: Syntheticpeptide"
QSADLNDDAV

SEQ ID NO: 20 - protein, 16 aa, synthetic construct
  REGION 1..16: note = source = /note="Description of Artificial Sequence: Syntheticpeptide"
DIANEGSTYYSPSLES

SEQ ID NO: 21 - protein, 19 aa, synthetic construct
  REGION 1..19: note = source = /note="Description of Artificial Sequence: Syntheticpeptide"
MGWSCIILFLVATATGVHS

SEQ ID NO: 22-25 - skipped (4 entries)

SEQ ID NO: 26 - protein, 123 aa, synthetic construct
  REGION 1..123: note = source = /note="Description of Artificial Sequence: Syntheticpolypeptide"
SSALTQPSALSVTKGQTAKITCQGDLLPRHYAHWYQQKTGQAPKLIVYDDDIRPSGIPERFSGSNSGGVATLTIAGAQAEDEADYHCQSADSNDDAVFGGGTHLTVLSQPKAAPSVTLFPPSS

SEQ ID NO: 27 - protein, 125 aa, synthetic construct
  REGION 1..125: note = source = /note="Description of Artificial Sequence: Syntheticpolypeptide"
EVQVQESGPGLVKPSQTLSLTCTVSGGSITTSYYAWSWIRQPPGKGLEWMGDIANDGSTYYSPSLESRTSISRDTSKNQFSLQLSSVTPEDTAVYYCARLRSLYTDYDPHYYDYWGQGTQVTVSS

SEQ ID NO: 28 - protein, 118 aa, synthetic construct
  REGION 1..118: note = source = /note="Description of Artificial Sequence: Syntheticpolypeptide"
SYELTQPPALSVSPGQTARITCQGDLLPRHYAHWYQQKTGQAPKLVIYDDDIRPSGIPERFSGSSSGTMATLTISGAQAEDEADYHCQSADSNDDAVFGGGTQLTVLGQPKAAPSVTL

SEQ ID NO: 29 - protein, 125 aa, synthetic construct
  REGION 1..125: note = source = /note="Description of Artificial Sequence: Syntheticpolypeptide"
QVQLQESGPGLVKPSQTLSLTCTVSGGSISTSYYAWSWIRQPPGKGLEWIGDIANEGSTYYSPSLESRVTISRDTSKNQFSLQLSSVTAADTAVYYCARLRSLYTDYDPHYYDYWGQGTLVTVSS

SEQ ID NO: 30 - protein, 213 aa, synthetic construct
  REGION 1..213: note = source = /note="Description of Artificial Sequence: Syntheticpolypeptide"
SSALTQPPALSVSKGQTARITCQGNLLPRHYAHWYQQKTGQAPKLIVYDDNIRPSGIPERFSGSNSGGVATLTISGAQAEDEADYHCQSADSNDDAVFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 31 - protein, 454 aa, synthetic construct
  REGION 1..454: note = source = /note="Description of Artificial Sequence: Syntheticpolypeptide"
QVQVQESGPGLVKPSQTLSLTCTVSGGSISTSYYAWSWIRQPPGKGLEWMGDIANDGSTYYSPSLESRVTISRDTSKNQFSLQLSSVTAQDTAVYYCARLRSLYTDYDPHYYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 32 - protein, 213 aa, synthetic construct
  REGION 1..213: note = source = /note="Description of Artificial Sequence: Syntheticpolypeptide"
SSALTQPSALSVSKGQTARITCQGNLLPRHYAHWYQQKTGQAPKLIVYDDNIRPSGIPERFSGSNSGGVATLTISGAQAEDEADYHCQSADSNDDAVFGGGTHLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 33 - protein, 454 aa, synthetic construct
  REGION 1..454: note = source = /note="Description of Artificial Sequence: Syntheticpolypeptide"
QVQVQESGPGLVKPSQTLSLTCTVSGGSISTSYYAWSWIRQPPGKGLEWMGDIANDGSTYYSPSLESRVTISRDTSKNQFSLQLSSVTAQDTAVYYCARLRSLYTDYDPHYYDYWGQGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 34 - protein, 125 aa, synthetic construct
  REGION 1..125: note = source = /note="Description of Artificial Sequence: Syntheticpolypeptide"
QVQVQESGPGLVKPSQTLSLTCTVSGGSISTSYYAWSWIRQPPGKGLEWMGDIANDGSTYYSPSLESRVTISRDTSKNQFSLQLSSVTAQDTAVYYCARLRSLYTDYDPHYYDYWGQGTLVTVSS

SEQ ID NO: 35 - protein, 107 aa, synthetic construct
  REGION 1..107: note = source = /note="Description of Artificial Sequence: Syntheticpolypeptide"
SSALTQPPALSVSKGQTARITCQGNLLPRHYAHWYQQKTGQAPKLIVYDDNIRPSGIPERFSGSNSGGVATLTISGAQAEDEADYHCQSADSNDDAVFGGGTQLTVL

SEQ ID NO: 36 - protein, 125 aa, synthetic construct
  REGION 1..125: note = source = /note="Description of Artificial Sequence: Syntheticpolypeptide"
QVQVQESGPGLVKPSQTLSLTCTVSGGSISTSYYAWSWIRQPPGKGLEWMGDIANDGSTYYSPSLESRVTISRDTSKNQFSLQLSSVTAQDTAVYYCARLRSLYTDYDPHYYDYWGQGTQVTVSS

SEQ ID NO: 37 - protein, 107 aa, synthetic construct
  REGION 1..107: note = source = /note="Description of Artificial Sequence: Syntheticpolypeptide"
SSALTQPSALSVSKGQTARITCQGNLLPRHYAHWYQQKTGQAPKLIVYDDNIRPSGIPERFSGSNSGGVATLTISGAQAEDEADYHCQSADSNDDAVFGGGTHLTVL

SEQ ID NO: 38 - protein, 19 aa, synthetic construct
  REGION 1..19: note = source = /note="Description of Artificial Sequence: Syntheticpeptide"
MAWTPLLLPLLTFCTVSEA

SEQ ID NO: 39 - protein, 19 aa, synthetic construct
  REGION 1..19: note = source = /note="Description of Artificial Sequence: Syntheticpeptide"
MKHLWFFLLLVAAPRWVLS

(19) United States
(12) Patent Application Publication
(10) Pub. No.: US 2024/0034779 A1
(43) Pub. Date: Feb. 1, 2024

(54) ANTIBODIES TARGETING COMPLEMENT FACTOR D AND USES THEROF

(71) Applicant: Takeda Pharmaceutical Company Limited, Osaka (JP)

(72) Inventors: Sebastien Vallee, Lexington, MA (US); Madhusudan Natarajan, Lexington, MA (US); Rizwana Islam, Lexington, MA (US); Muthuraman Meiyappan, Lexington, MA (US); Angela Norton, Lexington, MA (US); Niksa Kastrapeli, Lexington, MA (US); Christophe Blanchetot, Destelbergen (BE); Christophe Steyaert, Wondelgem (BE)

(21) Appl. No.: 18/482,522

(22) Filed: Oct. 6, 2023

Related U.S. Application Data
(63) Continuation of application No. PCT/IB2022/053334, filed on Apr. 8, 2022.
(60) Provisional application No. 63/173,092, filed on Apr. 9, 2021.

(51) Int. Cl. C07K 16/18; C12N 15/63; A61P 7/00

(52) U.S. Cl. CPC C07K 16/18; C12N 15/63; A61P 7/00; C07K 2317/565; C07K 2317/52; C07K 2317/92; C07K 2317/14

(57) ABSTRACT

Antibodies that specifically bind to complement factor D in a pH sensitive manner, are described, as well as methods of making and using such antibodies.

### CROSS-REFERENCE TO RELATED APPLICATIONS

This application claims priority to U.S. Provisional Application No. 63/173,092, filed Apr. 9, 2021, the contents of which is hereby incorporated by reference in its entirety.

### INCORPORATION-BY-REFERENCE OF SEQUENCE LISTING

The present application is filed along with a Sequence Listing in electronic format. The Sequence Listing is provided as an ASCII text file entitled SHR-2005WO_SL.txt, created Apr. 7, 2022, which is 41,684 bytes in size. The information in the electronic format of the Sequence Listing is incorporated by reference in its entirety.

### BACKGROUND

The complement system includes a number of proteins able to interact with pathogens and facilitate pathogen clearance. This system plays important roles in various biological processes such as inflammation, and dysfunction of complement can result in or contribute to disease. Various medical conditions resulting from aberrant complement activity could be treated by administration of an antibody or other binding molecule capable of binding complement factors.

### SUMMARY OF INVENTION

There is a need in the art for developing therapeutic molecules to treat patients having complement-related conditions. The present invention addresses this need by providing therapeutics that target complement factor D (CFD). In one aspect, the present invention provides an isolated antibody that specifically binds to complement factor D (CFD). In some embodiments, the isolated antibody that specifically binds to complement factor D (CFD) comprises a heavy chain variable region amino acid sequence having at least 90%, 95%. 98%, or 99% sequence identity to SEQ ID NO: 5, 27, 29, 34 or 36; and/or a light chain variable region amino acid sequence having at least 90%, 95%, 98%, or 99% sequence identity to SEQ ID NO: 6, 8, 26, 28, 35 or 37.

In some embodiments, the isolated antibody that specifically binds to complement factor D (CFD) comprises a heavy chain variable region amino acid sequence that is identical to SEQ ID NO: 5, 27, 29, 34 or 36; and/or a light chain variable region amino acid sequence that is identical to SEQ ID NO: 6, 8, 26, 28, 35 or 37.

In some embodiments, the isolated antibody comprises a heavy chain CDR1 comprising the amino acid sequence YYAWS (SEQ ID NO: 12); heavy chain CDR2 comprising the amino acid sequence DIANEGSTYYSPSLKS (SEQ ID NO: 15); and/or heavy chain CDR3 comprising the amino acid sequence LRSLYTDYDPHYYDY (SEQ ID NO: 14).

In some embodiments, the isolated antibody comprises a heavy chain CDR1 comprising the amino acid sequence YYAWS (SEQ ID NO: 12); heavy chain CDR2 comprising the amino acid sequence DIANEGSTYYSPSLES (SEQ ID NO: 20); and/or heavy chain CDR3 comprising the amino acid sequence LRSLYTDYDPHYYDY (SEQ ID NO: 14).

In some embodiments, the isolated antibody comprises a heavy chain CDR1 comprising the amino acid sequence YYAWS (SEQ ID NO: 12); a heavy chain CDR2 comprising the amino acid sequence DIANDGSTYYSPSLES (SEQ ID NO: 13); and/or a heavy chain CDR3 comprising the amino acid sequence LRSLYTDYDPHYYDY (SEQ ID NO: 14).

In some embodiments, the isolated antibody comprises a light chain variable region amino acid sequence having at least 85%, 90%, 95%, 98%, or 99% sequence identity to SEQ ID NO: 6, 8, 26, 28, 35 or 37.

In some embodiments, the isolated antibody comprises a light chain variable region amino acid sequence that is identical to SEQ ID NO: 6, 8, 26, 28, 35 or 37.

In some embodiments, the isolated antibody comprises a light chain CDR1 comprising the amino acid sequence QGNLLPRHYAH (SEQ ID NO: 16); a light chain CDR2 comprising the amino acid sequence DDNIRPS (SEQ ID NO: 17); and/or light chain CDR3 comprising the amino acid sequence QSASSNDDAV (SEQ ID NO: 18).

In some embodiments, the isolated antibody comprises a heavy chain CDR1 comprising the amino acid sequence YYAWS (SEQ ID NO: 12); a heavy chain CDR2 comprising the amino acid sequence DIANEGSTYYSPSLKS (SEQ ID NO: 15); a heavy chain CDR3 comprising the amino acid sequence LRSLYTDYDPHYYDY (SEQ ID NO: 14); a light chain CDR1 comprising the amino acid sequence QGNLLPRHYAH (SEQ ID NO: 16); a light chain CDR2 comprising the amino acid sequence DDNIRPS (SEQ ID NO: 17); and/or a light chain CDR3 comprising the amino acid sequence QSASSNDDAV (SEQ ID NO: 18).

In some embodiments, the isolated antibody comprises a heavy chain variable region amino acid sequence that is identical to SEQ ID NO: 5; and a light chain variable region amino acid sequence that is identical to SEQ ID NO: 6.

In some embodiments, the isolated antibody comprises a light chain CDR1 comprising the amino acid sequence QGNLLPRHYAH (SEQ ID NO: 16); light chain CDR2 comprising the amino acid sequence DDNIRPS (SEQ ID NO: 17); and/or light chain CDR3 comprising the amino acid sequence QSADLNDDAV (SEQ ID NO: 19).

In some embodiments, the isolated antibody comprises a heavy chain CDR1 comprising the amino acid sequence YYAWS (SEQ ID NO: 12); a heavy chain CDR2 comprising the amino acid sequence DIANEGSTYYSPSLKS (SEQ ID NO: 15); a heavy chain CDR3 comprising the amino acid sequence LRSLYTDYDPHYYDY (SEQ ID NO: 14); a light chain CDR1 comprising the amino acid sequence QGNLLPRHYAH (SEQ ID NO: 16); a light chain CDR2 comprising the amino acid sequence DDNIRPS (SEQ ID NO: 17); and/or a light chain CDR3 comprising the amino acid sequence QSADLNDDAV (SEQ ID NO: 19).

In some embodiments, the isolated antibody comprises a heavy chain variable region amino acid sequence that is identical to SEQ ID NO: 5; and a light chain variable region amino acid sequence that is identical to SEQ ID NO: 8.

In some embodiments, the isolated antibody comprises a light chain CDR1 comprising the amino acid sequence QGDLLPRHYAH (SEQ ID NO: 9); a light chain CDR2 comprising the amino acid sequence DDDIRPS (SEQ ID NO: 10); and/or a light chain CDR3 comprising the amino acid sequence QSADSNDDAV (SEQ ID NO: 11).

In some embodiments, the isolated antibody comprises a heavy chain CDR1 comprising the amino acid sequence YYAWS (SEQ ID NO: 12); a heavy chain CDR2 comprising the amino acid sequence DIANEGSTYYSPSLES (SEQ ID NO: 20); and/or a heavy chain CDR3 comprising the amino acid sequence LRSLYTDYDPHYYDY (SEQ ID NO: 14); a light chain CDR1 comprising the amino acid sequence QGDLLPRHYAH (SEQ ID NO: 9); a light chain CDR2 comprising the amino acid sequence DDDIRPS (SEQ ID NO: 10); and/or a light chain CDR3 comprising the amino acid sequence QSADSNDDAV (SEQ ID NO: 11).

In some embodiments, the isolated antibody comprises a heavy chain variable region amino acid sequence that is identical to SEQ ID NO: 29; and a light chain variable region amino acid sequence that is identical to SEQ ID NO: 28.

In some embodiments, the isolated antibody comprises a heavy chain CDR1 comprising the amino acid sequence YYAWS (SEQ ID NO: 12); a heavy chain CDR2 comprising the amino acid sequence DIANDGSTYYSPSLES (SEQ ID NO: 13); a heavy chain CDR3 comprising the amino acid sequence LRSLYTDYDPHYYDY (SEQ ID NO: 14); a light chain CDR1 comprising the amino acid sequence QGDLLPRHYAH (SEQ ID NO: 9); a light chain CDR2 comprising the amino acid sequence DDDIRPS (SEQ ID NO: 10); and/or a light chain CDR3 comprising the amino acid sequence QSADSNDDAV (SEQ ID NO: 11).

In some embodiments, the isolated antibody comprises a heavy chain variable region amino acid sequence that is identical to SEQ ID NO: 27; and a light chain variable region amino acid sequence that is identical to SEQ ID NO: 26.

In one aspect, the present invention provides an isolated antibody that specifically binds complement factor D (CFD), wherein the heavy chain CDR1 comprises the amino acid sequence YYAWS (SEQ ID NO: 12); the heavy chain CDR2 comprises the amino acid sequence DIANDGSTYYSPSLES (SEQ ID NO: 13); the heavy chain CDR3 comprises the amino acid sequence LRSLYTDYDPHYYDY (SEQ ID NO: 14); the light chain CDR1 comprises the amino acid sequence QGNLLPRHYAH (SEQ ID NO: 16); the light chain CDR2 comprises the amino acid sequence DDNIRPS (SEQ ID NO: 17); and/or the light chain CDR3 comprises the amino acid sequence QSADSNDDAV (SEQ ID NO: 11).

In some embodiments, the isolated antibody comprises a heavy chain CDR1 comprising the amino acid sequence YYAWS (SEQ ID NO: 12); a heavy chain CDR2 comprising the amino acid sequence DIANDGSTYYSPSLES (SEQ ID NO: 13); a heavy chain CDR3 comprising the amino acid sequence LRSLYTDYDPHYYDY (SEQ ID NO: 14); a light chain CDR1 comprising the amino acid sequence QGNLLPRHYAH (SEQ ID NO: 16); a light chain CDR2 comprising the amino acid sequence DDNIRPS (SEQ ID NO: 17); and/or a light chain CDR3 comprising the amino acid sequence QSADSNDDAV (SEQ ID NO: 11).

In some embodiments, the isolated antibody comprises a light chain CDR1 comprising the amino acid sequence QGNLLPRHYAH (SEQ ID NO: 16); a light chain CDR2 comprising the amino acid sequence DDNIRPS (SEQ ID NO: 17); and/or a light chain CDR3 comprising the amino acid sequence QSADSNDDAV (SEQ ID NO: 11).

In some embodiments, the isolated antibody comprises a heavy chain CDR1 comprising the amino acid sequence YYAWS (SEQ ID NO: 12); a heavy chain CDR2 comprising the amino acid sequence DIANDGSTYYSPSLES (SEQ ID NO: 13); and/or a heavy chain CDR3 comprising the amino acid sequence LRSLYTDYDPHYYDY (SEQ ID NO: 14).

In some embodiments, the isolated antibody comprises a heavy chain variable region amino acid sequence that is identical to SEQ ID NO: 34; and a light chain variable region amino acid sequence that is identical to SEQ ID NO: 35.

In some embodiments, the isolated antibody comprises a heavy chain variable region amino acid sequence that is identical to SEQ ID NO: 36; and a light chain variable region amino acid sequence that is identical to SEQ ID NO: 37.

In some embodiments, the isolated antibody comprises a light chain variable domain having at least 90% identity to the amino acid sequence set forth in SEQ ID NO: 35 or SEQ ID NO: 37; a heavy chain variable domain having at least 90% identity to the amino acid sequence set forth in SEQ ID NO: 34 or SEQ ID NO: 36; and/or a light chain variable domain of (a) and a heavy chain variable domain of (b).

In some embodiments, the isolated antibody comprises a light variable domain having at least 95% identity to the amino acid sequence set forth in SEQ ID NO: 35 or SEQ ID NO: 37.

In some embodiments, the isolated antibody comprises a heavy chain variable domain having at least 95% identity to the amino acid sequence set forth in SEQ ID NO: 34 or SEQ ID NO: 36.

In some embodiments, the isolated antibody is a monoclonal antibody or fragment thereof.

In some embodiments, the isolated antibody further comprises an IgG constant region.

In some embodiments, the isolated antibody comprises a light chain amino acid sequence having at least 85%, 90%, 95%, 98%, 99% or 100% sequence identity to SEQ ID NO: 1, 3, 30 or 32.

In some embodiments, the isolated antibody comprises a heavy chain amino acid sequence having at least 85%, 90%, 95%, 98%, 99% or 100% sequence identity to SEQ ID NO: 2, 4, 31 or 33.

In some embodiments, the isolated antibody inhibits the alternative complement pathway. In some embodiments, the isolated antibody inhibits cleavage of complement factor B.

In some embodiments, the isolated antibody binds to CFD at pH 7.4 with an affinity dissociation constant (KD) of between about 1 pM to about 50 pM.

In some embodiments, the isolated antibody binds to CFD at pH 7.4 with an affinity dissociation constant (KD) of less than about 50 pM, less than about 45 pM, less than about 40 pM, less than about 35 pM, less than about 30 pM, less than about 25 pM, less than about 20 pM, less than about 15 pM, less than about 10 pM, less than about 9 pM, less than about 8 pM, less than about 7 pM, less than about 6 pM, or less than about 5 pM.

In some embodiments, the isolated antibody binds to CFD at pH 5.5 with an affinity dissociation constant (KD) of between about 15 nM to about 150 nM.

In some embodiments, the isolated antibody binds to CFD at pH 5.5 with an affinity dissociation constant (KD) of greater than about 15 nM, greater than about 20 nM, greater than about 25 nM, greater than about 30 nM, greater than about 35 nM, greater than about 40 nM, greater than about 45 nM, greater than about 50 nM, greater than about 100 nM, or greater than about 150 nM.

In some embodiments, the isolated antibody the off rate of CFD from the antibody at pH 5.5 is greater than 0.010 s⁻¹, greater than 0.015 s⁻¹, greater than 0.02 s⁻¹, greater than 0.025 s⁻¹, greater than 0.03 s⁻¹, greater than 0.035 s⁻¹, or greater than 0.04 s⁻¹.

In some embodiments, the isolated antibody has a serum half-life of greater than about 5 days, about 10 days, about 15 days, about 20 days, about 25 days, about 30 days, about 35 days, about 40 days, about 45 days, about 50 days, about 60 days, about 70 days, about 80 days, about 90 days, about 95 days, about 100 days, about 125 days or longer.

In one aspect, the present invention provides a nucleic acid sequence encoding an isolated antibody described herein.

In one aspect, the present invention provides a vector comprising the nucleic acid sequence encoding an isolated antibody described herein.

In one aspect, the present invention provides a host cell comprising the nucleic acid sequence encoding an isolated antibody described herein.

In one aspect, the present invention provides a method of producing an antibody, comprising culturing a host cell of under conditions suitable for expression of the antibody.

In one aspect, the present invention provides an antibody, or antigen binding fragment thereof described herein, for use as a medicament.

In one aspect, the present invention provides a method of treating a complement-mediated disease or disorder, the method comprising administering to a subject in need thereof an effective amount of an antibody described herein.

In some embodiments, the complement mediated disease or disorder is atypical hemolytic uremic syndrome (aHUS) or Paroxysmal Nocturnal Hemoglobinuria (PNH).

In some embodiments, administration of the antibody inhibits intravascular hemolysis and extravascular hemolysis.

### Definitions

A or An: The articles “a” and “an” are used herein to refer to one or to more than one (i.e., to at least one) of the grammatical object of the article. By way of example, “an element” means one element or more than one element.

Affinity: As used herein, the term “affinity” refers to the characteristics of a binding interaction between a binding moiety (e.g., an antigen binding moiety (e.g., variable domain described herein) and/or Fe receptor binding moiety (e.g., FcRn binding moiety described herein)) and a target (e.g., an antigen (e.g., CFD) and/or FcR (e.g., FcRn)) and that indicates the strength of the binding interaction. In some embodiments, the measure of affinity is expressed as a dissociation constant (K_{D}). In some embodiments, a binding moiety has a high affinity for a target (e.g., a K_{D} of less than about 10⁻⁷ M, less than about 10⁻⁸ M, or less than about 10⁻⁹ M). In some embodiments, a binding moiety has a low affinity for a target (e.g., a K_{D} of higher than about 10⁻⁷ M, higher than about 10⁻⁶ M, higher than about 10⁻⁵ M, or higher than about 10⁻⁴ M). In some embodiments, a binding moiety has high affinity for a target at a first pH, has low affinity for the target at a second pH, and has an intermediate affinity for the target at a pH level between the first pH and the second pH.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Antibody: As used herein, the term “antibody” refers to a polypeptide that includes at least one immunoglobulin variable region, e.g., an amino acid sequence that provides an immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab, F(ab′)₂, Fd, Fv, and dAb fragments) as well as complete antibodies, e.g., intact immunoglobulins of types IgA, IgG, IgE, IgD, IgM (as well as subtypes thereof). The light chains of the immunoglobulin can be of types kappa or lambda.

Binding Moiety: As used herein, a “binding moiety” is any molecule or part of a molecule capable of specifically binding a target, e.g., a target of interest (e.g., an antigen (e.g., CFD) and/or FcR (e.g., FcRn)). Binding moieties include, e.g., antibodies, antigen binding fragments thereof, Fc regions or Fc fragments thereof, antibody mimetics, peptides, and aptamers.

Constant region: As used herein, the term “constant region” refers to a polypeptide that corresponds to, or is derived from, one or more constant region immunoglobulin domains of an antibody. A constant region can include any or all of the following immunoglobulin domains: a CH1 domain, a hinge region, a CH2 domain, a CH3 domain (derived from an IgA, IgD, IgG, IgE, or IgM), and a CH4 domain (derived from an IgE or IgM).

Fc region: As used herein, the term “Fc region” refers to a dimer of two “Fc polypeptides”, each “Fc polypeptide” comprising the constant region of an antibody excluding the first constant region immunoglobulin domain. In some embodiments, an “Fc region” includes two Fc polypeptides linked by one or more disulfide bonds, chemical linkers, or peptide linkers. “Fc polypeptide” refers to the last two constant region immunoglobulin domains of IgA, IgD, and IgG, and the last three constant region immunoglobulin domains of IgE and IgM, and may also include part or all of the flexible hinge N-terminal to these domains. For IgG, “Fc polypeptide” comprises immunoglobulin domains Cgamma2 (Cγ2) and Cgamma3 (Cγ3) and the lower part of the hinge between Cgamma1 (Cγ1) and Cγ2. Although the boundaries of the Fc polypeptide may vary, the human IgG heavy chain Fc polypeptide is usually defined to comprise residues starting at T223 or C226 or P230, to its carboxyl-terminus, wherein the numbering is according to the EU index as in Kabat et al. (1991, NIH Publication 91-3242, National Technical Information Services, Springfield, VA). For IgA, Fc polypeptide comprises immunoglobulin domains Calpha2 (Cα2) and Calpha3 (Cα3) and the lower part of the hinge between Calpha1 (Cα1) and Cα2. An Fc region can be synthetic, recombinant, or generated from natural sources such as IVIG.

Kₐ: As used herein, “Kₐ” refers to an association rate of a particular binding moiety and a target to form a binding moiety/target complex.

K_{d}: As used herein, “K_{d}” refers to a dissociation rate of a particular binding moiety/target complex.

K_{D}: As used herein, “K_{D}” refers to a dissociation constant, which is obtained from the ratio of K_{d} to Kₐ (i.e., K_{d}/Kₐ) and is expressed as a molar concentration (M). K_{D} values can be determined using methods well established in the art, e.g., by using surface plasmon resonance, or using a biosensor system such as a Biacore® system.

Reference: A “reference” entity, system, amount, set of conditions, etc., is one against which a test entity, system, amount, set of conditions, etc. is compared as described herein. For example, in some embodiments, a “reference” antibody is a control antibody that is not engineered as described herein.

Selective binding: As used herein, “selective binding”, “selectively binds” “specific binding”, or “specifically binds” refers, with respect to a binding moiety and a target, preferential association of a binding moiety to a target and not to an entity that is not the target. A certain degree of non-specific binding may occur between a binding moiety and a non-target. In some embodiments, a binding moiety selectively binds a target if binding between the binding moiety and the target is greater than 2-fold, greater than 5-fold, greater than 10-fold, or greater than 100-fold as compared with binding of the binding moiety and a non-target. In some embodiments, a binding moiety selectively binds a target if the binding affinity is less than about 10⁻⁵ M, less than about 10⁻⁶ M, less than about 10⁻⁷ M, less than about 10⁻⁸ M, or less than about 10⁻⁹ M.

Subject: The term “subject”, as used herein, means any subject for whom diagnosis, prognosis, or therapy is desired. For example, a subject can be a mammal, e.g., a human or non-human primate (such as an ape, monkey, orangutan, or chimpanzee), a dog, cat, guinea pig, rabbit, rat, mouse, horse, cattle, or cow.

Target: As used herein, a “target” is any molecule specifically bound by a binding moiety of a multi-specific binding molecule. In some embodiments, a target is an antigen described herein (e.g., CFD). In some embodiments, a target is an FcR (e.g., FcRn). The terms “first target” and “second target” are used herein to refer to molecules of two distinct molecular species, rather than two molecules of the same molecular species. For example, in some embodiments, a first target is a serum protein and a second target is FcRn.

Therapeutically effective amount: As used herein, the term “therapeutically effective amount” refers to an amount of a therapeutic molecule (e.g., an engineered antibody described herein) which confers a therapeutic effect on a treated subject, at a reasonable benefit/risk ratio applicable to any medical treatment. The therapeutic effect may be objective (i.e., measurable by some test or marker) or subjective (i.e., subject gives an indication of or feels an effect). In particular, the “therapeutically effective amount” refers to an amount of a therapeutic molecule or composition effective to treat, ameliorate, or prevent a particular disease or condition, or to exhibit a detectable therapeutic or preventative effect, such as by ameliorating symptoms associated with the disease, preventing or delaying the onset of the disease, and/or also lessening the severity or frequency of symptoms of the disease. A therapeutically effective amount can be administered in a dosing regimen that may comprise multiple unit doses. For any particular therapeutic molecule, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular subject may depend upon a variety of factors including the disorder being treated and the severity of the disorder; the activity of the specific pharmaceutical agent employed; the specific composition employed; the age, body weight, general health, sex and diet of the subject; the time of administration, route of administration, and/or rate of excretion or metabolism of the specific therapeutic molecule employed; the duration of the treatment; and like factors as is well known in the medical arts.

Treatment: As used herein, the term “treatment” (also “treat” or “treating”) refers to any administration of a therapeutic molecule (e.g., an engineered antibody described herein) that partially or completely alleviates, ameliorates, relieves, inhibits, delays onset of, reduces severity of and/or reduces incidence of one or more symptoms or features of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition.

### BRIEF DESCRIPTION OF DRAWINGS

Drawings are for illustration purposes only; not for limitation.

FIG. 1 is a schematic illustrating alternative pathway activation in PNH patients leading to hemolysis and the anti-CFD strategy.

FIG. 2A is a schematic illustrating in vitro assays for evaluation of potency of anti-CFD mAb inhibition. Hemolysis levels can be measured by optical density (OD) from released hemoglobin. MAC levels can be measured using complement activation in a terminal complement complex (TCC) kit.

FIG. 2B shows exemplary results of a terminal complement complex (TCC) assay to measure membrane attack complex (MAC) formation in the presence of two anti-CFD antibodies. CFD depleted serum doesn't show any alternative pathway activity.

FIG. 3 shows exemplary percent maximum geometric mean of C3 deposition on RBCs using a hemolysis assay. Anti-CFD antibody 2 (169C8) inhibits C3b deposition on rabbit RBC similar to Anti-CFD antibody 1 (Benchmark anti-CFD antibody) compared to no antibody Control (left).

FIG. 4 shows Biacore sensogram of exemplary anti-CFD antibody 2 binding affinity to human CFD and cyno CFD.

FIG. 5 shows exemplary concentration of anti-CFD antibody 2 (clone 169C8) (μg/ml) over time in knock-in human FcRn mice.

FIG. 6 shows exemplary levels of total hIgG (μg/ml) in animals injected with anti-CFD mAbs over time in non human primates (NHP).

FIG. 7 shows exemplary percent TCC formation over time with intravenously (IV) or subcutaneously (SC) administered anti-CFD antibody in NHP.

FIG. 8 shows exemplary levels of total hIgG (μg/ml) in animals injected subcutaneously with anti-CFD mAbs over time, concentration of free CFD, and percent TCC formation over time in non human primates (Top). The free target level after anti-CFD injection (middle). Duration of CFD inhibition after anti-CFD antibody injection (bottom).

FIG. 9 shows exemplary total hIgG (μg/ml) over time in NHP with multiple injections indicated with arrows.

FIG. 10 shows percent TCC formation over time in NHP with multiple injections indicated with arrows.

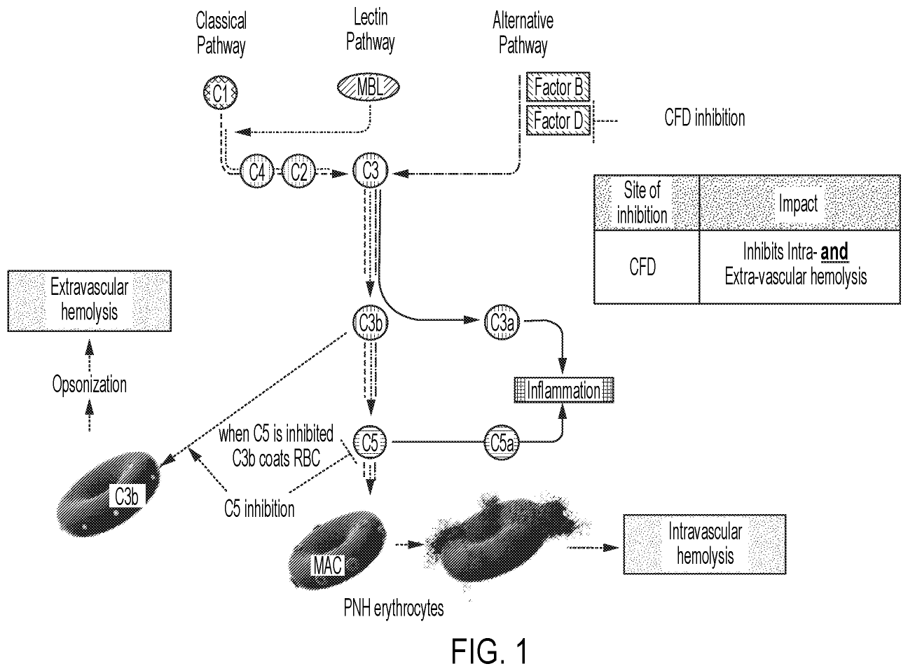

### DETAILED DESCRIPTION

Inhibition of Complement Factor D may prevent Alternative Pathway activation. The present disclosure is based, in part, on the discovery of engineered antibodies that surprisingly exhibit pH-dependent binding to CFD (e.g., human CFD) and/or altered (e.g., increased, e.g., pH dependent) binding to Fc receptor (e.g., FcRn) resulting in depletion of CFD. CFD inhibition using antibodies described herein, blocks intravascular and extravascular hemolysis.

### Complement Factor D

Complement factor D (CFD), also known as adipsin, is a serine protease that is indispensable for the initiation the alternative pathway of the complement system. Human complement factor D is synthesized as a 253 amino acid precursor that contains a signal peptide (aa 1-20), a five-residue activation/pro-peptide (aa 21-25), and the mature chain (aa 26-253). Mature human factor D shares 98% aa sequence identity with the chimpanzee, 96% with rhesus monkey, 84% with porcine, 66% with mouse factor D. Factor D is expressed in multiple tissues including monocyte/macrophages, muscle, sciatic nerve, endometrium, kidney, intestine, and at especially high levels in adipocytes.

Aberrant activation of the alternative complement pathway may cause complement mediated destruction of red blood cells (RBCs). For example, CD 55/59 deficiency on the surface of red blood cells (RBCs) leads to terminal complement-mediated deposition and destruction of susceptible RBCs. Sequestered oxyhemoglobin may cause hemolysis releasing free hemoglobin resulting in nitric oxide depletion. Renal toxicity results from free hemoglobin iron and hemosiderin deposition from the lysed red blood cell. Repeated exposure to toxic substances, thromboses and necrosis can result in renal failure. Hemolyisis may also cause complications including renal failure, pulmonary hypertension and cholelithiasis.

### Antibodies

Anti-CFD antibodies described herein are designed to inhibit complement factor D (CFD) activity by inhibition of factor B cleavage in the alternative pathway. This inhibition activity blocks intra and extra-vascular hemolysis. The anti-CFD antibodies according to the present invention demonstrate species cross-reactivity to human and cynomolgus monkey (cyno) for both acid-switching half-life extension (ASHE) and CFD inhibition. In some embodiments, anti-CFD antibodies are engineered to bind CFD with higher affinity at pH 7.4 than at pH 5.5. In some embodiments the antibody binding at pH 7.4, is less than or equal to ≤20 pM. In some embodiments, CFD is released at acidic pH 5.5: kd>20 nM, off rate of >10E⁻² s⁻¹. In some embodiments, the anti-CFD antibody has a half-life of approximately 20 days in cyno or >45 days in knock-in huFcRn mice.

An engineered antibody described herein can be an immunoglobulin, heavy chain antibody, light chain antibody, LRR-based antibody, or other protein scaffold with antibody-like properties, as well as other immunological binding moiety known in the art, including, e.g., a Fab, Fab′, Fab′2, Fab₂, Fab₃, F(ab′)₂, Fd, Fv, Feb, scFv, SMIP, antibody, diabody, triabody, tetrabody, minibody, maxibody, tandab, DVD, BiTe, TandAb, or the like, or any combination thereof. The subunit structures and three-dimensional configurations of different classes of antibodies are known in the art.

An antibody can be an immunoglobulin molecule of four polypeptide chains, e.g., two heavy (H) chains and two light (L) chains. A heavy chain can include a heavy chain variable domain and a heavy chain constant domain. A heavy chain constant domain can include CH1, hinge, CH2, CH3, and in some instances CH4 regions. A suitable heavy chain constant region may be derived from any immunoglobulin (e.g., IgA, IgG, or IgE). In some embodiments, a suitable heavy chain constant region may be derived from IgG1, IgG2, or IgG4. In particular embodiments, a suitable heavy chain constant region is derived from IgG1. A light chain can include a light chain variable domain and a light chain constant domain. A light chain constant domain can include either a kappa light chain or a lambda light chain. A heavy chain variable domain of a heavy chain and a light chain variable domain of a light chain can typically be further subdivided into regions of variability, termed complementarity determining regions (CDRs), interspersed with regions that are more conserved, termed framework regions (FR). Such heavy chain and light chain variable domains can each include three CDRs and four framework regions, arranged from amino-terminus to carboxyl-terminus in the following order: FR1, CDR1, FR2, CDR2, FR3, CDR3, FR4, one or more of which can be engineered as described herein.

### Engineered Heavy Chain

In some embodiments, engineered antibodies described herein can include a heavy chain comprising or consisting of the amino acid sequence of SE ID NO: 2.

(SEQ ID NO: 2)
EVQVQESGPGLVKPSQTLSLTCTVSGGSITTSYYAWSWIRQPPGKGLEW
MGDIANDGSTYYSPSLESRTSISRDTSKNQFSLQLSSVTPEDTAVYYCA
RLRSLYTDYDPHYYDYWGQGTQVTVSSASTKGPSVFPLAPSSKSTSGGT
AALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPEAAGG
PSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKT
ISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALK
FHYTQKSLSLSPGK.

In some embodiments, engineered antibodies described herein can include a heavy chain variable region comprising or consisting of the amino acid sequence of SEQ ID NO: 27.

(SEQ ID NO: 27)
EVQVQESGPGLVKPSQTLSLTCTVSGGSITTSYYAWSWIRQPPGKGLEW
MGDIANDGSTYYSPSLESRTSISRDTSKNQFSLQLSSVTPEDTAVYYCA
RLRSLYTDYDPHYYDYWGQGTQVTVSS

In some embodiments, engineered antibodies described herein can include a heavy chain comprising or consisting of the amino acid sequence of SEQ ID NO: 4.

(SEQ ID NO: 4)
QVQLQESGPGLVKPSQTLSLTCTVSGGSISTSYYAWSWIRQPPGKGLEW
IGDIANEGSTYYSPSLESRVTISRDTSKNQFSLQLSSVTAADTAVYYCA
RLRSLYTDYDPHYYDYWGQGTLVTVSSASTKGPSVFPLAPSSKSTSGGT
AALGCLVKDYFPEPVTVSWNSGALTSGVHTFPAVLQSSGLYSLSSVVTV
PSSSLGTQTYICNVNHKPSNTKVDKRVEPKSCDKTHTCPPCPAPEAAGG
PSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKT
ISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALK
FHYTQKSLSLSPG

In some embodiments, engineered antibodies described herein can include a heavy chain comprising or consisting of the amino acid sequence of SEQ ID NO: 31.

(SEQ ID NO: 31)
QVQVQESGPGLVKPSQTLSLTCTVSGGSISTSYYAWSWIRQPPGKGLEW
MGDIANDGSTYYSPSLESRVTISRDTSKNQFSLQLSSVTAQDTAVYYCA
RLRSLYTDYDPHYYDYWGQGTLVTVSSASTKGPSVFPLAPSSKSTSGGT
AALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPEAAGG
PSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKT
ISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALK
FHYTQKSLSLSPG

In some embodiments, engineered antibodies described herein can include a heavy chain comprising or consisting of the amino acid sequence of SEQ ID NO: 33.

(SEQ ID NO: 33)
QVQVQESGPGLVKPSQTLSLTCTVSGGSISTSYYAWSWIRQPPGKGLEW
MGDIANDGSTYYSPSLESRVTISRDTSKNQFSLQLSSVTAQDTAVYYCA
RLRSLYTDYDPHYYDYWGQGTQVTVSSASTKGPSVFPLAPSSKSTSGGT
AALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPEAAGG
PSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKT
ISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALK
FHYTQKSLSLSPG

In some embodiments, the engineered antibody comprises a heavy chain amino acid sequence having at least 85%, 90%, 95%, 98%, or 99% sequence identity to SEQ ID NO: 2, 4, 31 or 33. In some embodiments, the engineered antibodies comprise a heavy chain amino acid sequence identical to SEQ ID NO: 2, 4, 31 or 33. In some embodiments, the engineered antibody comprises no more than 30, 29, 28, 27, 26, 25, 24, 23, 22, 21, 20, 19, 18, 17, 16, 15, 14, 13, 12, 11, 10, 9, 8, 7, 6, 5, 4, 3, or 2 amino acid substitutions relative to SEQ ID NO: 2, 4, 31 or 33. In some embodiments, the engineered antibody comprises a heavy chain amino acid sequence with one or more amino acid substitutions at one or more of positions 1, 4, 30, 50, 56, 69, 70, 89, 90, 120, 222, 364, or 366 relative to SEQ ID NO: 2, 4, 31 or 33.

In some embodiments, engineered antibodies described herein can include a heavy chain variable region comprising or consisting of the amino acid sequence of SEQ ID NO: 29.

(SEQ ID NO: 29)
QVQLQESGPGLVKPSQTLSLTCTVSGGSISTSYYAWSWIRQPPGKGLEW
IGDIANEGSTYYSPSLESRVTISRDTSKNQFSLQLSSVTAADTAVYYCA
RLRSLYTDYDPHYYDYWGQGTLVTVSS

In some embodiments, engineered antibodies described herein can include a heavy chain variable region comprising or consisting of the amino acid sequence of SEQ ID NO:5.

(SEQ ID NO: 5)
QVQLQESGPGLVKPSQTLSLTCTVSGGSISTSYYAWSWIRQPPGKGLEW
IGDIANEGSTYYSPSLKSRVTISRDTSKNQFSLQLSSVTAADTAVYYCA
RLRSLYTDYDPHYYDYWGQGTLVTVSS

In some embodiments, engineered antibodies described herein can include a heavy chain variable region comprising or consisting of the amino acid sequence of SEQ ID NO: 34.

(SEQ ID NO: 34)
QVQVQESGPGLVKPSQTLSLTCTVSGGSISTSYYAWSWIRQPPGKGLEW
MGDIANDGSTYYSPSLESRVTISRDTSKNQFSLQLSSVTAQDTAVYYCA
RLRSLYTDYDPHYYDYWGQGTLVTVSS

In some embodiments, engineered antibodies described herein can include a heavy chain variable region comprising or consisting of the amino acid sequence of SEQ ID NO: 36.

(SEQ ID NO: 36)
QVQVQESGPGLVKPSQTLSLTCTVSGGSISTSYYAWSWIRQPPGKGLEW
MGDIANDGSTYYSPSLESRVTISRDTSKNQFSLQLSSVTAQDTAVYYCA
RLRSLYTDYDPHYYDYWGQGTQVTVSS

In some embodiments, the engineered antibody comprises a heavy chain amino acid sequence having at least 85%, 90%, 95%, 98%, or 99% sequence identity to SEQ ID NO: 5, 27, 29, 34 or 36. In some embodiments, the engineered antibodies comprise a heavy chain amino acid sequence identical to SEQ ID NO: 5, 27, 29, 34 or 36.

In some embodiments, the engineered antibody comprises a heavy chain amino acid sequence having at least 85%, 90%, 95%, 98%, or 99% sequence identity to SEQ ID NO: 5, 27, 29, 34 or 36. In some embodiments, the engineered antibodies comprise a heavy chain amino acid sequence identical to SEQ ID NO: 5, 27, 29, 34 or 36. In some embodiments, the engineered antibody comprises no more than 30, 29, 28, 27, 26, 25, 24, 23, 22, 21, 20, 19, 18, 17, 16, 15, 14, 13, 12, 11, 10, 9, 8, 7, 6, 5, 4, 3, or 2 amino acid substitutions relative to SEQ ID NO: 5, 27, 29, 34 or 36. In some embodiments, the engineered antibody comprises a heavy chain amino acid sequence with one or more amino acid substitutions at one or more of positions 1, 4, 30, 50, 56, 69, 70, 89, 90 120, or 222 relative to SEQ ID NO: 5, 27, 29, 34 or 36.

In some embodiments, the engineered antibodies described herein comprise a heavy chain CDR1 comprises the amino acid sequence YYAWS (SEQ ID NO: 12); a heavy chain CDR2 comprises the amino acid sequence DIANEGSTYYSPSLKS (SEQ ID NO: 15); and/or a heavy chain CDR3 comprises the amino acid sequence LRSLYTDYDPHYYDY (SEQ ID NO: 14).

In some embodiments, the engineered antibodies described herein comprise a heavy chain CDR1 comprises the amino acid sequence YYAWS (SEQ ID NO: 12); a heavy chain CDR2 comprises the amino acid sequence DIANEGSTYYSPSLES (SEQ ID NO: 20); and/or a heavy chain CDR3 comprises the amino acid sequence LRSLYTDYDPHYYDY (SEQ ID NO: 14).

In some embodiments, the engineered antibodies described herein comprise a heavy chain CDR1 comprises the amino acid sequence YYAWS (SEQ ID NO: 12); a heavy chain CDR2 comprises the amino acid sequence DIANDGSTYYSPSLES (SEQ ID NO: 13); and/or a heavy chain CDR3 comprises the amino acid sequence LRSLYTDYDPHYYDY (SEQ ID NO: 14).

As will be understood by those of skill in the art, any such heavy chain CDR sequence may be readily combined, e.g., by techniques of molecular biology, with any other antibody sequences or domains provided herein or otherwise known in the art, including any framework regions, CDRs, or constant domains, or portions thereof as disclosed herein or otherwise known in the art, as may be present in an antibody or binding molecule of any format as disclosed herein or otherwise known in the art.

### Engineered Heavy Chain Constant Domains

In various engineered antibodies described herein, a heavy chain constant domain can be of any class (or subclass). In various engineered antibodies described herein, a heavy chain constant domain can include the amino acid sequence of any of one or more of IgG, IgM, IgA, IgD, or IgE, including subclasses such as IgG1, IgG2, IgG3, IgG4, IgA1, and IgA2. In various instances, a constant domain of engineered antibodies described herein can include a mixture of two or more classes (or subclasses) of immunoglobulin heavy chain constant domain. For instance, an engineered antibody can include a first portion of a constant domain that has a sequence of an immunoglobulin constant domain selected from an IgG, IgM, IgA, IgD, or IgE class constant domain and a second portion of a constant domain that has a sequence of an immunoglobulin constant domain different from the first and selected from an IgG, IgM, IgA, IgD, or IgE class constant domain. In some instances, a constant domain of an engineered antibody described herein can include a mixture of two or more subclasses of a particular class of constant domain, e.g., a first portion of a constant domain that has a sequence of an immunoglobulin constant domain selected from an IgG1, IgG2, IgG3, or IgG4 subclass constant domain and a second portion of a constant domain that has a sequence of an immunoglobulin constant domain different from the first and selected from an IgG1, IgG2, IgG3, or IgG4 subclass constant domain. In some particular embodiments, a constant domain includes all or a portion of an IgG2 constant domain and all or a portion of an IgG4 constant domain.

In some instances, an engineered antibody includes an antibody constant region, Fc region or Fc fragment that exhibits altered binding (as compared to a reference constant region) to one or more Fc receptors (e.g., FcγRI, FcγRIIA, FcγRIIB, FcγRIIIA, FcγRIIIB, FcγRIV, or FcRn receptor). In some embodiments, a constant region, Fc region or Fc fragment is engineered to bind to a target (e.g., an FcRn receptor) in an altered manner (e.g., in a pH sensitive manner (e.g., in a more or less pH sensitive manner) and/or decreased or increased binding) relative to a reference constant region, Fc region or Fc fragment. In some embodiments, an engineered antibody includes an antibody constant region, Fc region or Fc fragment that exhibits decreased binding (as compared to a reference constant region) to one or more Fcγ receptor (e.g., FcγRI, FcγRIIA, FcγRIIB, FcγRIIIA, FcγRIIIB, or FcγRIV). In some embodiments, engineered antibody includes an antibody constant region, Fc region or Fc fragment that exhibits increased binding to the FcRn receptor (as compared to a reference constant region) at serum pH and/or at intracellular pH.

For example, an engineered antibody can include a constant region, Fc region or Fc fragment of an IgG antibody engineered to include an amino acid addition, deletion, or substitution, of one or more of amino acid residues 251-256, 285-290, 308-314, 385-389, and 428-436 (Kabat numbering (Kabat et al., (1991) Sequences of Proteins of Immunological Interest, NIH)). Without wishing to be bound by theory, it is believed that one or more of these constant regions, Fc region, or Fc fragment amino acids mediate interaction with an Fc receptor, e.g., FcRn. In some embodiments, one or more of these disclosed amino acids is substituted with histidine, arginine, lysine, aspartic acid, glutamic acid, serine, threonine, asparagine, or glutamine. In some embodiments, a non-histidine residue is substituted with a histidine residue. In some embodiments, a histidine residue is substituted with a non-histidine residue.

In some embodiments, an engineered antibody includes a constant region, Fc region or Fc fragment of an IgG antibody having amino acid modifications at one or more of positions 308, 309, 311, 312, and 314, more specifically, having substitutions at one or more of positions 308, 309, 311, 312 and 314 with threonine, proline, serine, aspartic acid and leucine respectively. In some embodiments, residues at one or more of positions 308, 309, and 311 are substituted with isoleucine, proline, and glutamic acid, respectively. In yet other embodiments, residues at one or more of positions 308, 309, 311, 312, and 314, are substituted with threonine, proline, serine, aspartic acid, and leucine, respectively.

In some embodiments, an engineered antibody includes a constant region, Fc region or Fc fragment of an IgG antibody having amino acid modifications at one or more of positions 251, 252, 254, 255, and 256, more specifically, having substitutions at one or more of these positions. In some embodiments, residue 251 is substituted with leucine or arginine, residue 252 is substituted with leucine, tyrosine, phenylalanine, serine, tryptophan or threonine, residue 254 is substituted with threonine or serine, residue 255 is substituted with leucine, glycine, isoleucine or arginine, and/or residue 256 is substituted with serine, phenylalanine, arginine, glutamine, glutamic acid, aspartic acid, alanine, asparagine or threonine. In some embodiments, residue 251 is substituted with leucine, residue 252 is substituted with tyrosine or leucine, residue 254 is substituted with threonine or serine, and/or residue 255 is substituted with arginine. In yet other embodiments, residue 252 is substituted with phenylalanine and/or residue 256 is substituted with aspartic acid. In some embodiments, residue 251 is substituted with leucine, residue 252 is substituted with tyrosine, residue 254 is substituted with threonine or serine, and/or residue 255 is substituted with arginine.

In some embodiments, an engineered antibody includes a constant region, Fc region or Fc fragment of an IgG antibody having amino acid modifications at one or more of positions 428, 433, 434, 435, and 436, more specifically, having substitutions at one or more of these positions. In some embodiments, residue 428 is substituted with methionine, threonine, leucine, phenylalanine, or serine, residue 433 is substituted with lysine, arginine, serine, isoleucine, proline, glutamine, or histidine, residue 434 is substituted with phenylalanine, tyrosine, or histidine, residue 435 is substituted with tyrosine, and/or residue 436 is substituted with histidine, asparagine, arginine, threonine, lysine, methionine, or threonine. In some embodiments, residues at one or more positions 433, 434, 435, and 436 are substituted with lysine, phenylalanine, tyrosine, and histidine, respectively. In some embodiments, residue 428 is substituted with methionine and/or residue 434 is substituted with tyrosine.

In some embodiments, an engineered antibody includes a constant region, Fc region or Fc fragment of an IgG antibody having amino acid modifications at one or more of positions 385, 386, 387, and 389, more specifically, having substitutions at one or more of these positions. In some embodiments, residue 385 is substituted with arginine, aspartic acid, serine, threonine, histidine, lysine, or alanine, residue 386 is substituted with threonine, proline, aspartic acid, serine, lysine, arginine, isoleucine, or methionine, residue 387 is substituted with arginine, histidine, serine, threonine, alanine, or proline and/or residue 389 is substituted with proline or serine. In some embodiments, residues at one or more of positions 385, 386, 387, and 389 are substituted with arginine, threonine, arginine, and proline, respectively. In some embodiments, residues at one or more of positions 385, 386, and 389 are substituted with aspartic acid, proline, and serine, respectively.

In some embodiments, an engineered antibody includes a constant region, Fc region or Fc fragment of an IgG antibody having one or more of the following substitutions: leucine at residue 251, tyrosine or leucine at residue 252, threonine or serine at residue 254, arginine at residue 255, threonine at residue 308, proline at residue 309, serine at residue 311, aspartic acid at residue 312, leucine at residue 314, arginine at residue 385, threonine at residue 386, arginine at residue 387, proline at residue 389, methionine at residue 428, lysine at residue 433, phenylalanine or tyrosine at residue 434, tyrosine at position 435, and/or tyrosine at position 436. Additional amino acid substitutions that can be included in a constant region, Fc region or Fc fragment include those described in, e.g., U.S. Pat. Nos. 6,277,375; 8,012,476; and 8,163,881.

In some embodiments, an engineered antibody described herein includes a heavy chain constant domain that include the Ala-Ala mutation described in, e.g., PCT Publication nos. WO 94/28027 and WO 98/47531; and Xu et al. (2000) Cell Immunol 200:16-26. Thus, in some embodiments, an engineered antibody with one or more mutations within the heavy chain constant region including the Ala-Ala mutation has reduced or no effector function. According to these embodiments, the constant region of an engineered antibody described herein can comprise a substitution to an alanine at position 234 and/or a mutation to an alanine at position 235 (EU numbering).

As will be understood by those of skill in the art, any such heavy chain constant domain sequence may be readily combined, e.g., by techniques of molecular biology, with any other antibody sequences or domains provided herein or otherwise known in the art, including any framework regions, CDRs, or constant domains, or portions thereof as disclosed herein or otherwise known in the art, as may be present in an antibody or binding molecule of any format as disclosed herein or otherwise known in the art.

### Engineered Light Chain

In some embodiments, engineered antibodies described herein can include a light chain comprising or consisting of the amino acid sequence of SEQ ID NO: 1.

(SEQ ID NO: 1)
SSALTQPSALSVTKGQTAKITCQGDLLPRHYAHWYQQKTGQAPKLIVYD
DDIRPSGIPERFSGSNSGGVATLTIAGAQAEDEADYHCQSADSNDDAVF
GGGTHLTVLSQPKAAPSVTLFPPSSEELQANKATLVCLISDFYPGAVTV
AWKADSSPVKAGVETTTPSKQSNNKYAASSYLSLTPEQWKSHRSYSCQV
THEGSTVEKTVAPTECS

In some embodiments, engineered antibodies described herein can include a light chain comprising or consisting of the amino acid sequence of SEQ ID NO: 3.

(SEQ ID NO: 3)
SYELTQPPALSVSPGQTARITCQGDLLPRHYAHWYQQKTGQAPKLVIYD
DDIRPSGIPERFSGSSSGTMATLTISGAQAEDEADYHCQSADSNDDAVF
GGGTQLTVLGQPKAAPSVTLFPPSSEELQANKATLVCLISDFYPGAVTV
AWKADSSPVKAGVETTTPSKQSNNKYAASSYLSLTPEQWKSHRSYSCQV
THEGSTVEKTVAPTECS

In some embodiments, engineered antibodies described herein can include a light chain comprising or consisting of the amino acid sequence of SEQ ID NO: 30.

(SEQ ID NO: 30)
SSALTQPPALSVSKGQTARITCQGNLLPRHYAHWYQQKTGQAPKLIVYD
DNIRPSGIPERFSGSNSGGVATLTISGAQAEDEADYHCQSADSNDDAVF
GGGTQLTVLGQPKAAPSVTLFPPSSEELQANKATLVCLISDFYPGAVTV
AWKADSSPVKAGVETTTPSKQSNNKYAASSYLSLTPEQWKSHRSYSCQV
THEGSTVEKTVAPTECS

In some embodiments, engineered antibodies described herein can include a light chain comprising or consisting of the amino acid sequence of SEQ ID NO: 32.

(SEQ ID NO: 32)
SSALTQPSALSVSKGQTARITCQGNLLPRHYAHWYQQKTGQAPKLIVYD
DNIRPSGIPERFSGSNSGGVATLTISGAQAEDEADYHCQSADSNDDAVF
GGGTHLTVLGQPKAAPSVTLFPPSSEELQANKATLVCLISDFYPGAVTV
AWKADSSPVKAGVETTTPSKQSNNKYAASSYLSLTPEQWKSHRSYSCQV
THEGSTVEKTVAPTECS

In some embodiments, the engineered antibody comprises a light chain amino acid sequence having at least 85%, 90%, 95%, 98%, or 99% sequence identity to SEQ ID NO: 1, 3, 30 or 32. In some embodiments, the engineered antibody comprises a light chain amino acid sequence identical to SEQ ID NO: 1, 3, 30 or 32. In some embodiments, the engineered antibody comprises no more than 30, 29, 28, 27, 26, 25, 24, 23, 22, 21, 20, 19, 18, 17, 16, 15, 14, 13, 12, 11, 10, 9, 8, 7, 6, 5, 4, 3, or 2 amino acid substitutions relative to SEQ ID NO: 1, 3, 30 or 32. In some embodiments, the engineered antibody comprises a light chain amino acid sequence with one or more amino acid substitutions at one or more of positions 1, 2, 3, 4, 8, 13, 14, 19, 46, 47, 65, 68, 69, 75, 103, 108 relative to SEQ ID NO: 1, 3, 30 or 32.

In some embodiments, engineered antibodies described herein can include a light chain variable region comprising or consisting of the amino acid sequence of SEQ ID NO: 26.

(SEQ ID NO: 26)
SSALTQPSALSVTKGQTAKITCQGDLLPRHYAHWYQQKTGQAPKLIVYD
DDIRPSGIPERFSGSNSGGVATLTIAGAQAEDEADYHCQSADSNDDAVF
GGGTHLTVLSQPKAAPSVTLFPPSS

In some embodiments, engineered antibodies described herein can include a light chain variable region comprising or consisting of the amino acid sequence of SEQ ID NO: 28.

(SEQ ID NO: 28)
SYELTQPPALSVSPGQTARITCQGDLLPRHYAHWYQQKTGQAPKLVIYD
DDIRPSGIPERFSGSSSGTMATLTISGAQAEDEADYHCQSADSNDDAVF
GGGTQLTVLGQPKAAPSVTL

In some embodiments, engineered antibodies described herein can include a light chain variable region comprising or consisting of the amino acid sequence of SEQ ID NO: 6.

(SEQ ID NO: 6)
SYELTQPPALSVSPGQTARITCQGNLLPRHYAHWYQQKTGQAPKLVIYD
DNIRPSGIPERFSGSSSGTTATLTISGAQAEDEADYHCQSASSNDDAVF
GGGTQLTVL

In some embodiments, engineered antibodies described herein can include a light chain variable region comprising or consisting of the amino acid sequence of SEQ ID NO: 8.

(SEQ ID NO: 8)
SYELTQPPALSVSPGQTARITCQGNLLPRHYAHWYQQKTGQAPKLVIYD
DNIRPSGIPERFSGSSSGTTATLTISGAQAEDEADYHCQSADLNDDAVF
GGGTQLTVL

In some embodiments, engineered antibodies described herein can include a light chain variable region comprising or consisting of the amino acid sequence of SEQ ID NO: 35.

(SEQ ID NO: 35)
SSALTQPPALSVSKGQTARITCQGNLLPRHYAHWYQQKTGQAPKLIVYD
DNIRPSGIPERFSGSNSGGVATLTISGAQAEDEADYHCQSADSNDDAVF
GGGTQLTVL

In some embodiments, engineered antibodies described herein can include a light chain variable region comprising or consisting of the amino acid sequence of SEQ ID NO: 37.

(SEQ ID NO: 37)
SSALTQPSALSVSKGQTARITCQGNLLPRHYAHWYQQKTGQAPKLIVYD
DNIRPSGIPERFSGSNSGGVATLTISGAQAEDEADYHCQSADSNDDAVF
GGGTHLTVL

In some embodiments, the engineered antibody comprises a light chain variable region amino acid sequence having at least 85%, 90%, 95%, 98%, or 99% sequence identity to SEQ ID NO: 6, 8, 26, 28, 35 or 37. In some embodiments, the engineered antibody comprises a light chain variable region amino acid sequence identical to SEQ ID NO: 6, 8, 26, 28, 35 or 37. In some embodiments, the engineered antibody comprises no more than 30, 29, 28, 27, 26, 25, 24, 23, 22, 21, 20, 19, 18, 17, 16, 15, 14, 13, 12, 11, 10, 9, 8, 7, 6, 5, 4, 3, or 2 amino acid substitutions relative to SEQ ID NO:6, 8, 26, 28, 35 or 37. In some embodiments, the engineered antibody comprises a light chain variable region amino acid sequence with one or more amino acid substitutions at one or more of positions 1, 2, 3, 4, 8, 13, 14, 19, 46, 47, 65, 68, 69, 75, 103, 108 relative to SEQ ID NO: 6, 8, 26, 28, 35 or 37.

In some embodiments, the engineered antibodies described herein comprise a light chain CDR1 comprising the amino acid sequence QGNLLPRHYAH (SEQ ID NO: 16), a light chain CDR2 comprising the amino acid sequence DDNIRPS (SEQ ID NO: 17); and/or a light chain CDR3 comprising the amino acid sequence QSASSNDDAV (SEQ ID NO: 18).

In some embodiments, the engineered antibodies described herein comprise a light chain CDR1 comprising the amino acid sequence QGNLLPRHYAH (SEQ ID NO: 16), a light chain CDR2 comprising the amino acid sequence DDNIRPS (SEQ ID NO: 17), and/or light chain CDR3 comprising the amino acid sequence QSADLNDDAV (SEQ ID NO: 19).

In some embodiments, the engineered antibodies described herein comprise a light chain CDR1 comprising the amino acid sequence QGDLLPRHYAH (SEQ ID NO: 9), a light chain CDR2 comprising the amino acid sequence DDDIRPS (SEQ ID NO: 10), and/or a light chain CDR3 comprising the amino acid sequence QSADSNDDAV (SEQ ID NO: 11).

In some embodiments, the engineered antibodies described herein comprise a light chain CDR1 comprising the amino acid sequence QGNLLPRHYAH (SEQ ID NO: 16), a light chain CDR2 comprising the amino acid sequence DDNIRPS (SEQ ID NO: 17), and/or a light chain CDR3 comprising the amino acid sequence QSADSNDDAV (SEQ ID NO: 11).

As will be understood by those of skill in the art, any such light chain CDR sequence may be readily combined, e.g., by techniques of molecular biology, with any other antibody sequences or domains provided herein or otherwise known in the art, including any framework regions, CDRs, or constant domains, or portions thereof as disclosed herein or otherwise known in the art, as may be present in an antibody or binding molecule of any format as disclosed herein or otherwise known in the art.

### Engineered Light Chain Constant Domains

In some embodiments, an engineered antibody described herein includes a light chain that includes any light chain constant domain sequence, e.g., a constant sequence of a light chain known to those of skill in the art. As those of skill in the art will be aware, a light chain constant domain may be a kappa light chain constant domain or a lambda light chain constant domain. In certain embodiments, the constant domain of a light chain as disclosed herein is a kappa light chain constant domain.

In various instances, an engineered antibody described herein includes a light chain constant domain comprising or consisting of the amino acid sequence of SEQ ID NO:434. As will be understood by those of skill in the art, any such light chain constant domain sequence may be readily combined, e.g., by techniques of molecular biology, with any other antibody sequences or domains provided herein or otherwise known in the art, including any framework regions, CDRs, or constant domains, or portions thereof as disclosed herein or otherwise known in the art, as may be present in an antibody or binding molecule of any format as disclosed herein or otherwise known in the art.

### Exemplary Engineered Antibodies

Engineered antibodies can include various heavy chains and light chains described herein. In some embodiments, an engineered antibody can include two heavy chains and light chains. In various instances, the present disclosure encompasses an antibody including at least one heavy chain and/or light chain as disclosed herein, at least one heavy chain and/or light chain framework domain as disclosed herein, at least one heavy chain and/or light chain CDR domain as disclosed herein, and/or any heavy chain and/or light chain constant domain as disclosed herein.

In various instances, an engineered antibody disclosed herein is a homodimeric monoclonal antibody. In various instances, an engineered antibody disclosed herein is a heterodimeric antibody. In various instances, an engineered antibody is, e.g., a typical antibody or a diabody, triabody, tetrabody, minibody, maxibody, tandab, DVD, BiTe, scFv, TandAb scFv, Fab, Fab₂, Fab₃, F(ab′)₂, or the like, or any combination thereof.

In some embodiments, the engineered isolated antibody described herein includes at least one light chain variable domain comprising or consisting of an amino acid sequence having at least 70%, at least 75%, at least 80%, at least 85%, at least 90%, at least 91%, at least 92%, at least 93%, at least 94%, at least 95%, at least 96%, at least 97%, at least 98%, or at least 99% homology or identity to the amino acid sequence of any one of SEQ ID Nos 6, 8, 26, 28, 35 or 37.

In some embodiments, the engineered isolated antibody described herein includes at least one heavy chain variable domain comprising or consisting of an amino acid sequence having at least 70%, at least 75%, at least 80%, at least 85%, at least 90%, at least 91%, at least 92%, at least 93%, at least 94%, at least 95%, at least 96%, at least 97%, at least 98%, or at least 99% homology or identity to the amino acid sequence of any one of SEQ ID Nos 5, 27, 29, 34 or 36.

In some embodiments, the engineered isolated antibody described herein comprises a heavy chain CDR1 comprising the amino acid sequence YYAWS (SEQ ID NO: 12); a heavy chain CDR2 comprising the amino acid sequence DIANEGSTYYSPSLKS (SEQ ID NO: 15); a heavy chain CDR3 comprising the amino acid sequence LRSLYTDYDPHYYDY (SEQ ID NO: 14); a light chain CDR1 comprising the amino acid sequence QGNLLPRHYAH (SEQ ID NO: 16); a light chain CDR2 comprising the amino acid sequence DDNIRPS (SEQ ID NO: 17); and a light chain CDR3 comprising the amino acid sequence QSASSNDDAV (SEQ ID NO: 18).

In some embodiments, the isolated antibody comprises a heavy chain variable region amino acid sequence that is identical to SEQ ID NO: 5; and a light chain variable region amino acid sequence that is identical to SEQ ID NO: 6.

In some embodiments, the engineered isolated antibody described herein comprises a heavy chain CDR1 comprising the amino acid sequence YYAWS (SEQ ID NO: 12); a heavy chain CDR2 comprising the amino acid sequence DIANEGSTYYSPSLKS (SEQ ID NO: 15); a heavy chain CDR3 comprising the amino acid sequence LRSLYTDYDPHYYDY (SEQ ID NO: 14); a light chain CDR1 comprises the amino acid sequence QGNLLPRHYAH (SEQ ID NO: 16); a light chain CDR2 comprises the amino acid sequence DDNIRPS (SEQ ID NO: 17); and a light chain CDR3 comprises the amino acid sequence QSADLNDDAV (SEQ ID NO: 19).

In some embodiments, the isolated antibody comprises a heavy chain variable region amino acid sequence that is identical to SEQ ID NO: 5; and a light chain variable region amino acid sequence that is identical to SEQ ID NO: 8.

In some embodiments, the engineered isolated antibody described herein comprises a heavy chain CDR1 comprising the amino acid sequence YYAWS (SEQ ID NO: 12); a heavy chain CDR2 comprising the amino acid sequence DIANEGSTYYSPSLKS (SEQ ID NO: 15); a heavy chain CDR3 comprising the amino acid sequence LRSLYTDYDPHYYDY (SEQ ID NO: 14); a light chain CDR1 comprising the amino acid sequence QGNLLPRHYAH (SEQ ID NO: 16); a light chain CDR2 comprising the amino acid sequence DDNIRPS (SEQ ID NO: 17); and a light chain CDR3 comprising the amino acid sequence QSADLNDDAV (SEQ ID NO: 19).

In some embodiments, the isolated antibody comprises a heavy chain variable region amino acid sequence that is identical to SEQ ID NO: 29; and a light chain variable region amino acid sequence that is identical to SEQ ID NO: 28.

In some embodiments, the engineered isolated antibody described herein comprises a heavy chain CDR1 comprising the amino acid sequence YYAWS (SEQ ID NO: 12); a heavy chain CDR2 comprising the amino acid sequence DIANDGSTYYSPSLES (SEQ ID NO: 13); a heavy chain CDR3 comprising the amino acid sequence LRSLYTDYDPHYYDY (SEQ ID NO: 14); a light chain CDR1 comprising the amino acid sequence QGDLLPRHYAH (SEQ ID NO: 9); a light chain CDR2 comprising the amino acid sequence DDDIRPS (SEQ ID NO: 10); and a light chain CDR3 comprising the amino acid sequence QSADSNDDAV (SEQ ID NO: 11).

In some embodiments, the isolated antibody comprises a heavy chain variable region amino acid sequence that is identical to SEQ ID NO: 27; and a light chain variable region amino acid sequence that is identical to SEQ ID NO: 26.

In some embodiments, the engineered isolated antibody described herein comprises a heavy chain CDR1 comprising the amino acid sequence YYAWS (SEQ ID NO: 12); a heavy chain CDR2 comprising the amino acid sequence DIANDGSTYYSPSLES (SEQ ID NO: 13); a heavy chain CDR3 comprising the amino acid sequence LRSLYTDYDPHYYDY (SEQ ID NO: 14); a light chain CDR1 comprising the amino acid sequence QGNLLPRHYAH (SEQ ID NO: 16); a light chain CDR2 comprising the amino acid sequence DDNIRPS (SEQ ID NO: 17); and a light chain CDR3 comprising the amino acid sequence QSADSNDDAV (SEQ ID NO: 11).

In some embodiments, the isolated antibody comprises a heavy chain variable region amino acid sequence that is identical to SEQ ID NO: 34; and a light chain variable region amino acid sequence that is identical to SEQ ID NO: 35.

In some embodiments, the isolated antibody comprises a heavy chain variable region amino acid sequence that is identical to SEQ ID NO: 36; and a light chain variable region amino acid sequence that is identical to SEQ ID NO: 37.

In some embodiments, the light chain and heavy chain comprise a signal peptide. In some embodiments, the signal peptide comprises an amino acid sequence sequence having at least 70%, at least 75%, at least 80%, at least 85%, at least 90%, at least 91%, at least 92%, at least 93%, at least 94%, at least 95%, at least 96%, at least 97%, at least 98%, or at least 99% homology or identity to the amino acid sequence

(SEQ ID NO: 21)
MGWSCIILFLVATATGVHS,
(SEQ ID NO: 38)
MAWTPLLLPLLTFCTVSEA
or
(SEQ ID NO: 39)
MKHLWFFLLLVAAPRWVLS.

### Engineered Fusion Proteins

In some embodiments, the disclosure provides fusion proteins comprising (i) one or more antigen-binding regions described herein (e.g., antigen-binding region of immunoglobulin, heavy chain antibody, light chain antibody, LRR-based antibody, or other protein scaffold with antibody-like properties, as well as other antigen binding moiety known in the art, including, e.g., a Fab, Fab′, Fab′2, Fab₂, Fab₃, F(ab′)₂, Fd, Fv, Feb, scFv, SMIP, antibody, diabody, triabody, tetrabody, minibody, maxibody, tandab, DVD, BiTe, TandAb, or the like), e.g., one or more variable domains described herein, or portion thereof (e.g., one or more CDRs described herein), and (ii) one or more additional polypeptides. For example, albumin is an abundant serum protein that is protected from degradation by pH-dependent recycling mediated by interaction with FcRn. In some embodiments, one or more variable domains or engineered antibodies as described herein, or portion thereof (e.g., one or more CDRs described herein) is fused to albumin, a portion thereof (such as a portion of albumin that binds to an FcRn), and/or an engineered variant of albumin that binds to FcRn with improved affinity. In other instances, one or more variable domains or engineered antibodies as described herein, or portion thereof (e.g., one or more CDRs described herein) is fused to a polypeptide that binds to albumin to form a fusion protein-albumin complex, which can in turn bind to an FcRn. In some embodiments, the polypeptide that binds to albumin is a single chain variable fragment (scFv). The albumin or portion thereof can include a mutation of one or more amino acids that can modify its binding to an FcRn. Such mutations are known in the art (see, e.g., Andersen et al., Nature Communications 3:610 doi: 10.1038/nocmms1607 (2012)).

In other instances, one or more variable domains or engineered antibodies described herein, or portion thereof (e.g., one or more CDRS described herein) is fused to transferrin. Transferrin is recycled by binding to a transferrin receptor (see, e.g., Widera et al., Adv. Drug Deliv. Rev. 55:1439-66 (2003)).

In some embodiments, the disclosure provides fusion proteins comprising one or more variable domains or engineered antibodies as described herein, or portion thereof, and one or more additional polypeptides and/or scFvs that bind to FcRn.

### Nucleotide Sequences

The present disclosure includes nucleotide sequences encoding one or more heavy chains, heavy chain variable domains, heavy chain framework regions, heavy chain CDRs, heavy chain constant domains, light chains, light chain variable domains, light chain framework regions, light chain CDRs, light chain constant domains, or other immunoglobulin-like sequences, antibodies, or binding molecules disclosed herein. In various instances, such nucleotide sequences may be present in a vector. In various instances such nucleotides may be present in the genome of a cell, e.g., a cell of a subject in need of treatment or a cell for production of an antibody, e.g. a mammalian cell for production of an antibody.

### PEGylation

In certain embodiments, an engineered antibody as described herein can be PEGylated to include mono- or poly-(e.g., 2-4) PEG moieties. Such PEGylated antibodies may display increased half-life in comparison to a non-PEGylated reference antibody, e.g., an antibody having the same amino acid sequence but different, a different amount of, or no PEGylation.

PEGylation can be carried out by any suitable reaction known in the art. Methods for preparing a PEGylated protein can generally include (a) reacting a polypeptide with polyethylene glycol (such as a reactive ester or aldehyde derivative of PEG) under conditions whereby the polypeptide becomes attached to one or more PEG groups; and (b) obtaining the reaction product(s). In general, the conditions for the reactions can be determined case by case based on known parameters and the desired result.

There are a number of PEG attachment methods available to those skilled in the art. For example, the step of PEGylating a multi-specific binding molecule described herein can be carried out via an acylation reaction or an alkylation reaction with a reactive polyethylene glycol molecule.

pH-Dependent Binding of Engineered Antibody with CFD and/or Fc Receptor

Engineered antibodies described herein exhibit pH-dependency, or enhanced pH dependency, in affinity for CFD (e.g., mediated by one or more variable domains described herein), and/or altered (e.g., increased, e.g., pH dependent) affinity for FcRn (e.g., mediated by one or more constant domains described herein). For example, in some embodiments an antibody capable of binding CFD, or a variable domain capable of binding CFD, binds CFD with higher affinity at a serum pH (e.g., at a neutral pH or at a pH above 7.4) than at a compartmental (e.g., endosomal) pH (e.g., at an acidic pH or at a pH equal to or less than pH 6.0). In various instances in which CFD is bound by an antibody having pH-dependent CFD binding, a transition of pH from serum pH to compartmental pH (e.g., from serum to endosome) facilitates separation of CFD and antibody (i.e., “unbinding”) at compartmental pH and/or in a particular compartment, e.g., endosome. In various instances, such pH-dependent binding can mediate antibody recycling and/or CFD degradation. In particular instances, a transition from serum pH to compartmental pH (e.g., from serum to endosome) facilitates separation of CFD and antibody (i.e., “unbinding”) at the compartmental pH and/or in a particular compartment, e.g., endosome, such that the antibody is recycled out by FcRn and the antigen is degraded in a lysosome. In some such instances, the pH-dependency of CFD binding improves the “processivity” of the antibody at least in that, upon recycling, the antibody is returned to serum and is free to bind target circulating CFD. In some instances, recycling of an antibody that displays pH-dependent CFD binding can continue until the antibody eventually degrades or is degraded, by which time a single antibody molecule may have bound and mediated the inactivation of a plurality of CFD molecules, rather than just one.

In certain embodiments, an engineered antibody disclosed herein includes a constant domain (e.g., an Fc domain) displaying increased affinity relative to control (e.g., a reference antibody comprising a heavy chain having the amino acid sequence of SEQ ID NO: 1 and a light chain having the amino acid sequence of SEQ ID NO: 2), for an Fc receptor, such as FcRn. In some embodiments, such increased affinity relative to control is at a pH value for serum (e.g., pH greater than 6.8, 6.9, 7.0, 7.1, 7.2, 7.3, 7.4, 7.5, 7.6, 7.7, 7.8, 7.9, 8.0, 8.1, 8.2, or greater). In some embodiments, such increased affinity relative to control is at a compartmental pH (e.g., a pH lower than 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, or lower). In certain embodiments, an engineered antibody disclosed herein includes a constant domain (e.g., an Fc domain) displaying pH-dependency (or enhanced pH dependency relative to control, e.g., a reference antibody comprising a heavy chain having the amino acid sequence of SEQ ID NO: 1 and a light chain having the amino acid sequence of SEQ ID NO: 2), in affinity for an Fc receptor, such as FcRn. The neonatal Fc receptor (FcRn) is a MHC class I like molecule that functions to protect IgG and albumin from catabolism, mediates transport of IgG across epithelial cells, and is involved in antigen presentation by professional antigen presenting cells. IgG antibody subtypes exhibit long serum half-lives, primarily due to the scavenging of antibodies from the endosomes by FcRn that recycles IgGs back out of cells.

In some particular examples, an engineered antibody as described herein displays greater pH-dependency in binding with FcRn than does endogenous IgG, in that antibodies as described herein display a greater absolute and/or relative differential change in affinity between serum pH and compartmental (e.g., endosomal) pH (or between serum and endosome) than does endogenous IgG. In some instances, an antibody having pH-dependent binding with FcRn displays greater binding to FcRn than do endogenous IgGs at a compartmental (e.g., endosomal) pH (e.g., at an acidic pH or at a pH equal to or less than pH 6.0). In some instances, an antibody having pH-dependent binding with FcRn displays greater binding to FcRn than do endogenous IgGs at a serum pH (e.g., in serum, at a neutral pH, or at a pH above pH 7.4).

In certain instances, an engineered antibody described herein displays pH-dependency, or enhanced pH dependency, in binding with FcRn and competes with endogenous IgG for interaction with FcRn. Accordingly, in some instances, an engineered antibody described herein binds FcRn at a level that is greater than endogenous IgG molecules (i.e., out-competes endogenous IgG molecules for binding with FcRn as a result of its greater affinity for FcRn) and/or results in a net increase in the rate of recycling of the FcRn-affinity-enhanced antibody relative to endogenous IgG molecules. In some instances, such preferential interaction of an engineered antibody described herein with FcRn, relative to endogenous IgG molecules, results in increased antibody half-life as compared to a reference antibody.

In some embodiments, an engineered antibody described herein exhibits a) increased affinity for FcRn at an acidic pH or compartmental pH, relative to that at serum pH and/or b) decreased affinity for CFD at an acidic pH or compartmental pH relative to that at serum pH. In some instances, such combination of features results in a synergistic increase in the processivity of the antibody that facilitates degradation of antigen, reduces buildup of antigen-antibody complex, and/or in cases where the antigen-antibody complex is cleared faster than the antibody itself it significantly increases half-life of antibody. This approach allows highly effective targeting and clearance of CFD.

In some embodiments, where an engineered antibody has a higher affinity for CFD at serum pH than at compartmental pH (e.g., endosomal pH) and further includes an FcRn binding moiety that has a lower affinity for FcRn at serum pH than at compartmental pH (e.g., endosomal pH), such antibody binds serum CFD with high affinity to form an antibody/CFD complex. Upon internalization of the antibody/CFD complex by a cell (e.g., by pinocytosis) and into an internal compartment (e.g., an endosome) having a lower pH than serum pH, the lower affinity of antibody for CFD facilitates separation of antibody and CFD, whereafter released CFD can be degraded by cellular machinery (e.g., by lysosomes). Further, increased affinity for FcRn at compartmental pH facilitates formation of antibody/FcRn complex, which complex can be recycled back into serum via the FcRn recycling pathway, within which the complex is exposed to serum or serum-like pH conditions, which pH facilitates release of antibody from the FcRn complex, e.g., back into serum. One net result of such processes can be a reduction the serum half-life of CFD. Another net result of such processes can be an increase in serum half-life of antibody.

In various instances as disclosed herein, a serum pH can be, e.g., a pH or pH range typical or characteristic of serum, an individual or mean serum pH or pH range for one or more subjects, a standard pH value for serum, a measured pH value for serum, an estimated pH value for serum, or a selected pH value for serum. In various instances as disclosed herein, a serum pH can be a pH greater than 6.8, 6.9, 7.0, 7.1, 7.2, 7.3, 7.4, 7.5, 7.6, 7.7, 7.8, 7.9, 8.0, 8.1, 8.2, or greater. In various instances, a serum pH is a pH in the range of 6.8 to 8.2, 7.0 to 8.0, 7.0 to 7.8, 7.0 to 7.6, 7.0 to 7.4, or 7.0 to 7.2. In some embodiments, a serum pH is at or about pH 7.4

In various instances as disclosed herein, a compartmental pH can be, e.g., a pH or pH range typical or characteristic of an endosomal compartment (e.g., within an endosome), an individual or mean endosomal compartment pH or pH range for one or more subjects, a standard pH value for an endosomal compartment, a measured pH value for an endosomal compartment, an estimated pH value for an endosomal compartment, or a selected pH value for an endosomal compartment. In various instances as disclosed herein, a compartmental pH can be a pH lower than 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, or lower. In various instances, a compartmental pH is a pH in the range of 5.0 to 7.2, 5.0 to 7.0, 5.0 to 6.8, 5.0 to 6.6, 5.0 to 6.4, 5.0 to 6.2, 5.0 to 6.0, 5.0 to 5.8, 5.0 to 5.6, 5.0 to 5.4, or 5.0 to 5.2.

In some embodiments, serum half-life of a serum CFD protein is decreased. For example, binding of an engineered antibody to serum CFD reduces serum half-life of CFD to fewer than about 7, 6, 5, 4, 3, 2, or 1 days, or less than about 24, 18, 12, or 6 hours.

In some embodiments, serum half-life of an engineered antibody is increased. For example, binding of an engineered antibody to FcRn increases serum half-life of the antibody to about 4 days to about 45 days, e.g., about 5 days to about 30 days, about 10 days to about 30 days, or about 20 days to about 30 days. In certain embodiments, an engineered antibody described herein has a serum half-life of about 5 days, about 10 days, about 15 days, about 20 days, about 25 days, about 30 days, about 35 days, about 40 days, about 45 days, about 50 days or longer.

In certain embodiments, an engineered antibody described herein exhibits a pH-dependent change in affinity for CFD. Affinity may be measured as a K_{D}, equilibrium dissociation constant, of antibody and antigen; K_{D} and affinity are inversely related. In various embodiments, K_{D} of an engineered antibody as described herein for CFD at a serum pH (e.g., a pH greater than 6.8, 6.9, 7.0, 7.1, 7.2, 7.3, 7.4, 7.5, 7.6, 7.7, 7.8, 7.9, 8.0, 8.1, 8.2, or greater) or under serum conditions is less than about 10⁻⁴, 10⁻⁵, 10⁻⁶, 10⁻⁷, 10⁻⁸, 10⁻⁹, 10⁻¹⁰, 10⁻¹¹, 10⁻¹², 10⁻¹³, 10⁻¹⁴, or 10⁻¹⁵ M. In certain instances, K_{D} of an antibody as described herein for CFD at a serum pH is between 0.001 and 1 nM, e.g., 0.001 nM, 0.005 nM, 0.01 nM, 0.05 nM, 0.1 nM, 0.5 nM, or 1 nM. In some embodiments, K_{D} for CFD at a compartmental pH (e.g., a pH lower than 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, or lower) or under compartmental conditions is higher than K_{D} of the same antibody for CFD at a serum pH or under serum conditions (and/or affinity of antibody for CFD at compartmental pH or under compartmental conditions may be decreased relative to affinity at a serum pH or under serum conditions) by, e.g., at least 2-fold, e.g., 2-fold, 3-fold, 4-fold, 5-fold, 6-fold, 7-fold, 8-fold, 9-fold, 10-fold, 20-fold, 30-fold, 40-fold, 50-fold, 75-fold, 100-fold 150-fold, 200-fold, 250-fold, 300-fold, 350-fold, 400-fold, 450-fold, 500-fold, 600-fold, 700-fold, 800-fold, 900-fold, 1000-fold, 2,000-fold, 3,000-fold, 4,000-fold, 5,000-fold, 6,000-fold, 7-000 fold, 8,000-fold, 9,000-fold, 10,000-fold, or more. In some embodiments, K_{D} for CFD at a compartmental pH (e.g., a pH lower than 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, or lower) or under compartmental conditions may be, e.g., greater than 10⁻¹⁵, 10⁻¹⁴, 10⁻¹³, 10⁻¹², 10⁻¹¹, 10⁻¹⁰, 10⁻⁹, 10⁻⁸, 10⁻⁷, 10⁻⁶, 10⁻⁵, 10⁻⁴, or 10⁻³ M. In certain instances, K_{D} of an engineered antibody as described herein for CFD at a compartmental pH or under compartmental conditions may be, e.g., equal to or greater than 1 nM, e.g., 1 nM, 2 nM, 3 nM, 4 nM, 5 nM, 10 nM, 20 nM, 30 nM, 40 nM, 50 nM, 100 nM, 200 nM, 300 nM, 400 nM, 500 nM, 600 nM, 700 nM, 800 nM, 900 nM, 1 mM, or more.

In some embodiments, an engineered antibody described herein exhibits a pH-dependent change in affinity for a receptor, such as an Fc receptor, e.g., FcRn. In various embodiments, K_{D} of an engineered antibody as described herein for FcRn at a compartmental pH (e.g., a pH lower than 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, or lower) or under compartmental conditions may be less than 10⁻⁴, 10⁻⁵, 10⁻⁶, 10⁻⁷, 10⁻⁸, 10⁻⁹, 10⁻¹⁰, 10⁻¹¹, 10⁻¹², 10⁻¹³, 10⁻¹⁴, or 10⁻¹⁵ M. In some embodiments, K_{D} for FcRn at a serum pH (e.g., a pH greater than 6.8, 6.9, 7.0, 7.1, 7.2, 7.3, 7.4, 7.5, 7.6, 7.7, 7.8, 7.9, 8.0, 8.1, 8.2, or greater) or under serum conditions is higher than K_{D} of the same antibody for FcRn at a compartmental pH or under compartmental conditions (and/or affinity of antibody for FcRn at serum pH or under serum conditions may be decreased relative to affinity at a compartmental pH or under compartmental conditions), e.g., higher by at least 2-fold, e.g., 2-fold, 3-fold, 4-fold, 5-fold, 6-fold, 7-fold, 8-fold, 9-fold, 10-fold, 20-fold, 30-fold, 40-fold, 50-fold, 75-fold, 100-fold 150-fold, 200-fold, 250-fold, 300-fold, 350-fold, 400-fold, 450-fold, 500-fold, 600-fold, 700-fold, 800-fold, 900-fold, 1000-fold, 2,000-fold, 3,000-fold, 4,000-fold, 5,000-fold, 6,000-fold, 7-000 fold, 8,000-fold, 9,000-fold, 10,000-fold, or more. In some embodiments, K_{D} for FcRn at a serum pH (e.g., a pH greater than 6.8, 6.9, 7.0, 7.1, 7.2, 7.3, 7.4, 7.5, 7.6, 7.7, 7.8, 7.9, 8.0, 8.1, 8.2, or greater) or under serum conditions is, e.g., greater than 10⁻¹⁵, 10⁻¹⁴, 10⁻¹³, 10⁻¹², 10⁻¹¹, 10⁻¹⁰, 10⁻⁹, 10⁻⁸, 10⁻⁷, 10⁻⁶, 10⁻⁵, 10⁻⁴, or 10⁻³ M. In certain instances, K_{D} of an engineered antibody as described herein for FcRn at a serum pH or under serum conditions is, e.g., equal to or greater than 1 nM, e.g., 1 nM, 2 nM, 3 nM, 4 nM, 5 nM, 10 nM, 20 nM, 30 nM, 40 nM, 50 nM, 100 nM, 200 nM, 300 nM, 400 nM, 500 nM, 600 nM, 700 nM, 800 nM, 900 nM, 1 mM, or more.

In some embodiments, an engineered antibody described herein exhibits both a pH-dependent change in affinity for CFD and a pH-dependent change an affinity for Fc receptor, e.g., FcRn. Thus, in some embodiments, an engineered antibody described herein exhibits greater affinity for CFD under serum conditions or at serum pH than under compartment conditions or at compartmental pH and also exhibits greater affinity for FcRn under compartmental conditions or at compartmental pH than under serum conditions or at serum pH. In various embodiments, K_{D} of an engineered antibody for CFD at a serum pH (e.g., a pH greater than 6.8, 6.9, 7.0, 7.1, 7.2, 7.3, 7.4, 7.5, 7.6, 7.7, 7.8, 7.9, 8.0, 8.1, 8.2, or greater) or under serum conditions may be less than 10⁻⁴, 10⁻⁵, 10⁻⁶, 10⁻⁷, 10⁻⁸, 10⁻⁹, 10⁻¹⁰, 10⁻¹¹, 10⁻¹², 10⁻¹³, 10⁻¹⁴, or 10⁻¹⁵ M. In certain instances, K_{D} of an engineered antibody for CFD at a serum pH is between 0.001 and 1 nM, e.g., 0.001 nM, 0.005 nM, 0.01 nM, 0.05 nM, 0.1 nM, 0.5 nM, or 1 nM. In certain instances, K_{D} of an engineered antibody as described herein for CFD at a compartmental pH is between 0.001 and 1 nM, e.g., 0.001 nM, 0.005 nM, 0.01 nM, 0.05 nM, 0.1 nM, 0.5 nM, or 1 nM. In some embodiments, K_{D} for CFD at a compartmental pH (e.g., a pH lower than 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, or lower) or under compartmental conditions is higher than K_{D} of the same antibody for CFD at a serum pH or under serum conditions (and/or affinity of antibody for CFD at compartmental pH or under compartmental conditions is decreased relative to affinity at a serum pH or under serum conditions) by, e.g., at least 2-fold, e.g., 2-fold, 3-fold, 4-fold, 5-fold, 6-fold, 7-fold, 8-fold, 9-fold, 10-fold, 20-fold, 30-fold, 40-fold, 50-fold, 75-fold, 100-fold 150-fold, 200-fold, 250-fold, 300-fold, 350-fold, 400-fold, 450-fold, 500-fold, 600-fold, 700-fold, 800-fold, 900-fold, 1000-fold, 2,000-fold, 3,000-fold, 4,000-fold, 5,000-fold, 6,000-fold, 7-000 fold, 8,000-fold, 9,000-fold, 10,000-fold, or more. In some embodiments, K_{D} for CFD at a compartmental pH (e.g., a pH lower than 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, or lower) or under compartmental conditions is, e.g., greater than 10⁻¹⁵, 10⁻¹⁴, 10⁻¹³, 10⁻¹², 10⁻¹¹, 10⁻¹⁰, 10⁻⁹, 10⁻⁸, 10⁻⁷, 10⁻⁶, 10⁻⁵, 10⁻⁴, or 10⁻³ M. In certain instances, K_{D} of an engineered antibody for CFD at a compartmental pH or under compartmental conditions is, e.g., equal to or greater than 1 nM, e.g., 1 nM, 2 nM, 3 nM, 4 nM, 5 nM, 10 nM, 20 nM, 30 nM, 40 nM, 50 nM, 100 nM, 200 nM, 300 nM, 400 nM, 500 nM, 600 nM, 700 nM, 800 nM, 900 nM, 1 mM, or more. In some embodiments, K_{D} of an engineered antibody for FcRn at a serum pH (e.g., a pH greater than 6.8, 6.9, 7.0, 7.1, 7.2, 7.3, 7.4, 7.5, 7.6, 7.7, 7.8, 7.9, 8.0, 8.1, 8.2, or greater) or under serum conditions is higher than K_{D} of the same antibody for FcRn at a compartmental pH or under compartmental conditions (and/or affinity of antibody for FcRn at serum pH or under serum conditions is decreased relative to affinity at a compartmental pH or under compartmental conditions) by, e.g., at least 2-fold, e.g., 2-fold, 3-fold, 4-fold, 5-fold, 6-fold, 7-fold, 8-fold, 9-fold, 10-fold, 20-fold, 30-fold, 40-fold, 50-fold, 75-fold, 100-fold 150-fold, 200-fold, 250-fold, 300-fold, 350-fold, 400-fold, 450-fold, 500-fold, 600-fold, 700-fold, 800-fold, 900-fold, 1000-fold, 2,000-fold, 3,000-fold, 4,000-fold, 5,000-fold, 6,000-fold, 7-000 fold, 8,000-fold, 9,000-fold, 10,000-fold, or more. In some embodiments, K_{D} of an engineered antibody for FcRn at a serum pH (e.g., a pH greater than 6.8, 6.9, 7.0, 7.1, 7.2, 7.3, 7.4, 7.5, 7.6, 7.7, 7.8, 7.9, 8.0, 8.1, 8.2, or greater) or under serum conditions is, e.g., greater than 10⁻¹⁵, 10⁻¹⁴, 10⁻¹³, 10⁻¹², 10⁻¹¹, 10⁻¹⁰, 10⁻⁹, 10⁻⁸, 10⁻⁷, 10⁻⁶, 10⁻⁵, 10⁻⁴, or 10⁻³ M. In certain instances, K_{D} of an engineered antibody for FcRn at a serum pH or under serum conditions is, e.g., equal to or greater than 1 nM, e.g., 1 nM, 2 nM, 3 nM, 4 nM, 5 nM, 10 nM, 20 nM, 30 nM, 40 nM, 50 nM, 100 nM, 200 nM, 300 nM, 400 nM, 500 nM, 600 nM, 700 nM, 800 nM, 900 nM, 1 mM, or more. In some embodiments, K_{D} of an engineered antibody for FcRn at a compartmental pH (e.g., a pH lower than 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, or lower) or under compartmental conditions is less than 10⁻⁴, 10⁻⁵, 10⁻⁶, 10⁻⁷, 10⁻⁸, 10⁻⁹, 10⁻¹⁰, 10⁻¹¹, 10⁻¹², 10⁻¹³, 10⁻¹⁴, or 10⁻¹⁵ M.

In some embodiments, an engineered antibody described herein exhibits a greater half-life than a reference antibody (e.g., a reference antibody comprising a heavy chain having the amino acid sequence of SEQ ID NO: 1 and a light chain having the amino acid sequence of SEQ ID NO: 2) when administered to a subject, e.g., in the serum of the subject. In various instances, the half-life of a reference antibody (e.g., a reference antibody comprising a heavy chain having the amino acid sequence of SEQ ID NO: 1 and a light chain having the amino acid sequence of SEQ ID NO: 2) in serum may be, e.g., 250 to 300 hours. In various instances, the half-life in serum of an engineered antibody as described herein may be, e.g., at least 250 hours, e.g., at least 260, 270, 280, 290, or 300 hours. In certain embodiments, the half-life in serum of an engineered antibody as described herein may be at least 300 hours, e.g., at least 350, 400, 450, 500, 550, 600, 650, 700, 750, 800, 850, 900, 950, or 1,000 hours. In certain embodiments, the half-life in serum of an engineered antibody as described herein may be at least 1,000 hours, e.g., at least 1,500, 2,000, 2,500, 3,000, 3,500, 4,000, 4,500, 5,000, 6,000, 7,000, 8,000, 9,000, 10,000, 11,000, 12,000, 13,000, 14,000, or 15,000 hours or more. In various embodiments, the half-life in serum of an engineered antibody as described herein may be at least 12 days, 15 days, 20 days, 25 days, 30 days, 35 days, 40 days, 45 days, 50 days, 2 months, 3 months, 4 months, 5 months, 6 months, or more. In various instances, the half-life in serum of an engineered antibody as described herein may be increased as compared to a reference antibody (e.g., a reference antibody comprising a heavy chain having the amino acid sequence of SEQ ID NO: 1 and a light chain having the amino acid sequence of SEQ ID NO: 2) by a factor of at least, e.g., 2-fold, 3-fold, 4-fold, 5-fold, 6-fold, 7-fold, 8-fold, 9-fold, 10-fold, 20-fold, 30-fold, 40-fold, 50-fold, 75-fold, 100-fold or more.

In certain embodiments, an engineered antibody described herein exhibits an increased half-life in plasma, an increased mean retention time in plasma, and/or an increased level of CFD clearance (e.g., relative to a reference antibody, e.g., a reference antibody comprising a heavy chain having the amino acid sequence of SEQ ID NO: 1 and a light chain having the amino acid sequence of SEQ ID NO: 2). These parameters can be determined by methods known to those skilled in the art (e.g., as described in Nestorov et al., J. Clin. Pharmacol. 48:406-417 (2008); Leveque et al., Anticancer Research 25:2327-2344 (2005); Igawa et al., PLoS One 8: e63236. doi: 10.1371/journal.pone.0063236 (2013)). For example, an engineered antibody described herein (e.g., a single dose of such engineered antibody) reduces level of CFD in plasma by at least 10-fold, 50-fold, 100-fold, 250-fold, 500-fold, 750-fold, 1000-fold, 1500-fold, or more, relative to a reference antibody, e.g., a reference antibody comprising a heavy chain having the amino acid sequence of SEQ ID NO: 1 and a light chain having the amino acid sequence of SEQ ID NO: 2.

In some embodiments, an engineered antibody described herein inhibits cleavage of CFD (and/or levels of CFD in serum). In various embodiments, an engineered antibody as disclosed herein inhibits cleavage of CFD (and/or levels of CFD in serum) as compared to a prior measurement from the same patient or a reference value. In particular embodiments, administration of an engineered antibody as disclosed herein decreases the level or amount of CFD cleavage (and/or level or amount of CFDa and/or level or amount of CFDb) more than comparable administration of a reference antibody, e.g., a reference antibody comprising a heavy chain having the amino acid sequence of SEQ ID NO: 1 and a light chain having the amino acid sequence of SEQ ID NO: 2 (e.g., decreases level by more than about 10%, 20%, 30%, 40%, 50%, 60%, 70%, 80%, 90%, 100%, 150%, 2005, or more).

### Engineered Multi-Specific Molecules

Methods and compositions described herein in the context of engineered anti-CFD antibodies can be applied to additional proteins to produce multi-specific binding molecules. Multi-specific binding molecules according to the present disclosure are engineered to include one or more binding moieties that specifically bind one or more targets of interest in a pH-dependent manner. Multi-specific binding molecules encompass nucleic acids (e.g., RNA and DNA), proteins (e.g., antibodies), and combination thereof pH-dependent binding moieties can be or include, for example, nucleic acids (e.g., RNA and DNA) and aptamers, polypeptides (e.g., antibodies or fragments thereof, albumin, receptors, ligands, signal peptides, avidin, and Protein A), polysaccharides, biotin, hydrophobic groups, hydrophilic groups, drugs, and any organic molecules that bind to receptors.

### Antibody or Fragment Thereof as Binding Moieties

In some embodiments, a multi-specific binding molecule described herein is an engineered antibody. In some instances, one or more binding moieties described herein are or include antibodies, antigen-binding fragments thereof, and/or Fc regions (or Fc fragments) thereof. The basic structure of an IgG antibody consists of two identical light polypeptide chains and two identical heavy polypeptide chains linked together by disulphide bonds. The first domain located at the amino terminus of each chain is variable in amino acid sequence, providing antibody binding specificities found in each individual antibody. These are known as variable heavy (VH) and variable light (VL) regions. The other domains of each chain are relatively invariant in amino acid sequence and are known as constant heavy (CH) and constant light (CL) regions. For an IgG antibody, the light chain includes one variable region (VL) and one constant region (CL). An IgG heavy chain includes a variable region (VH), a first constant region (CH1), a hinge region, a second constant region (CH2), and a third constant region (CH3). In IgE and IgM antibodies, the heavy chain includes an additional constant region (CH4).

Antibodies described herein can include, for example, monoclonal antibodies, polyclonal antibodies, multispecific antibodies, human antibodies, humanized antibodies, camelized antibodies, chimeric antibodies, single-chain Fvs (scFv), disulfide-linked Fvs (sdFv), and anti-idiotypic (anti-Id) antibodies, and antigen-binding fragments of any of the above. Antibodies can be of any type (e.g., IgG, IgE, IgM, IgD, IgA and IgY), class (e.g., IgG1, IgG2, IgG3, IgG4, IgA1 and IgA2) or subclass.

The term “Fc fragment”, as used herein, refers to one or more fragments of an Fc region that retains an Fc function and/or activity described herein, such as binding to an Fc receptor. The term “antigen binding fragment” of an antibody, as used herein, refers to one or more fragments of an antibody that retain the ability to specifically bind to an antigen. Examples of binding fragments encompassed within the term “antigen binding fragment” of an antibody include a Fab fragment, a F(ab′)₂ fragment, a Fd fragment, a Fv fragment, a scFv fragment, a dAb fragment (Ward et al., (1989) Nature 341:544-546), and an isolated complementarity determining region (CDR). These antibody fragments can be obtained using conventional techniques known to those with skill in the art, and fragments can be screened for utility in the same manner as are intact antibodies.

Antibodies or fragments can be produced by any method known in the art for synthesizing antibodies (see, e.g., Harlow et al., Antibodies: A Laboratory Manual, (Cold Spring Harbor Laboratory Press, 2nd ed. 1988); Brinkman et al., 1995, J. Immunol. Methods 182:41-50; WO 92/22324; WO 98/46645). Chimeric antibodies can be produced using methods described in, e.g., Morrison, 1985, Science 229:1202, and humanized antibodies by methods described in, e.g., U.S. Pat. No. 6,180,370.

Additional antibodies of compositions and methods described herein are bispecific antibodies and multivalent antibodies, as described in, e.g., Segal et al., J. Immunol. Methods 248:1-6 (2001); and Tutt et al., J. Immunol. 147: 60 (1991).

In some embodiments, a multi-specific molecule described herein is an engineered antibody (e.g., engineered to have pH sensitive binding to antigen and to FcRn).

Engineered Antigen Binding Regions of Engineered Multi-Specific Molecules

In some embodiments, a binding moiety is or includes an antibody (e.g., an IgG antibody, e.g., an IgG1, IgG2, or IgG3 antibody), or an antigen binding fragment, engineered to bind to a target (i.e., antigen) in an altered manner (e.g., in a pH sensitive manner, e.g., in a more or less pH sensitive manner) relative to a reference antibody or antigen binding fragment. For example, an antibody can be engineered by modifying (e.g., by adding, deleting, or substituting) an amino acid within one or more antibody CDRs and/or at a position involved in antibody CDR structure. Exemplary, non-limiting sites of an antibody that can be modified include the following (amino acid positions are indicated based on the Kabat numbering (Kabat et al., (1991) Sequences of Proteins of Immunological Interest, NIH)).

Heavy chain: H27, H31, H32, H33, H35, H50, H58, H59, H61, H62, H63, H64, H65, H99, H100b, and H102

Light chain: L24, L27, L28, L32, L53, L54, L56, L90, L92, and L94.

In some embodiments, one or more of these disclosed amino acids can be substituted with histidine, arginine, lysine, aspartic acid, glutamic acid, serine, threonine, asparagine, or glutamine. Without wishing to be bound by theory, it is believed that substituting an amino acid at one or more of these positions with a histidine can result in an antibody having pH-dependent antigen-binding properties. In some embodiments, a non-histidine residue is substituted with a histidine residue. In some embodiments, a histidine residue is substituted with a non-histidine residue. Additional engineered antigen binding regions include those described in, e.g., U.S. Publ. No. 20110229489.

Engineered Constant Regions ofEngineeredMulti-Specific Molecules

In some instances, a binding moiety is or includes an antibody constant region, Fc region or Fc fragment that binds one or more Fc receptors (e.g., FcγRI, FcγRIIA, FcγRIIB, FcγRIIIA, FcγRIIIB, FcγRIV, or FcRn receptor). In some embodiments, a constant region, Fc region or Fc fragment is engineered to bind to a target (e.g., an Fc receptor) in an altered manner (e.g., in a pH sensitive manner, e.g., in a more or less pH sensitive manner) relative to a reference constant region, Fc region or Fc fragment.

In some instances, a binding moiety can be or include a constant region, Fc region or Fc fragment of an IgG antibody engineered to include an amino acid addition, deletion, or substitution, of one or more of amino acid residues described herein (e.g., 251-256, 285-290, 308-314, 385-389, and 428-436 (Kabat numbering (Kabat et al., (1991) Sequences of Proteins of Immunological Interest, NIH))).

Producing Multi-Specific Binding Molecules

In some embodiments, a multi-specific binding molecule described herein is engineered to include one or more binding moieties that exhibit pH sensitive binding to one or more targets by mutagenesis using known techniques. For example, a sequence of a reference polypeptide (e.g., a therapeutic antibody or therapeutic fusion protein) can be obtained, and one or more amino acid residues can be added, deleted, or substituted. In some embodiments, one or more amino acid residues are substituted with histidine, arginine, lysine, aspartic acid, glutamic acid, serine, threonine, asparagine, or glutamine. In some embodiments, one or more amino acids are substituted with histidine. Without wishing to be bound by theory, it is believed that substitution of an amino acid residue with a histidine results in insertion of a protonation site, which can increase pH sensitivity of a binding moiety. Polypeptides can be produced using standard methods and assayed for binding to targets of interest as described herein. Additional methods of increasing pH sensitivity of a binding moiety are described in, e.g., Sarkar et al., Nature Biotechnology 20:908-913 (2002); Murtaugh et al., Protein Science 20:1619-1631 (2011); and U.S. Publ. No. 20110229489.

In some embodiments, a first target of interest is selected, and an antibody that selectively binds to the target is provided, obtained, and/or produced (e.g., using known methods as described herein). One or more amino acids of an antigen-binding region and/or an Fc region are substituted (e.g., with histidine, arginine, lysine, aspartic acid, glutamic acid, serine, threonine, asparagine, or glutamine), and pH sensitivity of binding to the target (and, additionally or alternatively, to FcRn) is determined. An antibody demonstrating desired binding affinity is selected as a multi-specific binding molecule.

In some embodiments, a polypeptide that naturally binds to a target of interest is provided, obtained, and/or produced. The polypeptide is conjugated to an Fc region or Fc fragment described herein (e.g., which binds to FcRn with a desired binding affinity) using known methods. For example, the polypeptide and Fc region or Fc fragment can be conjugated by chemical means or by recombinant expression as a fusion protein. Additionally or alternatively, one or more amino acids of the polypeptide can be substituted (e.g., with histidine, arginine, lysine, aspartic acid, glutamic acid, serine, threonine, asparagine, or glutamine), and pH sensitivity of binding of the polypeptide and the target is determined.

In some embodiments, a multi-specific binding molecule described herein is engineered to include one or more binding moieties identified and/or selected by screening. For example, an antigen-binding moiety that binds antigen in a pH sensitive manner can be identified using a library, e.g., a phage library, expressing antigen-binding moieties. Such a library can be screened for antigen-binding moieties that have a first affinity for antigen at a first pH (e.g., at pH 7.4) and that have a second affinity for antigen at a second pH (e.g., at pH 5.5). A multi-specific binding molecule described herein can be engineered to include such identified pH-sensitive antigen-binding moieties. Additionally and/or alternatively, an FcRn-binding moiety that binds FcRn in a pH sensitive manner can be identified using a library. Methods of screening recombinant antibody libraries are known (see, e.g., Hoogenboom, Nature Biotech. 23:1105-1116 (2005); U.S. Pat. Nos. 5,837,500; 5,571,698; WO 2012/044831).

### Measuring Interactions of Binding Moieties and Targets

The binding properties of a multi-specific binding molecule described herein (e.g., an engineered antibody described herein) to a target (e.g., CFD and/or FcRn) can be measured by methods known in the art, e.g., one of the following methods: BIACORE analysis, Enzyme Linked Immunosorbent Assay (ELISA), x-ray crystallography, sequence analysis and scanning mutagenesis. The binding interaction of an antibody and CFD and/or FcRn can be analyzed using surface plasmon resonance (SPR). SPR or Biomolecular Interaction Analysis (BIA) detects bio-specific interactions in real time, without labeling any of the interactants. Changes in the mass at the binding surface (indicative of a binding event) of the BIA chip result in alterations of the refractive index of light near the surface. The changes in the refractivity generate a detectable signal, which are measured as an indication of real-time reactions between biological molecules. Methods for using SPR are described, for example, in U.S. Pat. No. 5,641,640; Raether (1988) Surface Plasmons Springer Verlag; Sjolander and Urbaniczky (1991) Anal. Chem. 63:2338-2345; Szabo et al. (1995) Curr. Opin. Struct. Biol. 5:699-705 and on-line resources provide by BIAcore International AB (Uppsala, Sweden). Additionally, a KinExA® (Kinetic Exclusion Assay) assay, available from Sapidyne Instruments (Boise, Id.) can also be used.

Information from SPR can be used to provide an accurate and quantitative measure of the equilibrium dissociation constant (K_{D}), and kinetic parameters, including Kₒₙ and K_{off}, for the binding of a binding moiety to a target (e.g., an engineered antibody to CFD and/or FcRn). Such data can be used to compare different molecules. Information from SPR can also be used to develop structure-activity relationships (SAR). For example, the kinetic and equilibrium binding parameters of particular binding moieties to targets at various pH levels can be evaluated. Variant amino acids at given positions can be identified that correlate with particular binding parameters, e.g., high affinity, low affinity, and slow K_{off}, at particular pH levels.

### Methods of Treatment

In some embodiments, a multi-specific binding molecule described herein (e.g., an engineered antibody as described herein) is used in a method of treating one or more complement-associated conditions. In some embodiments, a multi-specific binding molecule described herein (e.g. an engineered antibody as described herein) is for use as a medicament. Complement-associated conditions can include, without limitation, conditions that are caused by, include, include symptoms resulting in whole or in part from, or are known to occur in conjunction with increased or decreased complement activity. CFD is a key to pathology and amenable to CFD blockade in hematologic, neurologic and renal disorders among others. Examples of complement-associated conditions include, without limitation, peripheral neuropathy, cryoglobulinemia, cryoglobulinemic neuropathy, neurosarcoidosis age-related macular degeneration (AMD), Alzheimer's disease, amyotrophic lateral sclerosis (ALS), antiphospholipid syndrome (or antiphospholipid antibody syndrome or Hughes syndrome), vasculitic neuropathy, Reflex Sympathetic Dystrophy, Complex Regional Pain Syndrome, Chronic Inflammatory Demyelinating Polyneuropathyantineutrophil cytoplasm antibody (ANCA)-associated vasculitis (AAV), asthma, atherosclerosis, atypical hemolytic-uremic syndrome (aHUS), autoimmune hemolytic anemia, brain injury, C3 nephropathy, capillary leak syndrome, cardiopulmonary bypass and hemodialysis, cardiovascular disorders, catastrophic antiphospholipid syndrome, cerebrovascular disorders, chronic inflammatory demyelinating neuropathy, cold agglutinin disease (CAD), Degos disease, dense deposit disease (DDD), dermatitis, inflammatory myopathies, dermatomyositis, myositis, antibody-induced myositis, diabetic angiopathy, diabetic retinopathy, dilated cardiomyopathy, Disseminated Intravascular Coagulation (DIC), elevated liver enzymes, epidermolysis bullosa, erythematosus-associated vasculitis, glomerulonephritis, Goodpastures syndrome, Graves' disease, Guillain-Barre syndrome (GBS), Hashimoto's thyroiditis, HELLP syndrome, hemolysis, Sickle Cell Disease Henoch-Schonlein purpura nephritis, idiopathic thrombocytopenic purpura (ITP), injury resulting from myocardial infarction, ischemia-reperfusion injury, Kawasaki's disease, lupus nephritis, immune complex vasculitis, macular degeneration (e.g., age-related macular degeneration (AMD), mesenteric/enteric vascular disorders, multifocal motor neuropathy, multiple sclerosis, myasthenia gravis, myocarditis, Neonatal Allo-Immune Thrombocytopenia (NAITP), neuromyelitis optica, organ or tissue transplantation, paroxysmal cold hemoglobinuria, paroxysmal nocturnal hemoglobinuria (PNH), Pauci-immune vasculitis, pemphigus, percutaneous transluminal coronary angioplasty, peripheral vascular disorders, Post-Transfusion Purpura, psoriasis, recurrent fetal loss, renovascular disorders, restenosis following stent placement, revascularization to transplants and/or replants, rotational atherectomy, rheumatoid arthritis, psoriatic arthritis, scleroderma, sepsis, septic shock, shiga toxin E. coli-related hemolytic uremic syndrome (STEC-HUS), spinal cord injury, spontaneous fetal loss, systemic inflammatory response, glomerulonephritis, systemic lupus, systemic lupus erythematosus (SLE), systemic lupus erythematosus-associated vasculitis, Takayasu's disease, thoracic-abdominal aortic aneurysm, thrombotic thrombocytopenic purpura (TTP), transplant rejection, traumatic brain injury, type I diabetes, typical or infectious hemolytic uremic syndrome, vasculitis, vasculitis associated with rheumatoid arthritis, and venous gas embolus, or any complement-associated inflammatory response.

Complement-associated disorders include complement-associated pulmonary disorders such as, but not limited to, asthma, bronchitis, a chronic obstructive pulmonary disease (COPD), an interstitial lung disease, α1 anti-trypsin deficiency, emphysema, bronchiectasis, bronchiolitis obliterans, alveolitis, sarcoidosis, pulmonary fibrosis, and collagen vascular disorders.

In some embodiments, a multi-specific binding molecule described herein (e.g., an engineered antibody described herein) can be used to treat graft rejection/graft-versus-host disease (GVHD), reperfusion injuries (e.g., following cardiopulmonary bypass or a tissue transplant), and tissue damage following other forms of traumatic injury such as a burn (e.g., a severe burn), blunt trauma, spinal injury, or frostbite. In some embodiments, a multi-specific binding molecule described herein (e.g., an engineered antibody described herein), alone or in combination with a second anti-inflammatory agent, can be used to treat an inflammatory disorder such as, but not limited to, RA (above), inflammatory bowel disease, sepsis (above), septic shock, acute lung injury, disseminated intravascular coagulation (DIC), or Crohn's disease. In some embodiments, the second anti-inflammatory agent can be one selected from the group consisting of NSAIDs, corticosteroids, methotrexate, hydroxychloroquine, anti-TNF agents such as etanercept and infliximab, a B cell depleting agent such as rituximab, an interleukin-1 antagonist, or a T cell costimulatory blocking agent such as abatacept. In some embodiments, a multi-specific binding molecule described herein (e.g., an engineered antibody described herein) is useful for treating allergic asthma, allergic rhinitis, hyper-IgE syndrome/Job's syndrome, food allergies, Paroxysmal nocturnal hemoglobinurea (PNH), inflammatory bowel disease and/or other large-organ cytokine-mediated inflammatory conditions.

In some embodiments, an engineered antibody as described herein is used in a method of treating PNH or aHUS. Paroxysmal nocturnal hemoglobinuria (PNH), previously Marchiafava-Micheli syndrome, is a rare, acquired, life-threatening disease of the blood characterized by destruction of red blood cells by the complement system.

In PNH patients, uncontrolled AP activation on the Red Blood Cells (RBC) leads to hemolytic anemia. Approximately 15-30% of patients with PNH have extravascular hemolysis that is unmasked by C5 inhibition and continue to require routine blood transfusions despite therapy. Approximately 1-5% of patients will not respond to some C5 treatments because of polymorphisms in C5 and CR1. Neisseria meningitidis infection can occur in a small number patients treated on some C5 treatments despite vaccination. An unmet medical need exists in complement disorders (e.g., PNH and aHUS) despite the availablility of C5 inhibition therapies.

Without treatement PNH erythrocytes may develop intravascular hemolysis. In the presence of anti-C5 therapy, C3 fragment deposition may lead to breathrough and extravascular hemolysis. C3 opsonization via RES macrophages in the liver and spleen may also occur. Treatement with a Factor D inhibitor may protext PNH erythrocytes from both intravascular and extravascular hemolysis.

In various instances, a multi-specific binding molecule described herein (e.g., an engineered antibody described herein) treats, alleviates, reduces the prevalence of, reduces the frequency of, or reduces the level or amount of one or more symptoms or biomarkers of PNH. Symptoms and biomarkers of PNH include, without limitation, hemolysis, abdominal pain (e.g., severe abdominal pain or stomach pain), leg pain, leg swelling, headaches (e.g., severe headaches), back pain, weakness, fatigue (e.g., tiredness, difficulty performing daily activities, trouble concentrating, dizziness, weakness), shortness of breath, difficulty swallowing, yellowing of the skin, yellowing of the eyes, erectile dysfunction, anemia, pulmonary hypertension, recurrent infection, susceptibility to infection, colored urine (e.g., dark color), Budd-Chiari syndrome, heart palpitations, myelodysplasia, acute leukemia, menorrhagia, lightheadedness, irritability, red blood in urine, thrombosis (e.g., in veins, e.g., hepatic vein thrombosis or sagittal vein thrombosis), smooth muscle dystonias, abdominal contractions, esophageal spasms, chronic renal disease, Ham's acid hemolysis test results, sucrose hemolysis test results, binding of monoclonal antibodies directed against cell-bound complement regulators (CD59, CD24, CD66b, CD16, fluorescently-labeled aerolysin (FLAER)) to a peripheral blood sample, high serum lactate dehydrogenase, serum creatinine levels, fibrinolysis, plasmin-mediated clot degradation, D-dimer levels, and others known in the art. Symptoms may be increased following any of infection, alcohol, exercise, or stress. Specific symptoms and progression of symptoms vary among subjects.

Thus, in some embodiments, a multi-specific binding molecule described herein (e.g., an engineered antibody described herein) is administered to a subject in need thereof, e.g., a subject having Paroxysmal Nocturnal Hemoglobinuria (PNH) or a subject having Atypical Hemolytic Uremic Syndrome (aHUS).

In various instances, administration of a multi-specific binding molecule described herein (e.g., an engineered antibody described herein) results in a decrease in the prevalence, frequency, level, and/or amount of one or more symptoms or biomarkers of PNH as described herein or otherwise known in the art, e.g., a decrease of at least 3%, 4%, 5%, 6%, 7%, 8%, 9%, 10%, 15%, 20%, 25%, 30%, 35%, 40%, 45%, 50%, 60%, 70%, 80%, 90%, 95%, 99%, or 100% of one or more symptoms or biomarkers as compared to a prior measurement in the subject or to a reference value.

In some embodiments, administration of a multi-specific binding molecule described herein (e.g., an engineered antibody described herein) to a subject having PNH results in a greater decrease or improvement in one or more symptoms or biomarkers of PNH than does a reference antibody, e.g., a reference antibody comprising a heavy chain having the amino acid sequence of SEQ ID NO: 1 and a light chain having the amino acid sequence of SEQ ID NO: 2, under comparable conditions. Treatment of subjects having PNH according can result in decreased frequency or likelihood of increased symptoms following any of infection, alcohol, exercise, or stress.

Atypical hemolytic-uremic syndrome (aHUS) is a disease that primarily affects kidney function. This condition, which can occur at any age, can cause abnormal blood clots (thrombi) to form in small blood vessels in the kidneys. These clots can cause serious medical problems if they restrict or block blood flow. Atypical hemolytic-uremic syndrome is characterized by three major features related to abnormal clotting: hemolytic anemia, thrombocytopenia, and kidney failure. Mutations in the genes associated with atypical hemolytic-uremic syndrome lead to uncontrolled activation of the complement system. The overactive system attacks cells that line blood vessels in the kidneys, causing inflammation and the formation of abnormal clots. These abnormalities lead to kidney damage and, in many cases, kidney failure and ESRD.

In various instances, a multi-specific binding molecule described herein (e.g., an engineered antibody described herein) treats, alleviates, reduces the prevalence of, reduces the frequency of, or reduces the level or amount of one or more symptoms or biomarkers of aHUS. Symptoms of aHUS include, without limitation, nausea, vomiting, confusion, shortness of breath (dyspnea), fatigue, anemia, thrombocytopenia, kidney damage, kidney failure, end-stage renal disease, stroke, gastrointestinal issues (e.g., severe stomach pain), colon inflammation, blood vessel damage, heart attacks, neurological issues (e.g., seizures), anemia, hemolysis, pale skin, jaundice, edema, rapid heart rate, yellowing of the eyes, thrombotic microangiopathy (TMA), transplant-associated thrombotic microangiopathy (TA-TMA), stroke, heart attack, malaise, microangiopathic anemia, bloody diarrhea, lung complications, pancreatitis, schistocytes, encephalopathy, coma, malignant hypertension, proteinuria, decreased platelets, decreased hemoglobulin, decreased heptaglobin, increased lactate dehydrogenase (LDH), increased creatine, and/or increased blood urea nitrogen.

In various instances, administration of a multi-specific binding molecule described herein (e.g., an antibody described herein) to a subject having aHUS results in a decrease in the prevalence, frequency, level, or amount of one or more symptoms or biomarkers of aHUS as described herein or otherwise known in the art, e.g., a decrease of at least 3%, 4%, 5%, 6%, 7%, 8%, 9%, 10%, 15%, 20%, 25%, 30%, 35%, 40%, 45%, 50%, 60%, 70%, 80%, 90%, 95%, 99%, or 100% of one or more symptoms as compared to a prior measurement in the subject or to a reference value.

In some embodiments, administration of a multi-specific binding molecule described herein (e.g., an engineered antibody described herein) to a subject having aHUS results in a greater decrease or improvement in one or more symptoms of aHUS than does a reference protein, e.g., a reference antibody comprising a heavy chain having the amino acid sequence of SEQ ID NO: 1 and a light chain having the amino acid sequence of SEQ ID NO: 2, under comparable conditions.

In some embodiments, an engineered antibody as described herein is used in a method of treating acquired microthrombotic diseases. Acquired microthrombotic diseases characterized by thrombocytopenia, hemolytic anemia, renal failure affect 4 per million globally and 20% of hematopoietic stem cell recipients in the US (8000 per year in US). Thrombotic microangiopathy, abbreviated TMA, is a pathology that results in thrombosis in capillaries and arterioles, due to an endothelial injury. These diseases can be a result of genetic complement mutations such as aHUS. They can also be secondary to some medications, stem cell transplant, infection-related, pregnancy, surgery, malignancy, or STEC-associated. It may be seen in association with thrombocytopenia, anemia, purpura and renal failure. The classic TMAs are hemolytic uremic syndrome and thrombotic thrombocytopenic purpura. Other conditions with TMA include atypical hemolytic uremic syndrome, disseminated intravascular coagulation, scleroderma renal crisis, malignant hypertension, antiphospholipid antibody syndrome, and drug toxicities, e.g. calcineurin inhibitor toxicity. TMAs often result in decreased endothelial thromboresistance, leukocyte adhesion to damaged endothelium, complement consumption, enhanced vascular shear stress, and abnormal von Williebrand factor (vWF) fragmentation.

Transplant-associated thrombotic microangiopathy (TA-TMA) can exhibit complications of the transplant itself, including infection, graft-versus-host disease, and disseminated intravascular coagulation, as well as the side effects of immunosuppressive drugs, can mimic a TMA. Because the pathophysiology of TA-TMA is poorly understood, current treatment options are suboptimal, and the condition carries a very high mortality rate. In various instances, a multi-specific binding molecule described herein (e.g., an engineered antibody described herein) treats, alleviates, reduces the prevalence of, reduces the frequency of, or reduces the level or amount of one or more symptoms or biomarkers of TMA or TA-TMA. Symptoms of TA-TMA include, without limitation, fever, microangiopathic hemolytic anemia (see schistocytes in a blood smear), renal failure, thrombocytopenia, neurological manifestations, multi-organ failure or injury is also possible, affecting the brain, kidneys, heart, liver, and other major organs.

The vast majority of TA-TMA patients lack suppression of ADAMTS13 (a disintegrin and metalloproteinase with a thrombospondin type 1 motif, member 13) activity to less than 5% to 10% of normal and do not have a complete response to plasma exchange. In addition, the presentation of TA-TMA is highly heterogeneous, ranging from asymptomatic, low-level red blood cell fragmentation to fulminant disease. The diagnosis of TA-TMA is made most reliably by examination of the peripheral blood film for red blood cell fragments.

In various instances, administration of a multi-specific binding molecule described herein (e.g., an antibody described herein) to a subject having TA-TMA results in a decrease in the prevalence, frequency, level, or amount of one or more symptoms or biomarkers of TA-TMA as described herein or otherwise known in the art, e.g., a decrease of at least 3%, 4%, 5%, 6%, 7%, 8%, 9%, 10%, 15%, 20%, 25%, 30%, 35%, 40%, 45%, 50%, 60%, 70%, 80%, 90%, 95%, 99%, or 100% of one or more symptoms as compared to a prior measurement in the subject or to a reference value.

In some embodiments, administration of a multi-specific binding molecule described herein (e.g., an engineered antibody described herein) to a subject having TA-TMA results in a greater decrease or improvement in one or more symptoms of TA-TMA than does a reference protein, e.g., a reference antibody comprising a heavy chain having the amino acid sequence of SEQ ID NO: 1 and a light chain having the amino acid sequence of SEQ ID NO: 2, under comparable conditions.

In some embodiments, an engineered antibody as described herein is used in a method of treating C3 Glomerulopathies (C3G). C3 Glomerulopathies (C3G) are a group of rare renal diseases characterized by C3 deposition without immunoglobulin deposits. These renal diseases can be subdivided into Dense Deposit Disease (DDD) and C3 glomerulonephritis (C3GN) based on histology. C3G patients have in common mutations in alternate complement pathway genes, the presence of C3 nephritic factors and a substantial risk for both ESRD and recurrence of disease after renal transplant. C3 convertase autoantibodies stabilize the C3 convertase complex and increase local generation of the alternative pathway complement. Kidney failure occurs to 50% of patients within 10 years with limited renal transplant in C3 diseases by 50% recurrence and graft loss post-transplant.

Dense deposit disease (DDD), a very rare kidney disease characterized on a renal biopsy by an abundance C3 in the renal glomeruli, and named for the extremely dense deposits seen in the glomerular basement membrane (GBM) using electron microscopy. In both DDD and C3GN, deposits of C3 and other proteins in the GBM disrupt kidney function. Progressive damage to the glomeruli occurs eventually resulting in kidney failure. When kidney failure occurs, dialysis must be started or transplantation must be performed. The rate of progression to end-stage kidney failure and dialysis appears to be similar for both DDD and C3GN. In addition to dense deposits in the kidney, persons with DDD can develop deposits in the eyes. The signs and symptoms of DDD and C3GN are similar, including but not limited to hematuria, proteinuria, white blood cells in the urine; edema, high blood pressure, decreased urine output; and decreased alertness.

If C3G (either DDD or C3GN) is suspected, immunofluorescence analysis should show abundant C3 in the glomerular capillaries. In addition to C3, the glomeruli may contain other complement system proteins with reduced levels of complement proteins in blood circulation. The causes of complement dysregulation can be genetic variants in complement regulatory proteins and autoantibodies to the convertases [5, 9, 11, 12, 13, 14, 16, 17, 18, 19, 20, 21, 22, 23, 24, 25, 26, 27, 28, 29]; mutations in complement Factor H (CFH), complement Factor I (CFI), MCP (also termed membrane cofactor protein or CD46), complement Factor B (CFB), complement Factor C3 and CFHR5. Complement dysregulation may also be due to acquired factors.

In various instances, administration of a multi-specific binding molecule described herein (e.g., an antibody described herein) to a subject having DDD or C3GN results in a decrease in the prevalence, frequency, level, or amount of one or more symptoms or biomarkers of DDD or C3GN as described herein or otherwise known in the art, e.g., a decrease of at least 3%, 4%, 5%, 6%, 7%, 8%, 9%, 10%, 15%, 20%, 25%, 30%, 35%, 40%, 45%, 50%, 60%, 70%, 80%, 90%, 95%, 99%, or 100% of one or more symptoms as compared to a prior measurement in the subject or to a reference value.

In some embodiments, administration of a multi-specific binding molecule described herein (e.g., an engineered antibody described herein) to a subject having DDD or C3GN results in a greater decrease or improvement in one or more symptoms of DDD or C3GN than does a reference protein, e.g., a reference antibody comprising a heavy chain having the amino acid sequence of SEQ ID NO: 1 and a light chain having the amino acid sequence of SEQ ID NO: 2, under comparable conditions.

In some embodiments, an engineered antibody as described herein is used in a method of treating multiple neuropathy. Complement activation in peripheral neurons during injury results in multiple neuropathy subtypes such as hereditary/familial amyloid neuropathy; Guillain-Barré syndrome (GBS), a demyelinating neuropathy can damage motor, sensory, and autonomic nerve fibers; Diabetic neuropathy; etc. Peripheral neuropathy (PN) can be caused by inflammation of, or damage to, the nerves. It can result in tingling, numbness and burning pain in any part of the body, but commonly is felt in the hands, feet and lower legs. Some patients may experience an increased sensitivity to pain, loss of sensitivity to temperature, sensorimotor impairment due to nerve damage.

The systemic amyloidoses are a diverse group of disorders that can lead to multi-organ dysfunction through the deposition of abnormal amyloid fibrils. Hereditary amyloid peripheral neuropathies can be further classified according to the type of amyloid protein that causes the disease process. These include transthyretin, apoprotein A1, gelsolin, and Aβ2-microglobulin. Mutations in the TTR gene lead to the most common form of inherited amyloidosis, whereas amyloid light chain (AL) amyloidosis is the most common acquired form. Peripheral nervous system involvement is common and may present as a length dependent sensorimotor polyneuropathy, focal neuropathy, multi-focal neuropathy, or autonomic neuropathy. Familial Amyloid Polyneuropathy (FAP) refers to a group of hereditary amyloidoses which typically have prominent clinical manifestations involving the peripheral sensorimotor and/or autonomic nervous system.

Guillain-Barré syndrome (GBS) is a rapid-onset muscle weakness caused by the immune system damaging the peripheral nervous system. The initial symptoms are typically changes in sensation or pain along with muscle weakness, beginning in the feet and hands spreads to the arms and upper body with both sides being involved. Symptoms of Guillain-Barre syndrome often begins with tingling and weakness starting in the feet and legs, spreading to the upper body and arms, difficulty with eye or facial movements, including speaking, chewing or swallowing, severe pain, difficulty with bladder control or bowel function, rapid heart rate, low or high blood pressure and difficulty breathing. As GBS progresses, muscle weakness can evolve into paralysis. Guillain-Barre syndrome is now known to occur in several forms. The main types are Acute inflammatory demyelinating polyradiculoneuropathy (AIDP), Miller Fisher syndrome (MFS), Acute motor axonal neuropathy (AMAN) and acute motor-sensory axonal neuropathy (AMSAN).

Diabetic neuropathies are a family of nerve disorders caused by diabetes. People with diabetes can, over time, develop nerve damage throughout the body. Some people with nerve damage have no symptoms. Others may have symptoms such as pain, tingling, or numbness, loss of feeling in the hands, arms, feet, and legs. Nerve problems can occur in every organ system, including the digestive tract, heart, and sex organs. Symptoms of nerve damage may include numbness, tingling, or pain in the toes, feet, legs, hands, arms, and fingers, muscle wasting, indigestion, nausea, or vomiting diarrhea or constipation, dizziness or faintness due to a drop in blood pressure after standing or sitting up, problems with urination, erectile dysfunction in men or vaginal dryness in women.

In various instances, administration of a multi-specific binding molecule described herein (e.g., an antibody described herein) to a subject having a peripheral neuropathy results in a decrease in the prevalence, frequency, level, or amount of one or more symptoms or biomarkers of a peripheral neuropathy as described herein or otherwise known in the art, e.g., a decrease of at least 3%, 4%, 5%, 6%, 7%, 8%, 9%, 10%, 15%, 20%, 25%, 30%, 35%, 40%, 45%, 50%, 60%, 70%, 80%, 90%, 95%, 99%, or 100% of one or more symptoms as compared to a prior measurement in the subject or to a reference value.

In some embodiments, administration of a multi-specific binding molecule described herein (e.g., an engineered antibody described herein) to a subject having a peripheral neuropathy results in a greater decrease or improvement in one or more symptoms of a peripheral neuropathy than does a reference protein, e.g., a reference antibody comprising a heavy chain having the amino acid sequence of SEQ ID NO: 1 and a light chain having the amino acid sequence of SEQ ID NO: 2, under comparable conditions.

In some embodiments, a multi-specific binding molecule described herein (e.g., an engineered antibody as described herein) exhibits a decreased effective dose as compared to a reference protein (e.g., a reference antibody comprising a heavy chain having the amino acid sequence of SEQ ID NO: 1 and a light chain having the amino acid sequence of SEQ ID NO: 2). For instance, an effective dose of an engineered antibody as described herein may be, e.g., less than 1,000 mg/dose, e.g., less than 900 mg/dose, 800 mg/dose, 700 mg/dose, 600 mg/dose, 500 mg/dose, 550 mg/dose, 400 mg/dose, 350 mg/dose, 300 mg/dose, 200 mg/dose, 100 mg/dose, 50 mg/dose, 25 mg/dose, or less. In certain instances, an effective dose of an engineered antibody as disclosed herein is lower than an effective or recommended or approved dosage of a reference antibody, e.g., a reference antibody comprising a heavy chain having the amino acid sequence of SEQ ID NO: 1 and a light chain having the amino acid sequence of SEQ ID NO: 2, which dosage of a reference antibody may be, e.g., 900 mg/dose or 600 mg/dose. Alternatively or in combination with a dosage as disclosed herein, an engineered antibody as described herein may be effectively or usefully administered at a frequency that is less than once per week, e.g., less than once every week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months, 7 months, 8 months, 9 months, 10 months, 11 months, or year. In certain instances, an effective or useful administration frequency of an engineered antibody as disclosed herein is lower than an effective or recommended or approved administration frequency of a reference antibody, e.g., a reference antibody comprising a heavy chain having the amino acid sequence of SEQ ID NO: 1 and a light chain having the amino acid sequence of SEQ ID NO: 2, which administration frequency can be administered weekly (e.g., at a dosage of 300-600 mg, depending on weight of subject) or every two weeks (e.g., at a dosage of 300-1200 mg, depending on weight of subject).

In some embodiments, a multi-specific binding molecule described herein (e.g., an engineered antibody described herein) can be administered at a decreased dose amount as compared to a reference protein, e.g., a reference antibody comprising a heavy chain having the amino acid sequence of SEQ ID NO: 1 and a light chain having the amino acid sequence of SEQ ID NO: 2, while achieving an equal, equally effective, comparably effective, or substantially effective outcome, where the engineered antibody is administered in an identical, equivalent, or substantially equivalent formulation and/or by an identical, equivalent, or substantially equivalent route of administration as the reference (e.g., a reference antibody comprising a heavy chain having the amino acid sequence of SEQ ID NO: 1 and a light chain having the amino acid sequence of SEQ ID NO: 2). In some embodiments, an engineered antibody described herein can be administered at an increased interval as compared to a reference antibody (e.g., a reference antibody comprising a heavy chain having the amino acid sequence of SEQ ID NO: 1 and a light chain having the amino acid sequence of SEQ ID NO: 2) while achieving an equal, equally effective, comparably effective, or substantially effective outcome, where the engineered antibody is administered in an identical, equivalent, or substantially equivalent formulation and/or by an identical, equivalent, or substantially equivalent route of administration as the reference (e.g., a reference antibody comprising a heavy chain having the amino acid sequence of SEQ ID NO: 1 and a light chain having the amino acid sequence of SEQ ID NO: 2). In some embodiments, an engineered antibody described herein can be administered in a decreased number of unit dosages, and/or for a decreased period of treatment, as compared to a reference antibody (e.g., a reference antibody comprising a heavy chain having the amino acid sequence of SEQ ID NO: 1 and a light chain having the amino acid sequence of SEQ ID NO: 2) while achieving an equal, equally effective, comparably effective, or substantially effective outcome, where the engineered antibody is administered in an identical, equivalent, or substantially equivalent formulation and/or by an identical, equivalent, or substantially equivalent route of administration as the reference (e.g., a reference antibody comprising a heavy chain having the amino acid sequence of SEQ ID NO: 1 and a light chain having the amino acid sequence of SEQ ID NO: 2).

In accordance with some such embodiments, an administered dose of an engineered antibody described herein may be less likely to elicit an adverse response when administered to a subject, e.g., an adverse immune response, than would an effective dose of a reference antibody, e.g., a reference antibody comprising a heavy chain having the amino acid sequence of SEQ ID NO: 1 and a light chain having the amino acid sequence of SEQ ID NO: 2. Accordingly, in various embodiments, an engineered antibody as disclosed herein may be less likely than a reference antibody, e.g., a reference antibody comprising a heavy chain having the amino acid sequence of SEQ ID NO: 1 and a light chain having the amino acid sequence of SEQ ID NO: 2, per unit of activity administered to induce an adverse reaction or side effect. In various embodiments, an engineered antibody as disclosed herein may less likely than a reference antibody, e.g., a reference antibody comprising a heavy chain having the amino acid sequence of SEQ ID NO: 1 and a light chain having the amino acid sequence of SEQ ID NO: 2, per unit of activity administered, to induce an adverse reaction or side effect having a particular degree of severity. In various embodiments, an engineered antibody as disclosed herein may induce one or more adverse reactions or side effects to a lesser degree or in fewer patients than a reference antibody, e.g., a reference antibody comprising a heavy chain having the amino acid sequence of SEQ ID NO: 1 and a light chain having the amino acid sequence of SEQ ID NO: 2, per unit of activity administered. Examples of adverse reactions or side effects that may be associated with the administration of an antibody capable of binding CFD, e.g., a prior antibody such as a reference antibody comprising a heavy chain having the amino acid sequence of SEQ ID NO: 1 and a light chain having the amino acid sequence of SEQ ID NO: 2, can include headache, nasopharyngitis, back pain, nausea, diarrhea, hypertension, upper respiratory infection, abdominal pain, vomiting, anemia, cough, peripheral edema, and/or urinary tract infection.

In some embodiments, administration of a multi-specific binding molecule described herein (e.g., an engineered antibody described herein) decreases CFD titer in serum. A typical concentration of CFD in human plasma is about 0.37 μM. Upon administration of one or more doses of an engineered antibody as disclosed herein, the concentration of human CFD in plasma may be reduced in a subject as compared to a prior measured concentration in the same subject or as compared to a standard value, e.g., as compared to a value of about 0.37 μM. In various instances, a concentration of CFD in serum following administration of an engineered antibody, including any number of doses (e.g., 1 dose, 3 doses, or a number of doses prescribed over a period of months or years) administered over any period of time (e.g., 1-4 weeks, 1-12 months, or 1-3 or more years) in any of one or more subjects or an aggregate of subjects, may be equal to or less than, e.g., 0.35 μM, 0.325 μM, 0.30 μM, 0.275 μM, 0.25 μM, 0.225 μM, 0.20 μM, 0.175 μM, 0.15 μM, 0.125 μM, 0.10 μM, 0.075 μM, 0.05 μM, or 0.025 μM. In some embodiments, upon administration to a subject, a multi-specific binding molecule described herein (e.g., an engineered antibody described herein) results in a greater decrease in CFD titer in serum that does a reference protein, e.g., a reference antibody comprising a heavy chain having the amino acid sequence of SEQ ID NO: 1 and a light chain having the amino acid sequence of SEQ ID NO: 2 a reference antibody comprising a heavy chain having the amino acid sequence of SEQ ID NO: 1 and a light chain having the amino acid sequence of SEQ ID NO: 2, under comparable conditions.

In some embodiments, upon administration to a subject (e.g., at a single dose), a multi-specific binding molecule described herein (e.g., an engineered antibody described herein) is measured at an increased level in plasma at a defined time following administration (e.g., 1, 2, 3, 4, 5, 6, 7, 8, 9, 10, 11, 12, 13, 14, or more days), relative to level of a control at the same defined time (e.g., a reference protein, e.g., a reference antibody comprising a heavy chain having the amino acid sequence of SEQ ID NO: 1 and a light chain having the amino acid sequence of SEQ ID NO: 2). For example, at a defined time following administration of a single dose, a level of an engineered antibody described herein is at least 10%, 20%, 30%, 40%, 50%, 60%, 70%, 80%, 90%, 100%, 150%, 200%, 300%, 400%, or 500% higher than a corresponding level of a reference antibody, e.g., a reference antibody comprising a heavy chain having the amino acid sequence of SEQ ID NO: 1 and a light chain having the amino acid sequence of SEQ ID NO: 2.

In some embodiments, a multi-specific binding molecule described herein (e.g., an engineered antibody described herein) is measured at an increased level in plasma at a defined time (e.g., 1, 2, 3, 4, 5, 6, 7, 8, 9, 10, 11, 12, 13, 14, or more days) following administration (e.g., of a single dose), relative to level of a control at the same defined time (e.g., a reference protein, e.g., a reference antibody comprising a heavy chain having the amino acid sequence of SEQ ID NO: 1 and a light chain having the amino acid sequence of SEQ ID NO: 2). For example, at a defined time following administration, a level of an engineered antibody described herein is at least 10%, 20%, 30%, 40%, 50%, 60%, 70%, 80%, 90%, 100%, 150%, 200%, 300%, 400%, or 500% higher than a corresponding level of a reference antibody (e.g., a reference antibody comprising a heavy chain having the amino acid sequence of SEQ ID NO: 1 and a light chain having the amino acid sequence of SEQ ID NO: 2).

In some embodiments, an engineered antibody described herein has increased half-life (e.g., relative to a control, e.g., a reference antibody, e.g., a reference antibody comprising a heavy chain having the amino acid sequence of SEQ ID NO: 1 and a light chain having the amino acid sequence of SEQ ID NO: 2), and thus the engineered antibody can be administered to a subject at increased inter-dose intervals. For example, an engineered antibody can be administered once every week, every two weeks, every three weeks, every four weeks, every 6 weeks, every 8 weeks, or longer duration.

In some embodiments, a therapeutically effective amount of a multi-specific binding molecule described herein (e.g., an engineered antibody described herein) is about 90%, 80%, 70%, 60%, 50%, 40%, 30%, 20%, 10%, or 5% of an effective amount of a reference therapeutic protein, e.g., a reference antibody comprising a heavy chain having the amino acid sequence of SEQ ID NO: 1 and a light chain having the amino acid sequence of SEQ ID NO: 2. In some embodiments, a single dose of an engineered antibody described herein achieves a comparable therapeutic effect as two or more doses of a reference antibody, e.g., a reference antibody comprising a heavy chain having the amino acid sequence of SEQ ID NO: 1 and a light chain having the amino acid sequence of SEQ ID NO: 2.

In some embodiments, a multi-specific binding molecule described herein (e.g., an engineered antibody described herein) is administered at a dose that is about 90%, 80%, 70%, 60%, 50%, 40%, 30%, 20%, 10%, or 5% of the concentration of a target antigen (e.g., CFD) in the serum of the subject.

In some embodiments, a multi-specific binding molecule described herein (e.g., an engineered antibody described herein) can be administered by a route other than intravenous administration, e.g., by subcutaneous administration. Thus, in various embodiments, an antibody as disclosed herein can be administered subcutaneously. In some embodiments, an engineered antibody described herein can be administered by intravenous and subcutaneous routes, e.g., as components of single treatment strategy. Intravenous and subcutaneous administration may be concurrent or non-concurrent.

In some embodiments, a multi-specific binding molecule described herein (e.g., an engineered antibody described herein) can be used in a number of diagnostic and therapeutic applications. For example, detectably-labeled versions of engineered antibodies as described herein can be used in assays to detect the presence or amount of the CFD in a sample (e.g., a biological sample). Engineered antibodies described herein can be used in in vitro assays for studying inhibition of CFD activity and/or cleavage. In some embodiments, an engineered antibody described herein can be used as a positive control in an assay designed to identify additional novel compounds that inhibit complement activity or otherwise are useful for treating a complement-associated disorder. For example, an engineered antibody described herein can be used as a positive control in an assay to identify additional compounds (e.g., small molecules, aptamers, or antibodies) that reduce or abrogate CFD production or formation of MAC.

Multi-specific binding molecules described herein (e.g., engineered antibodies as described herein) may be used in monitoring a subject, e.g., a subject having, suspected of having, at risk of developing, or under treatment for one or more complement-associated conditions. Monitoring may include determining the amount or activity of CFD in a subject, e.g., in the serum of a subject. In some embodiments, the evaluation is performed at least one (1) hour, e.g., at least 2, 4, 6, 8, 12, 24, or 48 hours, or at least 1 day, 2 days, 4 days, 10 days, 13 days, 20 days or more, or at least 1 week, 2 weeks, 4 weeks, 10 weeks, 13 weeks, 20 weeks or more, after an administration of an engineered antibody as described herein. The subject can be evaluated in one or more of the following periods: prior to beginning of treatment; during the treatment; or after one or more elements of the treatment have been administered. Evaluation can include evaluating the need for further treatment, e.g., evaluating whether a dosage, frequency of administration, or duration of treatment should be altered. It can also include evaluating the need to add or drop a selected therapeutic modality, e.g., adding or dropping any of the treatments for a complement-associated disorder described herein.

### Formulations and Administration

In various embodiments, a multi-specific binding molecule described herein (e.g., an engineered antibody described herein) can be incorporated into a pharmaceutical composition. Such a pharmaceutical composition can be useful, e.g., for the prevention and/or treatment of diseases, e.g., PNH and/or aHUS, or other complement-associated disorder. Pharmaceutical compositions can be formulated by methods known to those skilled in the art (such as described in Remington's Pharmaceutical Sciences, 17th edition, ed. Alfonso R. Gennaro, Mack Publishing Company, Easton, Pa. (1985)).

A suitable means of administration can be selected based on the age and condition of a subject. A single dose of the pharmaceutical composition containing a multi-specific binding molecule described herein (e.g., an engineered antibody described herein) can be selected from a range of 0.001 to 1000 mg/kg of body weight. On the other hand, a dose can be selected in the range of 0.001 to 100000 mg/body weight, but the present disclosure is not limited to such ranges. The dose and method of administration varies depending on the weight, age, condition, and the like of the patient, and can be suitably selected as needed by those skilled in the art.

In various instances, a pharmaceutical composition can be formulated to include a pharmaceutically acceptable carrier or excipient. Examples of pharmaceutically acceptable carriers include, without limitation, any and all solvents, dispersion media, coatings, antibacterial and antifungal agents, isotonic and absorption delaying agents, and the like that are physiologically compatible. Compositions of the present invention can include a pharmaceutically acceptable salt, e.g., an acid addition salt or a base addition salt.

In various embodiments, a composition including an antibody as described herein, e.g., a sterile formulation for injection, can be formulated in accordance with conventional pharmaceutical practices using distilled water for injection as a vehicle. For example, physiological saline or an isotonic solution containing glucose and other supplements such as D-sorbitol, D-mannose, D-mannitol, and sodium chloride may be used as an aqueous solution for injection, optionally in combination with a suitable solubilizing agent, for example, alcohol such as ethanol and polyalcohol such as propylene glycol or polyethylene glycol, and a nonionic surfactant such as polysorbate 80™, HCO-50 and the like.

As disclosed herein, a pharmaceutical composition may be in any form known in the art. Such forms include, e.g., liquid, semi-solid and solid dosage forms, such as liquid solutions (e.g., injectable and infusible solutions), dispersions or suspensions, tablets, pills, powders, liposomes and suppositories.

Selection or use of any particular form may depend, in part, on the intended mode of administration and therapeutic application. For example, compositions containing a composition intended for systemic or local delivery can be in the form of injectable or infusible solutions. Accordingly, the compositions can be formulated for administration by a parenteral mode (e.g., intravenous, subcutaneous, intraperitoneal, or intramuscular injection). As used herein, parenteral administration refers to modes of administration other than enteral and topical administration, usually by injection, and include, without limitation, intravenous, intranasal, intraocular, pulmonary, intramuscular, intraarterial, intrathecal, intracapsular, intraorbital, intracardiac, intradermal, intrapulmonary, intraperitoneal, transtracheal, subcutaneous, subcuticular, intraarticular, subcapsular, subarachnoid, intraspinal, epidural, intracerebral, intracranial, intracarotid and intrasternal injection and infusion.

Route of administration can be parenteral, for example, administration by injection, transnasal administration, transpulmonary administration, or transcutaneous administration. Administration can be systemic or local by intravenous injection, intramuscular injection, intraperitoneal injection, subcutaneous injection.

In various embodiments, a pharmaceutical composition of the present invention can be formulated as a solution, microemulsion, dispersion, liposome, or other ordered structure suitable for stable storage at high concentration. Sterile injectable solutions can be prepared by incorporating a composition described herein in the required amount in an appropriate solvent with one or a combination of ingredients enumerated above, as required, followed by filter sterilization. Generally, dispersions are prepared by incorporating a composition described herein into a sterile vehicle that contains a basic dispersion medium and the required other ingredients from those enumerated above. In the case of sterile powders for the preparation of sterile injectable solutions, methods for preparation include vacuum drying and freeze-drying that yield a powder of a composition described herein plus any additional desired ingredient (see below) from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition a reagent that delays absorption, for example, monostearate salts, and gelatin.

A pharmaceutical composition can be administered parenterally in the form of an injectable formulation comprising a sterile solution or suspension in water or another pharmaceutically acceptable liquid. For example, the pharmaceutical composition can be formulated by suitably combining the therapeutic molecule with pharmaceutically acceptable vehicles or media, such as sterile water and physiological saline, vegetable oil, emulsifier, suspension agent, surfactant, stabilizer, flavoring excipient, diluent, vehicle, preservative, binder, followed by mixing in a unit dose form required for generally accepted pharmaceutical practices. The amount of active ingredient included in the pharmaceutical preparations is such that a suitable dose within the designated range is provided. Nonlimiting examples of oily liquid include sesame oil and soybean oil, and it may be combined with benzyl benzoate or benzyl alcohol as a solubilizing agent. Other items that may be included are a buffer such as a phosphate buffer, or sodium acetate buffer, a soothing agent such as procaine hydrochloride, a stabilizer such as benzyl alcohol or phenol, and an antioxidant. The formulated injection can be packaged in a suitable ampule.

In some embodiments, a composition can be formulated for storage at a temperature below 0° C. (e.g., −20° C. or −80° C.). In some embodiments, the composition can be formulated for storage for up to 2 years (e.g., one month, two months, three months, four months, five months, six months, seven months, eight months, nine months, 10 months, 11 months, 1 year, 1½ years, or 2 years) at 2-8° C. (e.g., 4° C.). Thus, in some embodiments, the compositions described herein are stable in storage for at least 1 year at 2-8° C. (e.g., 4° C.).

In particular instances, a pharmaceutical composition can be formulated as a solution. In some embodiments, a composition can be formulated, for example, as a buffered solution at a suitable concentration and suitable for storage at 2-8° C. (e.g., 4° C.).

Compositions including one or more engineered antibodies as described herein can be formulated in immunoliposome compositions. Such formulations can be prepared by methods known in the art. Liposomes with enhanced circulation time are disclosed in, e.g., U.S. Pat. No. 5,013,556.

In certain embodiments, compositions can be formulated with a carrier that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are known in the art. See, e.g., J. R. Robinson (1978) “Sustained and Controlled Release Drug Delivery Systems,” Marcel Dekker, Inc., New York.

In some embodiments, compositions can be formulated in a composition suitable for intrapulmonary administration (e.g., for administration via an inhaler or nebulizer) to a mammal such as a human. Methods for formulating such compositions are well known in the art. Dry powder inhaler formulations and suitable systems for administration of the formulations are also known in the art. Pulmonary administration may be oral and/or nasal. Examples of pharmaceutical devices for pulmonary delivery include metered dose inhalers, dry powder inhalers (DPIs), and nebulizers. For example, a composition described herein can be administered to the lungs of a subject by way of a dry powder inhaler. These inhalers are propellant-free devices that deliver dispersible and stable dry powder formulations to the lungs. Dry powder inhalers are well known in the art of medicine and include, without limitation: the TURBOHALER® (AstraZeneca; London, England) the AIR® inhaler (ALKERMES®; Cambridge, Mass.); ROTAHALER® (GlaxoSmithKline; London, England); and ECLIPSE™ (Sanofi-Aventis; Paris, France). See also, e.g., PCT Publication Nos. WO 04/026380, WO 04/024156, and WO 01/78693. DPI devices have been used for pulmonary administration of polypeptides such as insulin and growth hormone. In some embodiments, a composition described herein can be intrapulmonarily administered by way of a metered dose inhaler. These inhalers rely on a propellant to deliver a discrete dose of a compound to the lungs. Additional devices and intrapulmonary administration methods are set forth in, e.g., U.S. Patent Application Publication Nos. 20050271660 and 20090110679, the disclosures of each of which are incorporated herein by reference in their entirety.

In some embodiments, compositions can be formulated for delivery to the eye, e.g., in the form of a pharmaceutically acceptable solution, suspension or ointment. A preparation for use in treating an eye can be in the form of a sterile aqueous solution containing, e.g., additional ingredients such as, but not limited to, preservatives, buffers, tonicity agents, antioxidants and stabilizers, nonionic wetting or clarifying agents, and viscosity-increasing agents. A preparation as described herein can be administered topically to the eye of the subject in need of treatment (e.g., a subject afflicted with AMD) by conventional methods, e.g., in the form of drops, or by bathing the eye in a therapeutic solution, containing one or more compositions.

A variety of devices for introducing drugs into the vitreal cavity of the eye may be appropriate, in certain embodiments, for administration of a composition as described herein. For example, U.S. Publication No. 2002/0026176 describes a pharmaceutical-containing plug that can be inserted through the sclera such that it projects into the vitreous cavity to deliver the pharmaceutical agent into the vitreous cavity. In another example, U.S. Pat. No. 5,443,505 describes an implantable device for introduction into a suprachoroidal space or an avascular region for sustained release of drug into the interior of the eye. U.S. Pat. Nos. 5,773,019 and 6,001,386 each disclose an implantable drug delivery device attachable to the scleral surface of an eye. Additional methods and devices (e.g., a transscleral patch and delivery via contact lenses) for delivery of a therapeutic agent to the eye are described in, e.g., Ambati and Adamis (2002) Prog Retin Eye Res 21(2):145-151; Ranta and Urtti (2006) Adv Drug Delivery Rev 58(11):1164-1181; Barocas and Balachandran (2008) Expert Opin Drug Delivery 5(1):1-10(10); Gulsen and Chauhan (2004) Invest Opthalmol Vis Sci 45:2342-2347; Kim et al. (2007) Ophthalmic Res 39:244-254; and PCT publication no. WO 04/073551, the disclosures of which are incorporated herein by reference in their entirety.

In certain embodiments, administration of an antibody as described herein is achieved by administering to a subject a nucleic acid encoding the antibody. Nucleic acids encoding a therapeutic antibody described herein can be incorporated into a gene construct to be used as a part of a gene therapy protocol to deliver nucleic acids that can be used to express and produce antibody within cells. Expression constructs of such components may be administered in any therapeutically effective carrier, e.g. any formulation or composition capable of effectively delivering the component gene to cells in vivo. Approaches include insertion of the subject gene in viral vectors including recombinant retroviruses, adenovirus, adeno-associated virus, lentivirus, and herpes simplex virus-1 (HSV-1), or recombinant bacterial or eukaryotic plasmids. Viral vectors can transfect cells directly; plasmid DNA can be delivered with the help of, for example, cationic liposomes (lipofectin) or derivatized, polylysine conjugates, gramicidin S, artificial viral envelopes or other such intracellular carriers, as well as direct injection of the gene construct or CaPO4 precipitation (see, e.g., WO04/060407). Examples of suitable retroviruses include pLJ, pZIP, pWE and pEM which are known to those skilled in the art (see, e.g., Eglitis et al. (1985) Science 230:1395-1398; Danos and Mulligan (1988) Proc Natl Acad Sci USA 85:6460-6464; Wilson et al. (1988) Proc Natl Acad Sci USA 85:3014-3018; Armentano et al. (1990) Proc Natl Acad Sci USA 87:6141-6145; Huber et al. (1991) Proc Natl Acad Sci USA 88:8039-8043; Ferry et al. (1991) Proc Natl Acad Sci USA 88:8377-8381; Chowdhury et al. (1991) Science 254:1802-1805; van Beusechem et al. (1992) Proc Natl Acad Sci USA 89:7640-7644; Kay et al. (1992) Human Gene Therapy 3:641-647; Dai et al. (1992) Proc Natl Acad Sci USA 89:10892-10895; Hwu et al. (1993) J Immunol 150:4104-4115; U.S. Pat. Nos. 4,868,116 and 4,980,286; and PCT Publication Nos. WO89/07136, WO89/02468, WO89/05345, and WO92/07573). Another viral gene delivery system utilizes adenovirus-derived vectors (see, e.g., Berkner et al. (1988) BioTechniques 6:616; Rosenfeld et al. (1991) Science 252:431-434; and Rosenfeld et al. (1992) Cell 68:143-155). Suitable adenoviral vectors derived from the adenovirus strain Ad type 5 d1324 or other strains of adenovirus (e.g., Ad2, Ad3, Ad7, etc.) are known to those skilled in the art. Yet another viral vector system useful for delivery of the subject gene is the adeno-associated virus (AAV). See, e.g., Flotte et al. (1992) Am J Respir Cell Mol Biol 7:349-356; Samulski et al. (1989) J Virol 63:3822-3828; and McLaughlin et al. (1989) J Virol 62:1963-1973.

In various embodiments, subcutaneous administration can be accomplished by means of a device, such as a syringe, a prefilled syringe, an auto-injector (e.g., disposable or reusable), a pen injector, a patch injector, a wearable injector, an ambulatory syringe infusion pump with subcutaneous infusion sets, or other device for combining with antibody drug for subcutaneous injection.

An injection system of the present disclosure may employ a delivery pen as described in U.S. Pat. No. 5,308,341. Pen devices, most commonly used for self-delivery of insulin to patients with diabetes, are well known in the art. Such devices can comprise at least one injection needle (e.g., a 31 gauge needle of about 5 to 8 mm in length), are typically pre-filled with one or more therapeutic unit doses of a therapeutic solution, and are useful for rapidly delivering solution to a subject with as little pain as possible. One medication delivery pen includes a vial holder into which a vial of a therapeutic or other medication may be received. The pen may be an entirely mechanical device or it may be combined with electronic circuitry to accurately set and/or indicate the dosage of medication that is injected into the user. See, e.g., U.S. Pat. No. 6,192,891. In some embodiments, the needle of the pen device is disposable and the kits include one or more disposable replacement needles. Pen devices suitable for delivery of any one of the presently featured compositions are also described in, e.g., U.S. Pat. Nos. 6,277,099; 6,200,296; and 6,146,361, the disclosures of each of which are incorporated herein by reference in their entirety. A microneedle-based pen device is described in, e.g., U.S. Pat. No. 7,556,615, the disclosure of which is incorporated herein by reference in its entirety. See also the Precision Pen Injector (PPI) device, MOLLY™, manufactured by Scandinavian Health Ltd.

In some embodiments, a composition described herein can be therapeutically delivered to a subject by way of local administration. As used herein, “local administration” or “local delivery,” can refer to delivery that does not rely upon transport of the composition or agent to its intended target tissue or site via the vascular system. For example, the composition may be delivered by injection or implantation of the composition or agent or by injection or implantation of a device containing the composition or agent. In certain embodiments, following local administration in the vicinity of a target tissue or site, the composition or agent, or one or more components thereof, may diffuse to an intended target tissue or site that is not the site of administration.

In some embodiments, a composition described herein can be locally administered to a joint (e.g., an articulated joint). For example, in embodiments where the disorder is arthritis, a therapeutically appropriate composition can be administered directly to a joint (e.g., into a joint space) or in the vicinity of a joint. Examples of intraarticular joints to which a composition described herein can be locally administered include, e.g., the hip, knee, elbow, wrist, sternoclavicular, temperomandibular, carpal, tarsal, ankle, and any other joint subject to arthritic conditions. A composition described herein can also be administered to bursa such as, e.g., acromial, bicipitoradial, cubitoradial, deltoid, infrapatellar, ischial, and any other bursa known in the art of medicine.

In some embodiments, the compositions provided herein are present in unit dosage form, which unit dosage form can be suitable for self-administration. Such a unit dosage form may be provided within a container, typically, for example, a vial, cartridge, prefilled syringe or disposable pen. A doser such as the doser device described in U.S. Pat. No. 6,302,855, may also be used, for example, with an injection system as described herein.

A suitable dose of a composition described herein, which dose is capable of treating or preventing a disorder in a subject, can depend on a variety of factors including, e.g., the age, sex, and weight of a subject to be treated and the particular inhibitor compound used. For example, a different dose of one composition including an antibody as described herein may be required to treat a subject with RA as compared to the dose of a different formulation of that antibody. Other factors affecting the dose administered to the subject include, e.g., the type or severity of the disorder. For example, a subject having RA may require administration of a different dosage than a subject with PNH. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject can also be adjusted based upon the judgment of the treating medical practitioner.

A composition described herein can be administered as a fixed dose, or in a milligram per kilogram (mg/kg) dose. In some embodiments, the dose can also be chosen to reduce or avoid production of antibodies or other host immune responses against one or more of the antigen-binding molecules in the composition. While in no way intended to be limiting, exemplary dosages of an antibody, such as a composition described herein include, e.g., 1-1000 mg/kg, 1-100 mg/kg, 0.5-50 mg/kg, 0.1-100 mg/kg, 0.5-25 mg/kg, 1-20 mg/kg, and 1-10 mg/kg. Exemplary dosages of a composition described herein include, without limitation, 0.1 mg/kg, 0.5 mg/kg, 1.0 mg/kg, 2.0 mg/kg, 4 mg/kg, 8 mg/kg, or 20 mg/kg.

A pharmaceutical solution can include a therapeutically effective amount of a composition described herein. Such effective amounts can be readily determined by one of ordinary skill in the art based, in part, on the effect of the administered composition, or the combinatorial effect of the composition and one or more additional active agents, if more than one agent is used. A therapeutically effective amount of a composition described herein can also vary according to factors such as the disease state, age, sex, and weight of the individual, and the ability of the composition (and one or more additional active agents) to elicit a desired response in the individual, e.g., amelioration of at least one condition parameter, e.g., amelioration of at least one symptom of the complement-mediated disorder. For example, a therapeutically effective amount of a composition described herein can inhibit (lessen the severity of or eliminate the occurrence of) and/or prevent a particular disorder, and/or any one of the symptoms of the particular disorder known in the art or described herein. A therapeutically effective amount is also one in which any toxic or detrimental effects of the composition are outweighed by the therapeutically beneficial effects.

Suitable human doses of any of the compositions described herein can further be evaluated in, e.g., Phase I dose escalation studies. See, e.g., van Gurp et al. (2008) Am J Transplantation 8(8):1711-1718; Hanouska et al. (2007) Clin Cancer Res 13(2, part 1):523-531; and Hetherington et al. (2006) Antimicrobial Agents and Chemotherapy 50(10): 3499-3500.

Toxicity and therapeutic efficacy of compositions can be determined by known pharmaceutical procedures in cell cultures or experimental animals (e.g., animal models of any of the complement-mediated disorders described herein). These procedures can be used, e.g., for determining the LDso (the dose lethal to 50% of the population) and the EDso (the dose therapeutically effective in 50% of the population). The dose ratio between toxic and therapeutic effects is the therapeutic index and it can be expressed as the ratio LDso/EDso. A composition described herein that exhibits a high therapeutic index is preferred. While compositions that exhibit toxic side effects may be used, care should be taken to design a delivery system that targets such compounds to the site of affected tissue and to minimize potential damage to normal cells and, thereby, reduce side effects.

Those of skill in the art will appreciate that data obtained from cell culture assays and animal studies can be used in formulating a range of dosage for use in humans. Appropriate dosages of compositions described herein lie generally within a range of circulating concentrations of the compositions that include the EDso with little or no toxicity. The dosage may vary within this range depending upon the dosage form employed and the route of administration utilized. For a composition described herein, the therapeutically effective dose can be estimated initially from cell culture assays. A dose can be formulated in animal models to achieve a circulating plasma concentration range that includes the ICFDo (i.e., the concentration of the antibody which achieves a half-maximal inhibition of symptoms) as determined in cell culture. Such information can be used to more accurately determine useful doses in humans. Levels in plasma may be measured, for example, by high performance liquid chromatography. In some embodiments, e.g., where local administration (e.g., to the eye or a joint) is desired, cell culture or animal modeling can be used to determine a dose required to achieve a therapeutically effective concentration within the local site.

### Combination Therapies

In various embodiments, an engineered antibody as described herein may be included in a course of treatment that further includes administration of at least one additional agent to a subject. In various instances, an additional agent administered in combination with an engineered antibody as described herein may be an agent that inhibits complement, e.g., an agent that inhibits terminal compliment. In various instances, an additional agent administered in combination with an antibody as described herein may be an agent that inhibits inflammation. In various instances, an additional agent administered in combination with an antibody as described herein may be an agent that treats a symptom of PNH. In various instances, an additional agent administered in combination with an antibody as described herein may be an agent that treats a symptom of aHUS.

In some embodiments, the methods can be performed in conjunction with other therapies for complement-associated disorders. For example, the composition can be administered to a subject at the same time, prior to, or after, plasmapheresis, IVIG therapy, or plasma exchange. See, e.g., Appel et al. (2005) J Am Soc Nephrol 16:1392-1404. In some embodiments, the composition can be administered to a subject at the same time, prior to, or after, a kidney transplant.

In various instances, an additional agent administered in combination with an engineered antibody as described herein may be administered at the same time as an engineered antibody, on the same day as an engineered antibody, or in the same week as an engineered antibody. In various instances, an additional agent administered in combination with an engineered antibody as described herein may be administered in a single formulation with an engineered antibody. In certain embodiments, an additional agent administered in a manner temporally separated from administration of an engineered antibody as described herein, e.g., one or more hours before or after, one or more days before or after, one or more weeks before or after, or one or more months before or after administration of an engineered antibody. In various embodiments, the administration frequency of one or more additional agents may be the same as, similar to, or different from the administration frequency of an engineered antibody as described herein.

Encompassed within combination therapy is the a treatment regimen that includes administration of two distinct antibodies as described herein and/or a treatment regimen that includes administration of an antibody as described herein by a plurality of formulations and/or routes of administration.

In some embodiments, compositions can be formulated with one or more additional therapeutic agents, e.g., additional therapies for treating or preventing a complement-associated disorder (e.g., an AP-associated disorder or a CP-associated disorder) in a subject. Additional agents for treating a complement-associated disorder in a subject will vary depending on the particular disorder being treated, but can include, without limitation, an antihypertensive (e.g., an angiotensin-converting enzyme inhibitor) [for use in treating, e.g., HELLP syndrome], an anticoagulant, a corticosteroid (e.g., prednisone), or an immunosuppressive agent (e.g., vincristine or cyclosporine A). Examples of anticoagulants include, e.g., warfarin (Coumadin), aspirin, heparin, phenindione, fondaparinux, idraparinux, and thrombin inhibitors (e.g., argatroban, lepirudin, bivalirudin, or dabigatran). A composition described herein can also be formulated with a fibrinolytic agent (e.g., ancrod, ε-aminocaproic acid, antiplasmin-ai prostacyclin, and defibrotide) for the treatment of a complement-associated disorder. In some embodiments, a composition can be formulated with a lipid-lowering agent such as an inhibitor of hydroxymethylglutaryl CoA reductase. In some embodiments, a composition can be formulated with, or for use with, an anti-CD20 agent such as rituximab (RITUXAN™; Biogen Idec, Cambridge, Mass.). In some embodiments, e.g., for the treatment of RA, the composition can be formulated with one or both of infliximab (REMICADE®; Centocor, Inc.) and methotrexate (RHEUMATREX®, TREXALL®). In some embodiments, a composition described herein can be formulated with a non-steroidal anti-inflammatory drug (NSAID). Many different NSAIDS are available, some over the counter including ibuprofen (ADVIL@, MOTRIN®, NUPRIN®) and naproxen (ALLEVE®) and many others are available by prescription including meloxicam (MOBIC®), etodolac (LODINE®), nabumetone (RELAFEN®), sulindac (CLINORIL®), tolementin (TOLECTIN®), choline magnesium salicylate (TRILASATE®), diclofenac (CATAFLAM®, VOLTAREN®, ARTHROTEC®), Diflusinal (DOLOBID®), indomethicin (INDOCIN®Ketoprofen (ORUDIS®, ORUVAIL®), Oxaprozin (DAYPRO®), and piroxicam (FELDENE®). In some embodiments a composition can be formulated for use with an anti-hypertensive, an anti-seizure agent (e.g., magnesium sulfate), or an anti-thrombotic agent. Anti-hypertensives include, e.g., labetalol, hydralazine, nifedipine, calcium channel antagonists, nitroglycerin, or sodium nitroprussiate. (See, e.g., Mihu et al. (2007) J Gastrointestin Liver Dis 16(4):419-424.) Anti-thrombotic agents include, e.g., heparin, antithrombin, prostacyclin, or low dose aspirin.

In some embodiments, compositions including an engineered antibody as described herein can be formulated for administration with one or more additional therapeutic agents for use in treating a complement-associated disorder of the eye. Such additional therapeutic agents can be, e.g., bevacizumab or the Fab fragment of bevacizumab or ranibizumab, both sold by Roche Pharmaceuticals, Inc., and pegaptanib sodium (MUCOGEN®; Pfizer, Inc.). Such a kit can also, optionally, include instructions for administering the composition to a subject.

In some examples, the combination therapy can include administering to the subject one or more additional agents (e.g., an anti-IgE antibody, an anti-IL-4 antibody, an anti-IL-5 antibody, or an anti-histamine) that provide therapeutic benefit to a subject who has, is at risk of developing, or is suspected of having a complement-associated pulmonary disorder such as COPD or asthma.

In some embodiments, compositions formulated for intrapulmonary administration can include at least one additional active agent for treating a pulmonary disorder. The at least one active agent can be, e.g., an anti-IgE antibody (e.g., omalizumab), an anti-IL-4 antibody or an anti-IL-5 antibody, an anti-IgE inhibitor (e.g., montelukast sodium), a sympathomimetic (e.g., albuterol), an antibiotic (e.g., tobramycin), a deoxyribonuclease (e.g., PULMOZYME®), an anticholinergic drug (e.g., ipratropium bromide), a corticosteroid (e.g., dexamethasone), a β-adrenoreceptor agonist, a leukotriene inhibitor (e.g., zileuton), a 5-lipoxygenase inhibitor, a PDE inhibitor, a CD23 antagonist, an IL-13 antagonist, a cytokine release inhibitor, a histamine H1 receptor antagonist, an anti-histamine, an anti-inflammatory agent (e.g., cromolyn sodium), or a histamine release inhibitor.

In some embodiments, compositions can be formulated for administration to a subject along with intravenous gamma globulin therapy (IVIG), plasmapheresis, plasma replacement, or plasma exchange. In some embodiments, compositions can be formulated for use before, during, or after, a kidney transplant.

When compositions are to be used in combination with a second active agent, the compositions can be co-formulated with the second agent or the compositions can be formulated separately from the second agent formulation. For example, the respective pharmaceutical compositions can be mixed, e.g., just prior to administration, and administered together or can be administered separately, e.g., at the same or different times.

A composition described herein can replace or augment a previously or currently administered therapy. For example, upon treating with a composition described herein, administration of the one or more additional active agents can cease or diminish, e.g., be administered at lower levels, e.g., lower levels of a reference antibody comprising a heavy chain having the amino acid sequence of SEQ ID NO: 1 and a light chain having the amino acid sequence of SEQ ID NO: 2 following administration of an engineered antibody described herein. In some embodiments, administration of the previous therapy can be maintained. In some embodiments, a previous therapy will be maintained until the level of the composition reaches a level sufficient to provide a therapeutic effect. The two therapies can be administered in combination.

### Recombinant Gene Technology

In accordance with the present disclosure, there may be employed conventional molecular biology, microbiology, and recombinant DNA techniques within the skill of the art. Such techniques are described in the literature (see, e.g., Sambrook, Fritsch & Maniatis, Molecular Cloning: A Laboratory Manual, Second Edition (1989) Cold Spring Harbor Laboratory Press, Cold Spring Harbor, N.Y.; DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells and Enzymes (IRL Press, (1986)); B. Perbal, A Practical Guide To Molecular Cloning (1984); F. M. Ausubel et al. (eds.), Current Protocols in Molecular Biology, John Wiley & Sons, Inc. (1994).

Recombinant expression of a gene, such as a nucleic acid encoding a polypeptide, such as an engineered antibody described herein, can include construction of an expression vector containing a nucleic acid that encodes the polypeptide. Once a polynucleotide has been obtained, a vector for the production of the polypeptide can be produced by recombinant DNA technology using techniques known in the art. Known methods can be used to construct expression vectors containing polypeptide coding sequences and appropriate transcriptional and translational control signals. These methods include, for example, in vitro recombinant DNA techniques, synthetic techniques, and in vivo genetic recombination.

An expression vector can be transferred to a host cell by conventional techniques, and the transfected cells can then be cultured by conventional techniques to produce polypeptides.

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

### EXAMPLES

The following examples describe some of the preferred modes of making and practicing the present invention. However, it should be understood that these examples are for illustrative purposes only and are not meant to limit the scope of the invention.

### Example 1. In Vitro Assays for Evaluation of Potency of Anti-CFD mAb Inhibition

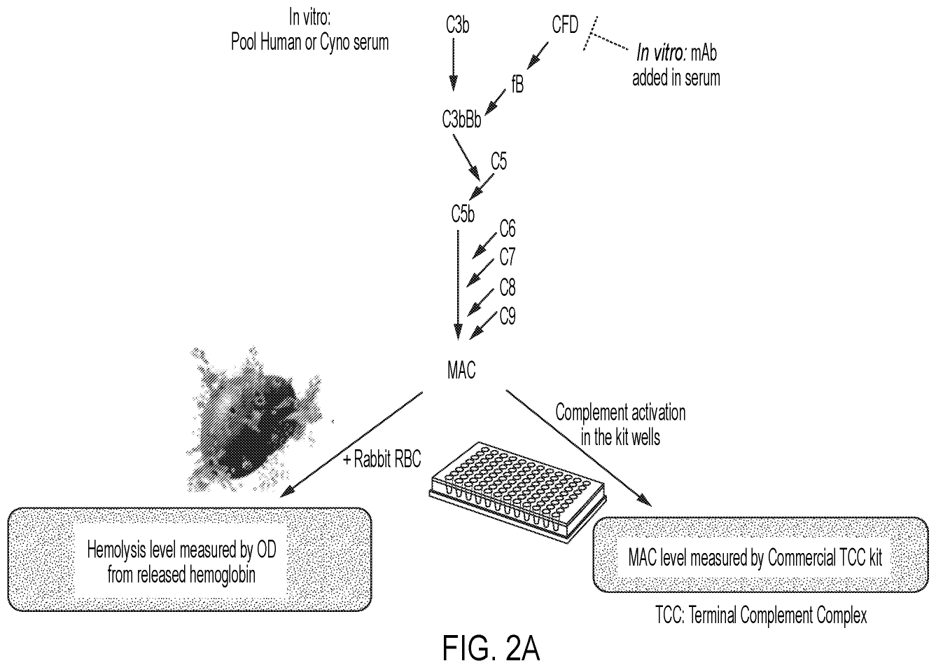

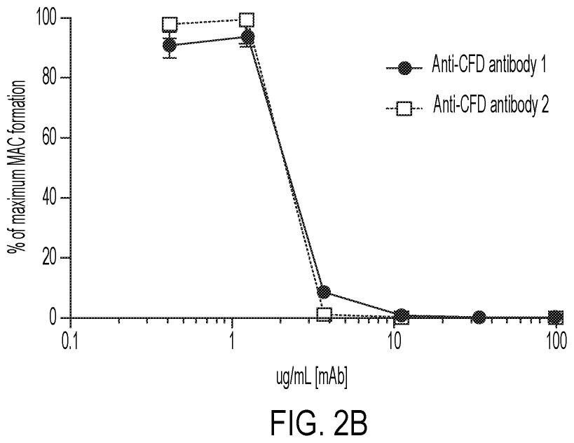

The present Example demonstrates in vitro assays for evaluation of potency of anti-CFD mAb inhibition. As shown in FIG. 2A, hemolysis levels can be measured by optical density (OD) from released hemoglobin and MAC levels can be measured using complement activation in a terminal complement complex (TCC) kit assay. Pooled human or cyno serum was combined with anti-CFD mAb 1 or anti-CFD mAb 2 (light chain SEQ ID NO: 1, heavy chain SEQ ID NO: 2). As shown in FIG. 2B, CFD depleted serum does not show any alternative pathway activity.

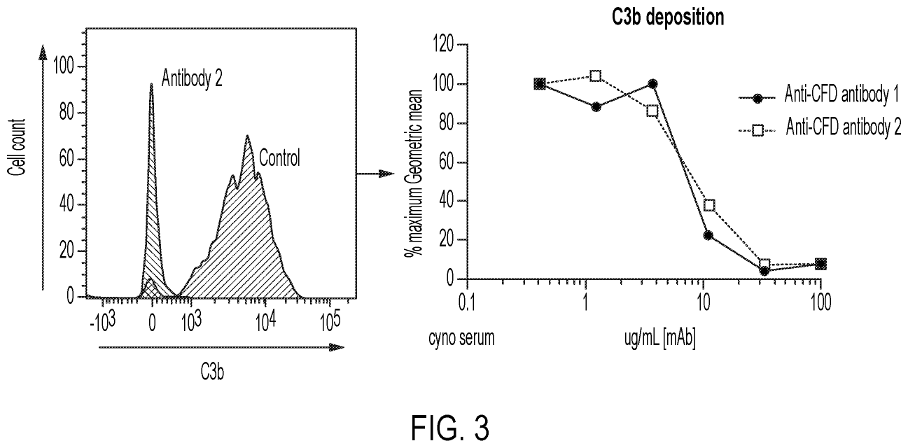

FIG. 3 shows percent maximum geometric mean of C3 deposition on RBCs using a hemolysis assay format. C5 depleted serum in contact to Rabbit RBC appeared to demonstrate C3b deposition on rabbit erythrocytes visualized by FACS. Anti-CFD antibody 2 inhibits C3b deposition on rabbit RBC similar to the benchmark antibody (anti-CFD antibody 1).

### Example 2. Acid Switching and Half-Life Extension of Anti-CFD Antibodies

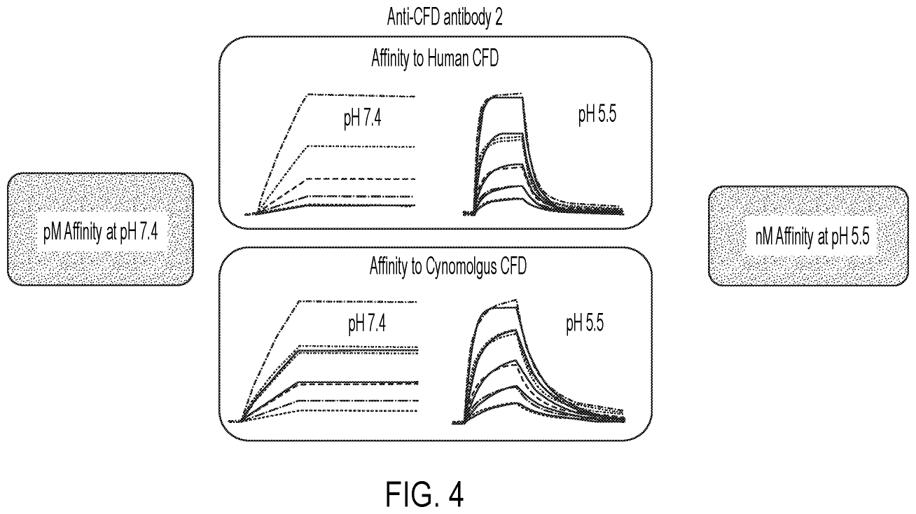

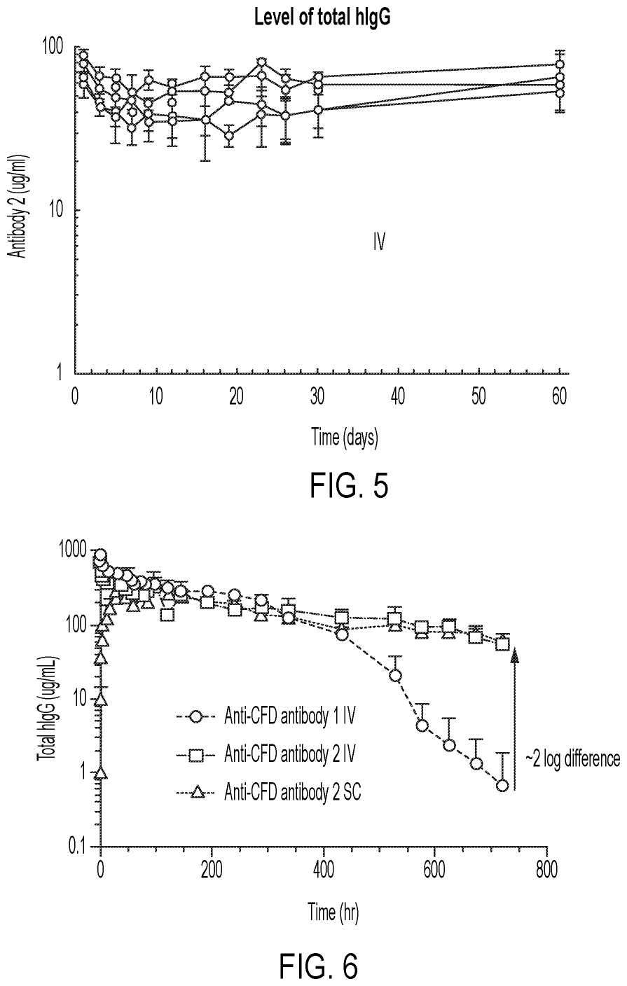

This example illustrates that the anti-CFD antibodies described herein demonstrate acid switch properties for human and cynomolgus CFD. As shown in FIG. 4 and Table 1, anti-CFD antibody 2 demonstrates binding affinity to human CFD and cyno CFD in the picomolar range at pH 7.4 and nM affinity at pH 5.5. Half-life experiments in in knock-in human FcRn mice showed that anti-CFD antibody 2 reaches a half-life greater than 45 days. (FIG. 5).

TABLE 1
Binding affinity of anti-CFD mAb at pH 5.5 and pH 7.4

| | KD cyno | | KD cyno | | KD human | | KD human | |
|---|---|---|---|---|---|---|---|---|
| | CFD | | CFD | | CFD | | CFD | |
| CFD | at pH | | at pH | | at pH | | at pH | |
| mAb | 7.4 | | 5.5 | | 7.4 | | 5.5 | |
| Antibody 2 | 37 | pM | 4.59 | nM | 2 | pM | 805 | nM |
| Antibody 1 | 31.8 | pM | 27.8 | pM | 4.78 | pM | 14.8 | pM |
| (benchmark) | | | | | | | | |

### Example 3. Pharmacokinetic and Pharmacodynamic (PK/PD) Evaluation of Anti-CFD mAbs in Non Human Primates (NHP)

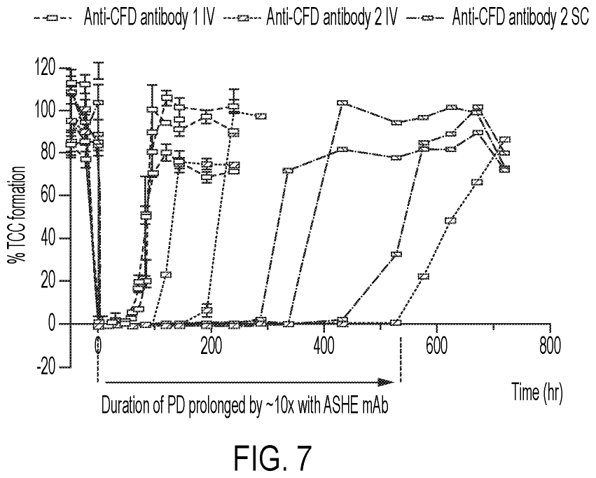

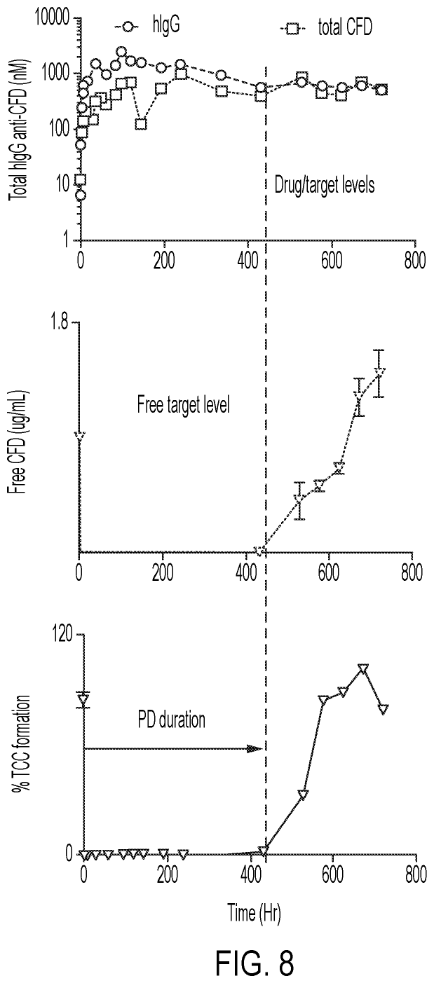

This example illustrates (PK/PD) evaluation of anti-CFD mAbs in Non Human Primates (NHP). In one study, animals were administered a single injection of 30 mg/kg of anti-CFD antibody 1 intravenously or antibody 2 intravenously or subcutaneously. Each treatment group included n=3 animals. Antibody 2 demonstrated an exteneded PK in cynomolgus monkeys compared to animals injected with antibody 1. As shown in FIG. 6, a 2 log difference in levels of total hIgG was observed in animals injected with anti-CFD mAb 2. ASHE properties demonstrated by anti-CFD antibody 2 translate to significantly improved PD duration. As shown in FIG. 7, the duration of PD was prolonged by approximately 10-fold with the ASHE mAb (antibody 2) compared to a control CFD antibody without ASHE properties. The duration of PD was linked to suppression of free target CFD (FIG. 8).

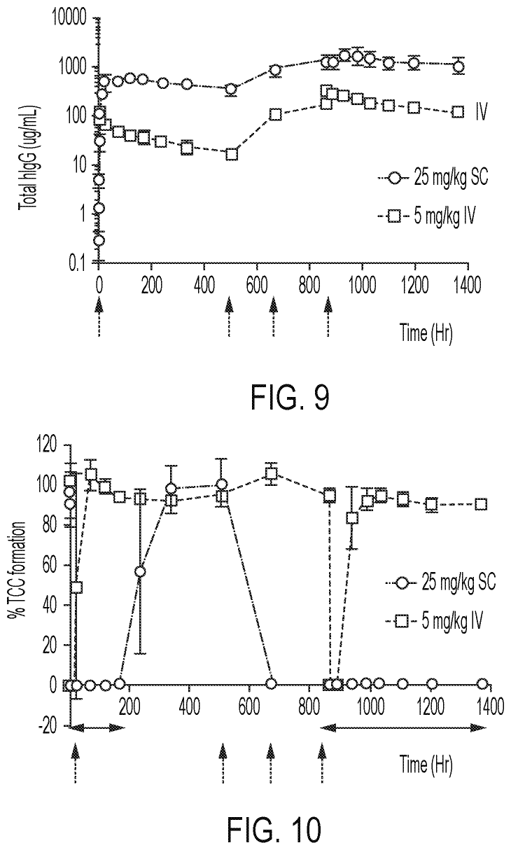

In a second study, animals were treated with multiple injections of anti-CFD antibody 2 at 50 mg/kg, 5 mg/kg, 1 mg/kg, intravenously or 25 mg/kg subcutaneously. Injections were administered on day 1 (time 0), day 22 (528 hrs), day 29 (696 hrs), and day 36 (864 hrs). Two animals were included in each does group. As shown in FIG. 9, an accumulation effect in total hIgG of the multiple injections on PK was observed after IV and SC injection. Improved PD duration was also observed in animals treated with multiple injections. Four subcutaneous injections of antibody 2 administered at 0 hr, 528 hr (22 days), 696 hr (29 days) and 864 hr (36 days) maintained complete inhibition for more than 504 hr (21 days) (FIG. 11). Subcutaneous injected anti-CFD ASHE mAb showed long PD duration of greater than 2 weeks. An IV administered anti-CFD antibody without ASHE properties (antibody 1) had a PD duration of 2 days.

### OTHER EMBODIMENTS

While a number of embodiments of this invention are described herein, the present disclosure and examples may be altered to provide other methods and compositions of this invention. Therefore, it will be appreciated that the scope of this invention is to be defined by the appended claims in addition to the specific embodiments that have been represented by way of example. All references cited herein are hereby incorporated by reference.

1. An isolated antibody that specifically binds to complement factor D (CFD) comprising
a heavy chain variable region amino acid sequence having at least 90%, 95%, 98%, or 99% sequence identity to SEQ ID NO: 5, 27, 29, 34 or 36; and/or
a light chain variable region amino acid sequence having at least 90%, 95%, 98%, or 99% sequence identity to SEQ ID NO: 6, 8, 26, 28, 35 or 37.

2. The isolated antibody of claim 2, comprising a heavy chain variable region amino acid sequence that is identical to SEQ ID NO: 5, 27, 29, 34 or 36.

3. The isolated antibody of any one of claims, comprising a light chain variable region amino acid sequence that is identical to SEQ ID NO: 6, 8, 26, 28, 35 or 37.

4. The isolated antibody of any one of claims 1-3, wherein the heavy chain CDR1 comprises the amino acid sequence YYAWS (SEQ ID NO: 12);
heavy chain CDR2 comprises the amino acid sequence DIANEGSTYYSPSLKS (SEQ ID NO: 15); and/or
heavy chain CDR3 comprises the amino acid sequence
(SEQ ID NO: 14)
LRSLYTDYDPHYYDY.

5. The isolated antibody of any one of claims 1-3, wherein the
heavy chain CDR1 comprises the amino acid sequence YYAWS (SEQ ID NO: 12);
heavy chain CDR2 comprises the amino acid sequence DIANEGSTYYSPSLES (SEQ ID NO: 20); and/or
heavy chain CDR3 comprises the amino acid sequence
(SEQ ID NO: 14)
LRSLYTDYDPHYYDY.

6. The isolated antibody of any one of claims 1-3, wherein the
heavy chain CDR1 comprises the amino acid sequence YYAWS (SEQ ID NO: 12);
heavy chain CDR2 comprises the amino acid sequence DIANDGSTYYSPSLES (SEQ ID NO: 13); and/or
heavy chain CDR3 comprises the amino acid sequence
(SEQ ID NO: 14)
LRSLYTDYDPHYYDY.

7. The isolated antibody of any one of claims 1-6, wherein the
light chain CDR1 comprises the amino acid sequence QGNLLPRHYAH (SEQ ID NO: 16);
light chain CDR2 comprises the amino acid sequence DDNIRPS (SEQ ID NO: 17); and/or
light chain CDR3 comprises the amino acid sequence QSASSNDDAV (SEQ ID NO: 18).

8. The isolated antibody of claim 7, wherein the
heavy chain CDR1 comprises the amino acid sequence YYAWS (SEQ ID NO: 12);
heavy chain CDR2 comprises the amino acid sequence
(SEQ ID NO: 15)
DIANEGSTYYSPSLKS;
heavy chain CDR3 comprises the amino acid sequence
(SEQ ID NO: 14)
LRSLYTDYDPHYYDY;
light chain CDR1 comprises the amino acid sequence QGNLLPRHYAH (SEQ ID NO: 16);
light chain CDR2 comprises the amino acid sequence DDNIRPS (SEQ ID NO: 17); and/or
light chain CDR3 comprises the amino acid sequence QSASSNDDAV (SEQ ID NO: 18).

9. The isolated antibody of claim 8, comprising:
a heavy chain variable region amino acid sequence that is identical to SEQ ID NO: 5; and
a light chain variable region amino acid sequence that is identical to SEQ ID NO: 6.

10. The isolated antibody of any one of claims 1-6, wherein the
light chain CDR1 comprises the amino acid sequence QGNLLPRHYAH (SEQ ID NO: 16);
light chain CDR2 comprises the amino acid sequence DDNIRPS (SEQ ID NO: 17); and/or
light chain CDR3 comprises the amino acid sequence QSADLNDDAV (SEQ ID NO: 19).

11. The isolated antibody of claim 10, wherein the
heavy chain CDR1 comprises the amino acid sequence YYAWS (SEQ ID NO: 12);
heavy chain CDR2 comprises the amino acid sequence
(SEQ ID NO: 15)
DIANEGSTYYSPSLKS;
heavy chain CDR3 comprises the amino acid sequence
(SEQ ID NO: 14)
LRSLYTDYDPHYYDY;
light chain CDR1 comprises the amino acid sequence QGNLLPRHYAH (SEQ ID NO: 16);
light chain CDR2 comprises the amino acid sequence DDNIRPS (SEQ ID NO: 17); and/or
light chain CDR3 comprises the amino acid sequence QSADLNDDAV (SEQ ID NO: 19).

12. The isolated antibody of claim 11, comprising:
a heavy chain variable region amino acid sequence that is identical to SEQ ID NO: 5; and
a light chain variable region amino acid sequence that is identical to SEQ ID NO: 8.

13. The isolated antibody of any one of claims 1-6, wherein the
light chain CDR1 comprises the amino acid sequence QGDLLPRHYAH (SEQ ID NO: 9);
light chain CDR2 comprises the amino acid sequence DDDIRPS (SEQ ID NO: 10); and/or
light chain CDR3 comprises the amino acid sequence QSADSNDDAV (SEQ ID NO: 11).

14. The isolated antibody of claim 13, wherein the
heavy chain CDR1 comprises the amino acid sequence YYAWS (SEQ ID NO: 12);
heavy chain CDR2 comprises the amino acid sequence
(SEQ ID NO: 20)
DIANEGSTYYSPSLES;
heavy chain CDR3 comprises the amino acid sequence LRSLYTDYDPHYYDY (SEQ ID NO: 14);
light chain CDR1 comprises the amino acid sequence QGDLLPRHYAH (SEQ ID NO: 9);
light chain CDR2 comprises the amino acid sequence DDDIRPS (SEQ ID NO: 10); and/or
light chain CDR3 comprises the amino acid sequence
(SEQ ID NO: 11)
QSADSNDDAV.

15. The isolated antibody of claim 14, comprising:
a heavy chain variable region amino acid sequence that is identical to SEQ ID NO: 29; and
a light chain variable region amino acid sequence that is identical to SEQ ID NO: 28.

16. The isolated antibody of claim 13, wherein the
heavy chain CDR1 comprises the amino acid sequence YYAWS (SEQ ID NO: 12);
heavy chain CDR2 comprises the amino acid seVWgDNA
(SEQ ID NO: 13)
DIANDGSTYYSPSLES;
heavy chain CDR3 comprises the amino acid sequence
(SEQ ID NO: 14)
LRSLYTDYDPHYYDY;
light chain CDR1 comprises the amino acid sequence QGDLLPRHYAH (SEQ ID NO: 9);
light chain CDR2 comprises the amino acid sequence DDDIRPS (SEQ ID NO: 10); and/or
light chain CDR3 comprises the amino acid sequence QSADSNDDAV (SEQ ID NO: 11).

17. The isolated antibody of claim 16, comprising:
a heavy chain variable region amino acid sequence that is identical to SEQ ID NO: 27; and
a light chain variable region amino acid sequence that is identical to SEQ ID NO: 26.

18. The isolated antibody of claim 6, wherein the
light chain CDR1 comprises the amino acid sequence QGNLLPRHYAH (SEQ ID NO: 16);
light chain CDR2 comprises the amino acid sequence DDNIRPS (SEQ ID NO: 17); and/or
light chain CDR3 comprises the amino acid sequence QSADSNDDAV (SEQ ID NO: 11).

19. The isolated antibody of claim 18, wherein
the heavy chain CDR1 comprises the amino acid sequence YYAWS (SEQ ID NO: 12);
the heavy chain CDR2 comprises the amino acid sequence
(SEQ ID NO: 13)
DIANDGSTYYSPSLES;
the heavy chain CDR3 comprises the amino acid sequence
(SEQ ID NO: 14)
LRSLYTDYDPHYYDY;
the light chain CDR1 comprises the amino acid sequence QGNLLPRHYAH (SEQ ID NO: 16);
the light chain CDR2 comprises the amino acid sequence DDNIRPS (SEQ ID NO: 17); and/or
the light chain CDR3 comprises the amino acid sequence QSADSNDDAV (SEQ ID NO: 11).

20. The isolated antibody of claim 19, comprising:
a heavy chain variable region amino acid sequence that is identical to SEQ ID NO: 34; and
a light chain variable region amino acid sequence that is identical to SEQ ID NO: 35.

21. The isolated antibody of claim 19, comprising:
a heavy chain variable region amino acid sequence that is identical to SEQ ID NO: 36; and
a light chain variable region amino acid sequence that is identical to SEQ ID NO: 37.

22. An isolated antibody that specifically binds complement factor D (CFD), wherein
the heavy chain CDR1 comprises the amino acid sequence YYAWS (SEQ ID NO: 12);
the heavy chain CDR2 comprises the amino acid sequence DIANDGSTYYSPSLES (SEQ ID NO: 13);
(SEQ ID NO: 13)
DIANDGSTYYSPSLES;
the heavy chain CDR3 comprises the amino acid sequence
(SEQ ID NO: 14)
LRSLYTDYDPHYYDY;
the light chain CDR1 comprises the amino acid sequence QGNLLPRHYAH (SEQ ID NO: 16);
the light chain CDR2 comprises the amino acid sequence DDNIRPS (SEQ ID NO: 17); and/or
the light chain CDR3 comprises the amino acid sequence QSADSNDDAV (SEQ ID NO: 11).

23. The isolated antibody of claim 22, comprising:
a heavy chain variable region amino acid sequence that is at least 90%, 91%, 92%, 93%, 94%, 95%, 96%, 97%, 98% or 99% identical to SEQ ID NO: 34; and
a light chain variable region amino acid sequence that is at least 90%, 91%, 92%, 93%, 94%, 95%, 96%, 97%, 98% or 99% identical to SEQ ID NO: 35.

24. The isolated antibody of claim 23, comprising:
a heavy chain variable region amino acid sequence that is identical to SEQ ID NO: 34; and
a light chain variable region amino acid sequence that is identical to SEQ ID NO: 35.

25. The isolated antibody of claim 22, comprising:
a heavy chain variable region amino acid sequence that is at least at least 90%, 91%, 92%, 93%, 94%, 95%, 96%, 97%, 98%, or 99% identical to SEQ ID NO: 36; and
a light chain variable region amino acid sequence that is at least 90%, 91%, 92%, 93%, 94%, 95%, 96%, 97%, 98% or 99% identical to SEQ ID NO: 37.

26. The isolated antibody of claim 25, comprising:
a heavy chain variable region amino acid sequence that is identical to SEQ ID NO: 36; and
a light chain variable region amino acid sequence that is identical to SEQ ID NO: 37.

27. The isolated antibody of any one of the preceding claims, wherein the antibody is a monoclonal antibody or fragment thereof.

28. The isolated antibody of any one of the preceding claims, further comprising an IgG constant region.

29. The isolated antibody of any one of the preceding claims comprising a light chain amino acid sequence having at least 85%, 90%, 95%, 98%, 99% or 100% sequence identity to SEQ ID NO: 1, 3, 30 or 32.

30. The isolated antibody of any one of the preceding claims comprising a heavy chain amino acid sequence having at least 85%, 90%, 95%, 98%, 99% or 100% sequence identity to SEQ ID NO: 2, 4, 31 or 33.

31. The isolated antibody of any one of the preceding claims, wherein the antibody inhibits the alternative complement pathway.

32. The isolated antibody of claim 31, wherein the antibody inhibits cleavage of complement factor B.

33. The isolated antibody of any one of the preceding claims, wherein the isolated antibody binds to CFD at pH 7.4 with an affinity dissociation constant (K_{D}) of between about 1 pM to about 50 pM.

34. The isolated antibody of any one of the preceding claims, wherein the isolated antibody binds to CFD at pH 7.4 with an affinity dissociation constant (K_{D}) of less than about 50 pM, less than about 45 pM, less than about 40 pM, less than about 35 pM, less than about 30 pM, less than about 25 pM, less than about 20 pM, less than about 15 pM, less than about 10 pM, less than about 9 pM, less than about 8 pM, less than about 7 pM, less than about 6 pM, or less than about 5 pM.

35. The isolated antibody of any one of the preceding claims, wherein the isolated antibody binds to CFD at pH 5.5 with an affinity dissociation constant (K_{D}) of between about 15 nM to about 150 nM.

36. The isolated antibody of any one of the preceding claims, wherein the isolated antibody binds to CFD at pH 5.5 with an affinity dissociation constant (K_{D}) of greater than about 15 nM, greater than about 20 nM, greater than about 25 nM, greater than about 30 nM, greater than about 35 nM, greater than about 40 nM, greater than about 45 nM, greater than about 50 nM, greater than about 100 nM, or greater than about 150 nM.

37. The isolated antibody of any one of the preceding claims, wherein the off rate of CFD from the antibody at pH 5.5 is greater than 0.010 s⁻¹, greater than 0.015 s⁻¹, greater than 0.02 s⁻¹, greater than 0.025 s⁻¹, greater than 0.03 s⁻¹, greater than 0.035 s⁻¹, or greater than 0.04 s⁻¹.

38. The isolated antibody of any one of the preceding claims, wherein the isolated antibody has a serum half-life of greater than about 5 days, about 10 days, about 15 days, about 20 days, about 25 days, about 30 days, about 35 days, about 40 days, about 45 days, about 50 days, about 60 days, about 70 days, about 80 days, about 90 days, about 95 days, about 100 days, about 125 days or longer.

39. A nucleic acid sequence encoding an isolated antibody of any one of claims 1-38.

40. A vector comprising the nucleic acid sequence of claim 39.

41. A host cell comprising the nucleic acid sequence of claim 39 or the vector of claim 40.

42. A method of producing an antibody, comprising culturing the host cell of claim 41 under conditions suitable for expression of the antibody.

43. An antibody, or antigen binding fragment thereof according to claims 1-38, for use as a medicament.

44. A method of treating a complement-mediated disease or disorder, the method comprising administering to a subject in need thereof an effective amount of the antibody of any one of claims 1-38.

45. The method of claim 44, wherein the complement mediated disease or disorder is atypical hemolytic uremic syndrome (aHUS) or Paroxysmal Nocturnal Hemoglobinuria (PNH).

46. The method of claim 44 or 45, wherein administration of the antibody inhibits intravascular hemolysis and extravascular hemolysis.
